(19) United States
(12) Patent Application Publication
(10) Pub. No.: US 2010/0137277 A1
(43) Pub. Date: Jun. 3, 2010

(54) PYRIDINE COMPOUNDS AND THEIR USE AS P2Y12 ANTAGONISTS

(75) Inventors: Thomas Antonsson, Molndal (SE); Peter Bach, Molndal (SE); David Brown, Molndal (SE); Ruth Bylund, Molndal (SE); Fabrizio Giordanetto, Molndal (SE); Johan Johansson, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/522,724

(22) PCT Filed: Jan. 11, 2008
(86) PCT No.: PCT/SE2008/000017

§ 371 Date: Jul. 10, 2009

Related U.S. Application Data
(60) Provisional application No. 60/884,658, filed on Jan. 12, 2007.

(51) Int. Cl. A61K 31/397; C07D 401/04; A61K 31/4545; A61P 7/02; A61K 31/4439

(52) U.S. Cl. 514/210.18; 546/194; 514/318; 546/268.1; 546/272.4; 514/340

(57) ABSTRACT

The present invention relates to certain new pyridin analogues of Formula (I) to processes for preparing such compounds, to their utility as P2Y₁₂ inhibitors and as anti-trombotic agents etc, their use as medicaments in cardiovascular diseases as well as pharmaceutical compositions containing them.

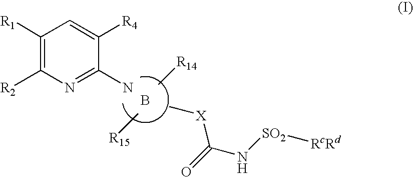

### FIELD OF THE INVENTION

The present invention provides novel pyridine compounds, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the anti-thrombotic effect of e.g. Aspirin (BMJ 1994; 308: 81-106 Antiplatelet Trialists' Collaboration. Collaborative overview of randomised trials of antiplatelet therapy, I: Prevention of death, myocardial infarction, and stroke by prolonged antiplatelet therapy in various categories of patients).

Platelet activation/aggregation can be induced by a variety of different agonists. However, distinct intracellular signalling pathways have to be activated to obtain full platelet aggregation, mediated via G-proteins G_{q}, G_{12/13} and Gᵢ (Platelets, A D Michelson ed., Elsevier Science 2002, ISBN 0-12-493951-1; 197-213: D Woulfe, et al. Signal transduction during the initiation, extension, and perpetuation of platelet plug formation) In platelets, the G-protein coupled receptor P2Y₁₂ (previously also known as the platelet P_{2T}, P2T_{ac}, or P2Y_{cyc} receptor) signals via Gi, resulting in a lowering of intra-cellular cAMP and full aggregation (Nature 2001; 409: 202-207 G Hollopeter, et al. Identification of the platelet ADP receptor targeted by antithrombotic drugs.). Released ADP from dense-granules will positively feedback on the P2Y12 receptor to allow full aggregation. WO 2002/098856 and WO 2004/052366 describe piperazino-carbonylmethylaminocarbonyl-naphtyl or -quinolyl derivatives as ADP receptor antagonist.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to is prevent Recurrent Events Trial Investigators. Effects of clopidogrel in addition to aspirin in patients with acute coronary syndromes without ST-segment elevation.). In these studies, the clinical benefit with a reduced bleeding risk as compared to thienopyridines (Sem Thromb Haemostas 2005; 31 (2): 195-204 J J J van Giezen & R G Humphries. Preclinical and clinical studies with selective reversible direct P2Y₁₂ antagonists. WO 2005/000281 describes a serie of pyrazolidine-3,5-dione derivatives and WO 2006/1147742 describes a serie of phenyl-pyrimidine derivatives which both series have been described as P2Y12 antagonists for the potential treatment of thrombosis. WO 2006/073361 discloses some P2Y12 antagonists for the potential treatment of thrombosis.

It is an object of the present invention to provide improved, potent, reversible and selective P2Y₁₂-antagonists as anti-trombotic agents.

### SUMMARY OF THE INVENTION

We have now surprisingly found that certain pyridine compounds of Formula (I) or a pharmaceutically acceptable salt thereof are reversible and selective P2Y₁₂ antagonists, hereinafter referred to as the compounds of the invention. The compounds of the invention unexpectedly exhibit beneficial properties that render them particularly suitable for use in the treatment of diseases/conditions as described below (See p. 77-78). Examples of such beneficial properties are high potency, high selectivity, and an advantageous therapeutic window.

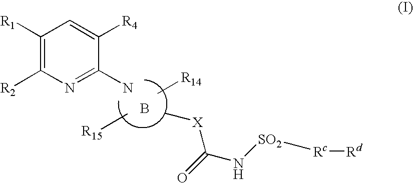

### DETAILED DESCRIPTION OF THE INVENTION

According to the present invention there is provided a novel compound of formula (I) or a pharmaceutically acceptable salt thereof:

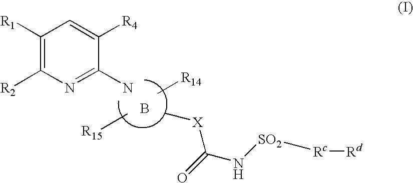

wherein
R₁ represents R₆OC(O), R₇C(O), R₁₆SC(O), R₁₇S, R₁₈C(S) or a group gII

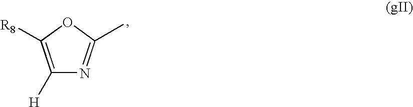

preferably R₁ represents R₆OC(O) or R₇C(O);

R₂ represents CN, halogen (F, Cl, Br, I), (C₄-C₈)alkyl optionally interrupted by oxygen and/or optionally substituted by OH, aryl, cycloalkyl, heterocyclyl; Furthermore R₂ represents (C₂-C₃)alkyl interrupted by oxygen; Furthermore R₂ represents (C₁-C₃)alkyl substituted by one or more of OH, awl, aryl(C₁-C₃)alkyloxy, cycloalkyl and heterocyclyl, with the proviso that any such OH group must be at least 2 carbon atoms away from any oxygen; further R₂ represents unsubstituted (C₁-C₁₂)alkoxy, (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, unsubstituted (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl;

R₄ represents H, CN, a halogen (F, Cl, Br, I) atom, (C₁-C₁₂)alkyl optionally interrupted by oxygen and/or optionally substituted by OH, COOH, (C₁-C₆)alkoxycarbonyl, or one or more halogen (F, Cl, Br, I) atoms; further R₄ represents hydroxy(C₁-C₁₂)alkyl, (C₁-C₁₂)alkoxy wherein the alkoxygroup may optionally be substituted by one or more halogen (F, Cl, Br, I) atoms, OH and/or COOH and/or (C₁-C₆)alkoxycarbonyl; further R₄ represents aryl(C₁-C₆)alkyl, (C₁-C₁₂)alkylsulfinyl, (C₁-C₁₂)alkylsulfonyl, (C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₃-C₆)cycloalkyl(C₁-C₁₂)alkoxy, aryl(C₁-C₆)alkoxy or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;

R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₁₂)alkyl, aryl or heterocyclyl;

R₇ represents (C₁-C₁₂)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₁₂)alkyl, aryl or heterocyclyl;

R₈ represents H, (C₁-C₁₂)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl;

R₁₄ represents H, OH with the proviso that the OH group must be at least 2 carbon atoms away from any heteroatom in the B ring/ring system, (C₁-C₈)alkyl optionally interrupted by oxygen and/or optionally substituted by one or more of OH, COOH and COOR^{e}; wherein R^{e} represents aryl, cycloalkyl, heterocyclyl or (C₁-C₈)alkyl optionally substituted by one or more of halogen (F, Cl, Br, I) atom(s), OH, aryl, cycloalkyl and heterocyclyl; further R₁₄ represents aryl, aryl(C₁-C₈)alkyl, aryl(C₁-C₃)alkoxy, heterocyclyl, a halogen (F, Cl, Br, I) atom, (C₃-C₆)cycloalkyl, (C₃-C₆)cycloalkyl(C₁-C₈)alkoxy, hydroxy(C₁-C₈)alkyl, (C₁-C₈)alkoxy, (C₃-C₆)cycloalkoxy, (C₁-C₈)alkylsulfinyl, (C₁-C₈)alkylsulfonyl, (C₁-C₈)alkylthio, (C₃-C₆)cycloalkylthio, or a group of formula NR^{a(14)}R^{b(14)} in which R^{a(14)} and R^{b(14)} independently represent H, (C₁-C₈)alkyl, (C₁-C₈)alkylC(O), (C₁-C₈)alkoxyC(O) or _{R}^{a(14)}and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atom(s), OH, aryl, cycloalkyl and heterocyclyl; further R₁₅ represents aryl, aryl(C₁-C₈)alkyl, aryl(C₁-C₃)alkoxy, heterocyclyl, a halogen (F, Cl, Br, I) atom, (C₃-C₆)cycloalkyl, (C₃-C₆)cycloalkyl(C₁-C₈)alkoxy, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, or a group of formula NR^{a(15)}R^{b(15)} in which R^{a(15)} and R^{b(15)} independently represent H, (C₁-C₁₂)alkyl, (C₁-C₁₂)alkylC(O)), (C₁-C₁₂)alkoxyC(O) or R^{a(15)} and R^{b(15)} together with the nitrogen atom represent piperidine, pyrrolidine, azetidine or aziridine;

R₁₆ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;

R₁₇ represents (C₁-C₁₂)alkyl optionally interrupted by oxygen and/or optionally to substituted by OH, aryl, cycloalkyl, heterocyclyl or one or more halogen (F, Cl, Br, I) atoms; further R₁₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R₁₈ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₁₂)alkyl,(C₁-C₁₂)alkoxy, (C₃-C₆)cycloalkoxy, aryl or heterocyclyl;

R^{e} is a direct bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₁₂)alkyl, (C₃-C₈)cycloalkyl, aryl or heterocyclyl, and anyone of s these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

Preferred values of each variable group or specific embodiments of variable groups or terms are as follows. Such values or embodiments may be used where appropriate with any of the values, definitions, claims, aspects, embodiments or embodiments of the invention defined hereinbefore or hereinafter. In particular, each may be used as an individual limitation on the broadest definition of formula (I).

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the particular definitions for that group.

It will be understood that when formula I compounds contain a chiral centre, the compounds of the invention may exist in, and be isolated in, optically active or racemic form. The invention includes any optically active or racemic form of a compound of formula I which act as P2Y₁₂ receptor antagonists. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a P2Y₁₂ receptor antagonist.

It will also be understood that in so far as compounds of the present invention exist as solvates, and in particular hydrates, these are included as part of the present invention.

It is also to be understood that generic terms such as “alkyl” include both the straight chain and branched chain groups such as butyl and tert-butyl. However, when a specific term such as “butyl” is used, it is specific for the straight chain or “normal” butyl group, branched chain isomers such as “t-butyl” being referred to specifically when intended.

In one embodiment alkyl is unsubstituted or substituted by one or more halogen (F, Cl, Br, I) atoms and/or one or more of the following groups, OH, CN, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the s nitrogen atom represent piperidine, pyrrolidine, azetidine or aziridine.

The term “alkyl” includes both linear or branched chain groups, unless otherwise specified, optionally substituted by one or more halogens (F, Cl, Br, I) or mixed halogen atoms.

One embodiment of alkyl when substituted by one or more halogen atoms (F, Cl, Br, I) is, for example, alkyl substituted by one or more fluorine atoms. Another embodiment of halogen substituted alkyl includes perfluoroalkyl groups such as trifluoromethyl.

The term “cycloalkyl” generally denotes a substituted or unsubstituted (C₃-C₆), unless other chain length specified, cyclic hydrocarbon.

In one embodiment cycloalkyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkoxy” includes both linear or branched chain groups, unless otherwise specified optionally substituted by one or more halogens (F, Cl, Br, I) or mixed halogen atoms.

The term aryl denotes a substituted or unsubstituted (C₆-C₁₄) aromatic hydrocarbon and includes, but is not limited to, phenyl, naphthyl, tetrahydronaphtyl, indenyl, indanyl, antracenyl, fenantrenyl, and fluorenyl.

In one embodiment aryl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic hetorocyclic groups, and includes, but is not limited to azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine as well as pyridine-N-oxide, piperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, eg. R₄ when selected as heterocyclyl may be a furan, when R^{d} (also when selected as heterocyclyl) may be a pyrrole.

In one embodiment heterocyclyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alicyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloallcyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

In another embodiment of the invention the heterocyclyl group comprises an aromatic 5-membered or 6-membered heterocyclic ring containing one, two or three heteroatoms selected from nitrogen, oxygen and sulphur, and an aromatic 5-membered or 6-membered heterocyclic ring containing one, two or three heteroatoms selected from nitrogen, oxygen and sulphur which is fused to a benzene ring;

In an alternative embodiment of the invention the heterocyclyl group is a non-aromatic 5-membered or 6-membered heterocyclic ring containing one, two or three heteroatoms selected from nitrogen, oxygen and sulphur, fused to a benzene ring.

In a further embodiment of the invention the heterocyclyl group is a group chosen among furyl, pyrrolyl, thienyl, pyridyl, N-oxido-pyridyl, pyrazinyl, pyrimidinyl, pyridazinyl, imidazolyl, oxazolyl, isooxazolyl, thiazolyl, isothiazolyl, oxadiazolyl, 1,2,3-triazolyl, 1,2,4-triazolyl, benzfuranyl, quinolyl, isoquinolyl, benzimidazolyl, indolyl, benzdihydrofuranyl, benzodioxolyl (such as 1,3-benzodioxolyl), benzoxadiazole, dihydrobenzodioxin, benzothiophene, benzothiadiazole, imidazothiazole, 2,3-dihydrobenzofuran, isoxazole, dihydropyrazole and benzdioxanyl (such as 1,4-benzdioxanyl). More particular values include, for example, furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole, dihydropyrazole and benzdioxanyl (such as 1,4-benzdioxanyl).

In an even further embodiment of the invention the heterocyclyl group is a group chosen among furyl, pyrrolyl, thienyl, pyridyl, N-oxido-pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole or dihydropyrazole.

In one embodiment of the invention R₁ represents R₆OC(O).

In another embodiment of the invention R₁ represents R₁₆SC(O).

In yet another embodiment of the invention R₁ represents R₇C(O).

In still another embodiment of the invention R₁ represents R₆OC(O) or R₇C(O).

In yet a further embodiment R₁ represents a group (gII),

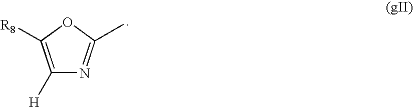

In a further embodiment of the invention R₁ is selected among R₆OC(O) and R₁₆SC(O) wherein R₆ can be methyl, ethyl, 2,2,2-trifluoroethyl, isopropyl, cyclo-propyl, iso-butyl, n-butyl, cyclo-butyl, n-propyl, tertbutyl, cyclo-pentyl, 2,2-dimethylpropyl, benzyl and 4-fluorobenzyl and wherein R₁₆ is ethyl.

In yet a even further embodiment of the invention R₁ is selected among R₆OC(O) and R₇C(O) wherein R₆ can be methyl, ethyl, 2,2,2-trifluoroethyl, isopropyl, cyclo-propyl, iso-butyl, n-butyl, cyclo-butyl, n-propyl, tertbutyl, cyclo-pentyl, 2,2-dimethylpropyl, benzyl and 4-fluorobenzyl and wherein R₇ is selected among (C₁-C₆)alkyl.

In another further embodiment of the invention R₁ is selected among R₆OC(O) and R₇C(O) wherein R₆ can be ethyl and isopropyl, and wherein R₇ is selected among propyl and butyl.

R₁ may also be embodified by the group gII,

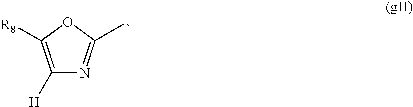

in which R₈ is selected from H, (C₁-C₆)alkyl, such as methyl or ethyl.

In another embodiment for the group R₈ this group can be chosen among hydrogen, methyl, ethyl, n-propyl and n-butyl.

Embodiments for R₂ include, for example (C₁-C₃)alkyl substituted by one or more of OH, aryl, aryl(C₁-C₃)alkyloxy, cycloalkyl and heterocyclyl, with the proviso that any such OH group must be at least 2 carbon atoms away from any oxygen.

In one embodiment of the invention R₂ is represented by unsubstituted (C₁-C₃)alkyloxy or unsubstituted (C₁-C₃)alkylthio.

Other embodiments for R₂ are phenyl, methoxy and ethoxy.

In another embodiment R₂ is selected from the group consisting of CN, unsubstituted alkoxy and unsubstituted alkylthio.

In a further embodiment R₂ is selected from the group consisting of CN, methoxy, ethoxy, methylthio and ethylthio.

Embodiments for R₄ include H, halogen such as chloro, methyl, cyano, nitro, amino unsubstituted or optionally substituted with one or two methyl groups and further includes 4-methoxy-4-oxobutoxy, 3-carboxy-propoxy and methylcarbonyl.

In a further embodiment R₄ is selected from the group consisting of CN and halogen.

In an even further embodiment R₄ is selected from the group consisting of CN and chloro (Cl).

In one embodiment of the invention R₇ is (C₁-C₆)alkyl.

In a further embodiment R₇ is chosen among propyl and butyl.

Further embodiments for R₈ include, hydrogen, methyl and ethyl.

Further embodiments for R₁₄ include, for example, hydrogen, methyl, amino, tert-butyloxycarbonyl, tert-butyloxycarbonyl-imino, 2-carboxyethyl and 3-tert-butoxy-3-oxo-propyl.

Other further embodiments for R₁₄ include, for example, hydrogen, methyl, tert-butyloxycarbonyl-imino, and amino.

In one embodiment of the invention R₁₅ represents H.

In one embodiment of the invention R^{d} represents (C₁-C₁₂)alkyl.

Further embodiments for R^{d} includes aryl or heterocyclyl, more particularly, aryl or aromatic heterocyclyl.

Another embodiment for R^{d} include, aryl such as phenyl and aromatic heterocyclyl such as thienyl.

Other embodiments of R^{d} include phenyl which optionally may be substituted.

In a special embodiment R^{d} represents aryl, heterocyclyl or (C₃-C₆)cycloalkyl, and anyone of these groups are optionally substituted with one or more halogen (F, Cl, Br, I) atoms or mixed halogen atoms, and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine.

Even further embodiments for R^{d} include phenyl optionally substituted at the 2,3,4 or 5-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2,3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl,benzothien-3-yl, 2,5-dimethyl-3-thienyl, 3-thienyl, 2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenypsulfonyl]-3-methyl-2-thienyl, 5-(methoxycarbonyl)-2-furyl and 4-(methoxycarbonyl)-5-methyl-2-furyl.

Even another further embodiments for R^{d} include phenyl optionally substituted at the 2,3,4,5 or 6-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2,3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl,benzothien-3-yl, 2,5-dimethyl-3-thienyl, 3-thienyl, 2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

In one embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c} R^{d} represents an aryl-(C₁-C₄)alkylene group with any substituents according to above.

In a preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-(C₁-C₃)alkylene group with any substituents according to above.

In a further embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i e. R^{c}R^{d} represents a heterocyclyl-(C₁-C₄)alkylene group with any substituents according to above.

In a further preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i e. R^{c}R^{d} represents a heterocyclyl-(C₁-C₃)alkylene group with any substituents according to above.

In a particular embodiment of the invention R^{c} represents a C₁-alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-C₁-alkylene group with any substituents according to above.

In a further particular embodiment of the invention R^{c} represents an unsubstituted s or monosubstituted or disubstituted C₁-alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-C₁-alkylene group with any substituents according to above.

In one embodiment of the invention R₁₉ represents hydrogen.

In another embodiment of the invention R₁₉ represents methyl.

In a most particular embodiment of the invention R^{c}R^{d} represents a benzyl group, or a benzyl group which is substituted according to what is described in connection to substitution of the aryl group.

In one embodiment of the invention X represents a single bond.

In another embodiment of the invention X represents imino (—NH—) or methylene (—CH₂—). In yet another embodiment X represents imino (—NH—). In a further embodiment X represents methylene (—CH₂—).

Suitable values for the B ring/ring system include, for example, diazepanylene, piperazinylene, piperidinylene, pyrrolidinylene and azetidinylene, wherein anyone of them may be presents in any of their isomeric forms (e.g. piperazin-tetrahydropyridazin-tetrahydropyrimidin).

Embodiments for the B ring/ring system include, for example, diazepanylene, piperazinylene, piperidinylene, pyrrolidinylene and azetidinylene. Further embodiments include these groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₁₂)alkyl optionally substituted by one or more of halogen (F, Cl, Br, I) or mixed halogen s atoms, OH, aryl, cycloalkyl and heterocyclyl.

In an alternative to the embodiment for the B ring/ring system above, the embodiment include, for example, diazepanylene, piperazinylene, piperidinylene, pyrrolidinylene or azetidinylene groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₆)alkyl optionally substituted by one or more of halogen (F, Cl, Br, I) or mixed halogen atoms, OH, aryl, cycloalkyl and heterocyclyl.

A 2nd embodiment of formula I is defined by;
R₁ represents R₆OC(O), R₇C(O), R₁₆SC(O), R₁₇S, R₁₈C(S) or a group gII

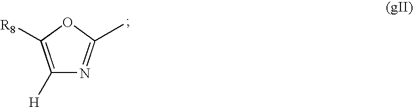

R₂ represents CN, halogen (F, Cl, Br, I), (C₄-C₆)alkyl optionally interrupted by oxygen and/or optionally substituted by OH, aryl, cycloalkyl, heterocyclyl; Furthermore R₂ represents (C₂-C₃)alkyl interrupted by oxygen; Furthermore R₂ represents (C₁-C₃)alkyl substituted by one or more of OH, aryl, aryl(C₁-C₃)alkyloxy, cycloalkyl and heterocyclyl, with the proviso that any such OH group must be at least 2 carbon atoms away from any oxygen; further R₂ represents unsubstituted (C₁-C₆)alkoxy, (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, unsubstituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl;

R₄ represents H, CN, a halogen (F, Cl, Br, I) atom, (C₁-C₆)alkyl optionally interrupted by oxygen and/or optionally substituted by OH, COOH, (C₁-C₆)alkoxycarbonyl, or one or more halogen (F, Cl, Br, I) atoms; further R₄ represents hydroxy(C₁-C₆)alkyl, (C₁-C₆)alkoxy wherein the alkoxygroup may optionally be substituted by one or more halogen (F, Cl, Br, I) atoms, OH and/or COOH and/or (C₁-C₆)alkoxycarbonyl; further R₄ represents aryl(C₁-C₆)alkyl, (C₁-C₆)alkylsulfinyl, (C₁-C₆)alkylsulfonyl, (C₁-C₆)alkylthio, (C₃-C₆)cycloalkyl(Cₗ-C₆)alkylsulfinyl, (C₃-C₆)cycloalkyl(C₁-C₆)alkylsulfonyl, (C₃-C₆)cycloalkyl(C₁-C₆)alkoxy, aryl(C₁-C₆)alkoxy or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl, heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atom(s), OH, aryl, cycloalkyl and heterocyclyl; further R₁₄ represents aryl, aryl(C₁-C₆)alkyl, aryl(C₁-C₃)alkoxy, heterocyclyl, a halogen (F, Cl, Br, I) atom, (C₃-C₆)cycloalkyl, (C₃-C₆)cycloalkyl(C₁-C₆)alkoxy, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom to represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atom(s), OH, aryl, cycloalkyl and heterocyclyl; further R₁₅ represents aryl, aryl(C₁-C₆)alkyl, aryl(C₁-C₃)alkoxy, heterocyclyl, a halogen (F, Cl, Br, I) atom, (C₃-C₆)cycloalkyl, (C₃-C₆)cycloalkyl(C₁-C₆)alkoxy, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, or a group of formula NR^{a(15)}R^{b(15)} in which R^{a(15)} and R^{b(15)} independently represent H, (C₁-C₆)alkyl, (C₁-C₆)alkylC(O)), (C₁-C₆)alkoxyC(O) or R^{a(15)} and R^{b(15)} together with the nitrogen atom represent piperidine, pyrrolidine, azetidine or aziridine;

R₁₆ represents (C₁-C₆)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₆)alkyl, (C₁-C₆)alkoxy, (C₃-C₆)cycloalkoxy, aryl or heterocyclyl;

R¹⁷ represents (C₁-C₆)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₆)alkyl, (C₁-C₆)alkoxy, (C₃-C₆)cycloalkoxy, aryl or heterocyclyl;

R₁₈ represents (C₁-C₆)alkyl optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₆)alkyl, (C₁-C₆)alkoxy, (C₃-C₆)cycloalkoxy, aryl or heterocyclyl;

R^{c} is a direct bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} is individually and independently from each other represents hydrogen, (C₁-C₄)alkyl or R^{a(Rc)} and R^{b(Rc)} together with the nitrogen atom represent piperidine, pyrrolidine, azetidine or aziridine; Further Re represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₆)alkyl, (C₃-C₆)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, s pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 3rd embodiment of formula I is defined by;
R₁ represents R₆OC(O), R₁₆SC(O) or a group gII

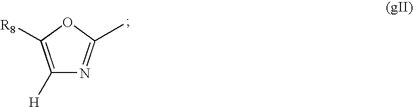

R₂ represents CN, halogen (F, Cl, Br, I), (C₄-C₆)alkyl optionally interrupted by oxygen and/or optionally substituted by OH, aryl, cycloalkyl, heterocyclyl; Furthermore R₂ represents (C₂-C₃)alkyl interrupted by oxygen; Furthermore R₂ represents (C₁-C₃)alkyl substituted by one or more of OH, aryl, aryl(C₁-C₃)alkyloxy, cycloalkyl and heterocyclyl, with the proviso that any such OH group must be at least 2 carbon atoms away from any oxygen; further R₂ represents unsubstituted (C₁-C₆)alkoxy, (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, unsubstituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl;

R₄ represents H, CN, a halogen (F, Cl, Br, I) atom, (C₁-C₆)alkyl optionally interrupted by oxygen and/or optionally substituted by OH, COOH, (C₁-C₆)alkoxycarbonyl, or one or more halogen (F, Cl, Br, I) atoms; further R₄ represents hydroxy(C₁-C₆)alkyl, (C₁-C₆)alkoxy wherein the alkoxygroup may optionally be substituted by one or more halogen (F, Cl, Br, I) atoms, OH and/or COOH and/or (C₁-C₆)alkoxycarbonyl; further R₄ represents aryl(C₁-C₆)alkyl, (C₁-C₆)alkylthio, or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl, heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atom(s), OH, aryl, cycloalkyl and heterocyclyl; further R₁₄ represents aryl, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atom(s), OH, aryl, cycloalkyl and heterocyclyl; further R₁₄ represents aryl, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O)), (C₁-C₆)alkoxyC(O) or R^{a(15)} and R^{b(15)} together with the nitrogen atom represent piperidine, pyrrolidine, azetidine or aziridine;

R₁₆ represents (C₁-C₆)alkyl optionally interrupted by oxygen and/or optionally substituted by OH, aryl, cycloalkyl, heterocyclyl or one or more halogen (F, Cl, Br, I) atoms;

R^{c} is a direct bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, ox_{Y}-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₆)alkyl, (C₃-C₆)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

An alternative 3rd embodiment of formula I is defined by;
R₁ represents R₆OC(O), R₇C(O) or a group gII

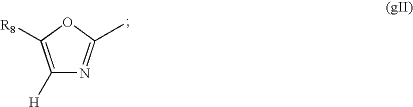

R₂ represents CN, halogen (F, Cl, Br, I), (C₄-C₆)alkyl optionally interrupted by oxygen and/or optionally substituted by OH, aryl, cycloalkyl, heterocyclyl; Furthermore R₂ represents (C₂-C₃)alkyl interrupted by oxygen; Furthermore R₂ represents (C₁-C₃)alkyl substituted by one or more of OH, aryl, aryl(C₁-C₃)alkyloxy, cycloalkyl and heterocyclyl, with the proviso that any such OH group must be at least 2 carbon atoms away from any oxygen; further R₂ represents unsubstituted (C₁-C₆)alkoxy, (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, unsubstituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl;

R₄ represents H, CN, a halogen (F, Cl, Br, I) atom, (C₁-C₆)alkyl optionally interrupted by oxygen and/or optionally substituted by OH, COOH, (C₁-C₆)alkoxycarbonyl, or one or more halogen (F, Cl, Br, I) atoms; further R₄ represents hydroxy(C₁-C₆)alkyl, (C₁-C₆)alkoxy wherein the alkoxygroup may optionally be substituted by one or more halogen (F, Cl, Br, I) atoms, OH and/or COOH and/or (C₁-C₆)alkoxycarbonyl; further R₄ represents aryl(C₁-C₆)alkyl, (C₁-C₆)alkylthio, or a group of formula N^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl, heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atom(s), OH, aryl, cycloalkyl and heterocyclyl; further R₁₄ represents aryl, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally s substituted by one or more of halogen (F, Cl, Br, I) atom(s), OH, aryl, cycloalkyl and heterocyclyl; further R₁₄ represents aryl, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O)), (C₁-C₆)alkoxyC(O) or R^{a(15)} and R^{b(15)} together with the nitrogen atom represent piperidine, pyrrolidine, azetidine or aziridine;

R^{c} is a direct bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further Re represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₆)alkyl, (C₃-C₆)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 4rth embodiment of formula I is defined by;
R₁ represents R₆OC(O), R₁₆SC(O) or a group gII

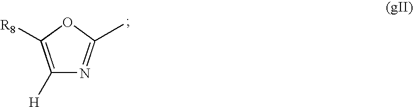

R₂ represents CN, halogen (F, Cl, Br, I), (C₄-C₆)alkyl optionally interrupted by oxygen and/or optionally substituted by OH, aryl, cycloalkyl, heterocyclyl; Furthermore R₂ represents (C₂-C₃)alkyl interrupted by oxygen; Furthermore R₂ represents (C₁-C₃)alkyl substituted by one or more of OH, aryl, aryl(C₁-C₃)alkyloxy, cycloalkyl and heterocyclyl, with the proviso that any such OH group must be at least 2 carbon atoms away from any oxygen; further R₂ represents unsubstituted (C₁-C₆)alkoxy, hydroxy(Cₗ-C₆)alkyl, (C₃-C₆)cycloalkoxy, unsubstituted (C₁-C₆)alkylthio, (C₃-C₆)cycloalkylthio, arylthio, aryl(C₁-C₆)alkylthio, heterocyclyl(C₁-C₆)alkylthio, (C₃-C₆)cycloalkyl(C₁-C₆)alkylthio;

R₄ represents CN, a halogen (F, Cl, Br, I) atom; further R₄ represents hydroxy(C₁-C₆)alkyl, (C₁-C₆)alkoxy wherein the alkoxygroup may optionally be substituted by one or more halogen (F, Cl, Br, I) atom(s), OH and/or COOH and/or (C₁-C₆)alkoxycarbonyl;

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl or hydroxy(C₂-C₆)alkyl;

R₈ represents H, (C₁-C₆)alkyl optionally interrupted by oxygen, and/or optionally substituted by aryl, cycloalkyl, heterocyclyl or one or more halogen (F, Cl, Br, I) atoms;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atom(s), OH, aryl, cycloalkyl and heterocyclyl; further R₁₄ represents a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H;

R₁₆ represents (C₁-C₄)alkyl;

R^{c} is a direct bond or represents an unsubstituted or monosubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl; Further R^{c} represents imino (—NH—) or N-substituted imino (—NR₁₉—);

R₁₉ represents H or methyl;

R^{d} represents (C₁-C₆)alkyl, (C₃-C₆)cycloalkyl, aryl or heterocyclyl, and anyone of these groups optionally substituted with one or more halogen (F, Cl, Br, I) atoms and/or one or more of the following groups, CN, NO₂, (C₁-C₆)alkyl, (C₁-C₆)alkoxy, halogen substituted (C₁-C₆)alkyl;

X represents a single bond, imino (—NH—) or methylene (—CH₂—); and

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

An alternative 4rth embodiment of formula I is defined by;
R₁ represents R₆OC(O), R₇C(O) or a group gII

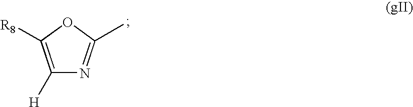

R₂ represents CN, halogen (F, Cl, Br, I), (C₄-C₆)alkyl optionally interrupted by oxygen and/or optionally substituted by OH, aryl, cycloalkyl, heterocyclyl; Furthermore R₂ represents (C₂-C₃)alkyl interrupted by oxygen; Furthermore R₂ represents (C₁-C₃)alkyl substituted by one or more of OH, aryl, aryl(C₁-C₃)alkyloxy, cycloalkyl and heterocyclyl, with the proviso that any such OH group must be at least 2 carbon atoms away from any oxygen; further R₂ represents unsubstituted (C₁-C₆)alkoxy, hydroxy(C₁-C₆)alkyl, (C₃-C₆)cycloalkoxy, unsubstituted (C₁-C₆)alkylthio, (C₃-C₆)cycloalkylthio, arylthio, aryl(C₁-C₆)alkylthio, heterocyclyl(C₁-C₆)alkylthio, (C₃-C₆)cycloalkyl(C₁-C₆)alkylthio;

R₄ represents CN, a halogen (F, Cl, Br, I) atom; further R₄ represents hydroxy(C₁-C₆)alkyl, (C₁-C₆)alkoxy wherein the alkoxygroup may optionally be substituted by one or more halogen (F, Cl, Br, I) atom(s), OH and/or COOH and/or (C₁-C₆)alkoxycarbonyl;

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl or hydroxy(C₂-C₆)alkyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms;

R₈ represents H, (C₁-C₆)alkyl optionally interrupted by oxygen, and/or optionally substituted by aryl, cycloalkyl, heterocyclyl or one or more halogen (F, Cl, Br, I) atoms;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atom(s), OH, aryl, cycloalkyl and heterocyclyl; further R₁₄ represents a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H;

R^{c} is a direct bond or represents an unsubstituted or monosubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl; Further R^{e} represents imino (—NH—) or N-substituted imino (—NR₁₉—);

R₁₉ represents H or methyl;

R^{d} represents (C₁-C₆)alkyl, (C₃-C₆)cycloalkyl, awl or heterocyclyl, and anyone of these groups optionally substituted with one or more halogen (F, Cl, Br, I) atoms and/or one or more of the following groups, CN, NO₂, (C₁-C₆)alkyl, (C₁-C₆)alkoxy, halogen substituted (C₁-C₆)alkyl;

X represents a single bond, imino (—NH—) or methylene (—CH₂—); and

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 5th embodiment of formula I is defined by that;
R₁ is chosen from the group consisting of ethoxycarbonyl, ispropyloxycarbonyl, n-propylcarbonyl and n-butylcarbonyl;
R₂ is chosen from the group consisting of methoxy, ethoxy, methylthio, ethylthio, cyano, chloro, hydroxymethyl, ethoxymethyl, 2-methoxyethyl, (benzoyloxy)methyl, ((3,4-dimethoxybenzyl)oxy)methyl, 1H-1,2,4-triazol-1-yl-methyl, 1H-1,2,3-triazol-1-yl-methyl, and 1H-imidazol-1-yl-methyl;
R₃ is H;
R₄ is chosen from the group consisting of CN, chloro and fluoro;
R₆ is ethyl or isopropyl;
R₇ is n-propyl or n-butyl;
R₁₄ is H;
R₁₅ is H;
R^{c} is a single bond or methylene (—CH₂—);
R^{d} is chosen from the group consisting of phenyl, 2-fluorophenyl, 3-fluorophenyl, 4-fluorophenyl, 2-chlorophenyl, 4-chlorophenyl, 4-(trifluoromethyl)phenyl, 3,4-difluorophenyl, 2,4-difluorophenyl, 2,3-difluorophenyl, 2,4-dichlorophenyl, 2-chloro-4-fluorophenyl, 4-methoxy-phenyl and 4-chloro-2-fluorophenyl;
X is a single bond; and
B is chosen from the group consisting of 3-azetidin-1-ylene and 4-piperidin-1-ylene, and the substituents R₁₄ and R₁₅ are connected to the B ring/ring system, in such a way that no quarternary ammonium compounds are formed (by these connections).

In a 6th embodiment of formula (I), formula (I) is defined as being any compound(s) of formula (Ia)-(Ii):

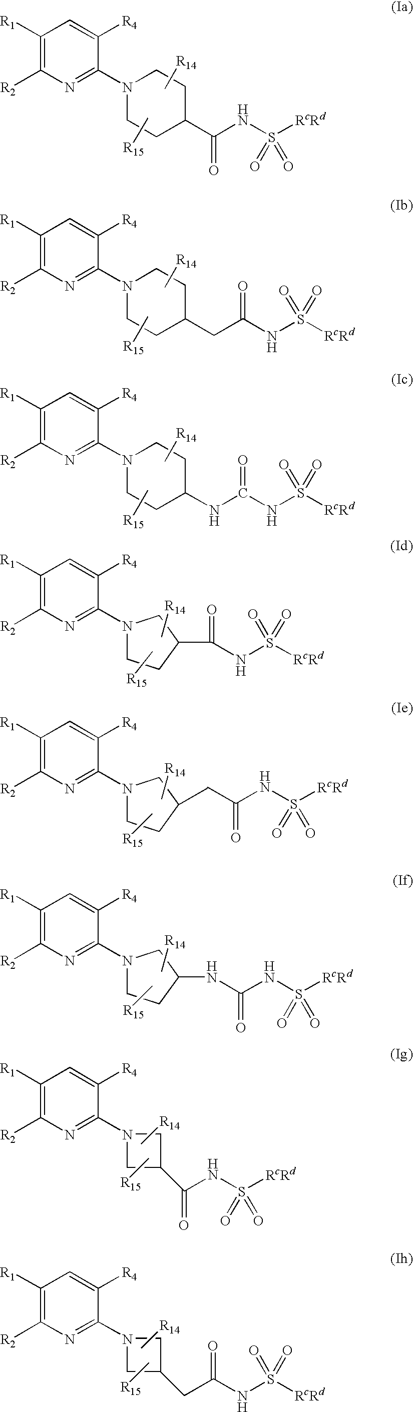

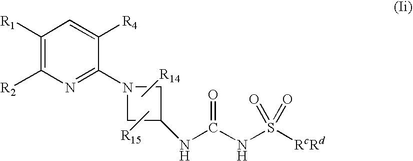

In the above Ia to Ii the various values of R are as defined above and include any of the previously mentioned embodiments.
In a 7^{th} embodiment formula (I) is defined as being any compound(s) of formula (Iaa)-(Ijj);

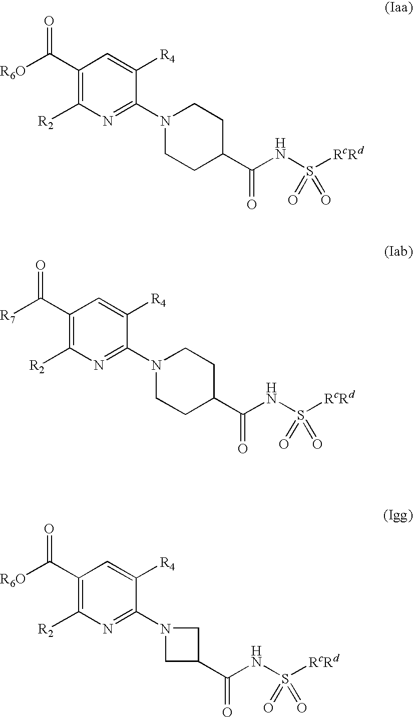

In the above Iaa to Igg the various values of R (except R₅, R₁₄ and R₁₅, all being H) are as defined above and include any of the previously mentioned embodiments.

Examples of specific compounds according to the invention can be selected from;
ethyl 6-{4-[(benzylsulfonyl)carbamoyl]piperidin-1-yl}-5-cyano-2-methoxynicotinate
ethyl 6-{3-[(benzylsulfonyl)carbamoyl]azetidin-1-yl}-5-cyano-2-methoxynicotinate
ethyl 6-{4-[(benzylsulfonyl)carbamoyl]piperidin-1-yl}-5-cyano-2-ethoxynicotinate
ethyl 6-{4-[(benzylsulfonyl)carbamoyl]piperidin-1-yl}-5-cyano-2-(ethylthio)nicotinate
ethyl 6-{4-[(benzylsulfonyl)carbamoyl]piperidin-1-yl}-2,5-dicyanonicotinate
ethyl 6-{4-[(benzylsulfonyl)carbamoyl]piperidin-1-yl}-5-cyano-2-(hydroxymethypnicotinate
ethyl 5-cyano-2-methoxy-6-{4-[(phenylsulfonyl)carbamoyl]piperidin-1-yl}nicotinate
ethyl 5-cyano-6-(4-[(2-fluorobenzyl)sulfonyl]carbamoyl}piperidin-1-yl)-2-methoxynicotinate
ethyl 6-(4-{[(2-chlorobenzypsulfonyl]carbamoyl}piperidin-1-yl)-5-cyano-2-methoxynicotinate
ethyl 5-cyano-6-(4-{[(3-fluorobenzyl)sulfonyl]carbamoyl}piperidin-1-yl)-2-methoxynicotinate
ethyl 5-cyano-6-(4-{[(4-fluorobenzyl)sulfonyl]carbamoyl}piperidin-1-yl)-2-methoxynicotinate
ethyl 6-(4-{[(4-chlorobenzyl)sulfonyl]carbamoyl}piperidin-1-yl)-5-cyano-2-methoxynicotinate
ethyl 5-cyano-2-methoxy-6-[4-({[4-(trifluoromethyl)benzyl]sulfonyl}carbamoyl)piperidin-1-yl]nicotinate
ethyl 5-cyano-6-(4-{[(3,4-difluorobenzyl)sulfonyl]carbamoyl}piperidin-1-yl)-2-methoxynicotinate
ethyl 5-cyano-6-(4-{[(2,4-dichlorobenzypsulfonyl]carbamoyl}piperidin-1-yl)-2-methoxynicotinate
ethyl 5-cyano-6-(4-{[(2,4-difluorobenzyl)sulfonyl]carbamoyl}piperidin-1-yl)-2-methoxynicotinate
ethyl 6-(4-{[(2-chloro-4-fluorobenzyl)sulfonyl]carbamoyl}piperidin-1-yl)-5-cyano-2-methoxynicotinate
ethyl 6-(4-{[(4-chloro-2-fluorobenzypsulfonyl]carbamoyl}piperidin-1-yl)-5-cyano-2-methoxynicotinate
ethyl 5-cyano-6-(4-{[(2,3-difluorobenzypsulfonyl]carbamoyl}piperidin-1-yl)-2-methoxynicotinate
ethyl 5-cyano-2-methoxy-6-{3-[(phenylsulfonyl)carbamoyl]azetidin-1-yl}nicotinate
ethyl 5-cyano-6-(3-{[(2-fluorobenzyl)sulfonyl]carbamoyl}azetidin-1-yl)-2-methoxynicotinate
ethyl 6-(3-{[(2-chlorobenzypsulfonyl]carbamoyl}azetidin-1-yl)-5-cyano-2-methoxynicotinate
ethyl 5-cyano-6-(3-{[(3-fluorobenzypsulfonyl]carbamoyl}azetidin-1-yl)-2-methoxynicotinate
ethyl 5-cyano-6-(3-{[(4-fluorobenzyl)sulfonyl]carbamoyl}azetidin-1-yl)-2-methoxynicotinate
ethyl 6-(3-{[(4-chlorobenzyl)sulfonyl]carbamoyl}azetidin-1-yl)-5-cyano-2-methoxynicotinate
ethyl 5-cyano-2-methoxy-6-[3-({[4-(trifluoromethyl)benzyl]sulfonyl}carbamoyl)azetidin-1-yl]nicotinate
ethyl 5-cyano-6-(3-{[(3,4-difluorobenzyl)sulfonyl]carbamoyl}azetidin-1-yl)-2-methoxynicotinate
ethyl 5-cyano-6-(3-{[(2,4-dichlorobenzyl)sulfonyl]carbamoyl}azetidin-1-yl)-2-methoxynicotinate
ethyl 5-cyano-6-(3-{[(2,4-difluorobenzypsulfonyl]carbamoyl}azetidin-1-yl)-2-methoxynicotinate
ethyl 6-(3-{[(2-chloro-4-fluorobenzyl)sulfonyl]carbamoyl}azetidin-1-yl)-5-cyano-2-methoxynicotinate
ethyl 6-(3-{[(4-chloro-2-fluorobenzypsulfonyl]carbamoyl}azetidin-1-yl)-5-cyano-2-methoxynicotinate
ethyl 5-cyano-6-(3-{[(2,3-difluorobenzyl)sulfonyl]carbamoyl}azetidin-1-yl)-2-methoxynicotinate
ethyl 6-{3-[(benzylsulfonyl)carbamoyl]azetidin-1-yl}-5-cyano-2-(ethoxymethyl)nicotinate
ethyl 6-{4-[(benzylsulfonyl)carbamoyl]piperidin-1-yl}-5-cyano-2-(ethoxymethyl)nicotinate
ethyl 2-[(benzyloxy)methyl]-6-{3-[(benzylsulfonyl)carbamoyl]azetidin-1-yl}-5-cyanonicotinate
ethyl 2-[(benzyloxy)methyl]-6-{4-[(benzylsulfonyl)carbamoyl]piperidin-1-yl}-5-cyanonicotinate
ethyl 6-{3-[(benzylsulfonyl)carbamoyl]azetidin-1-yl}-5-cyano-2-(hydroxymethypnicotinate
ethyl 6-{3-[(benzylsulfonyl)carbamoyl]azetidin-1-yl}-5-cyano-2-ethoxynicotinate
ethyl 5-cyano-2-ethoxy-6-(3-{[(4-fluorobenzyl)sulfonyl]carbamoyl}azetidin-1-yl)nicotinate
ethyl 5-cyano-2-ethoxy-6-(3-{[(2-fluorobenzyl)sulfonyl]carbamoyl}azetidin-1-yl)nicotinate
ethyl 5-cyano-6-(3-{[(2,4-difluorobenzyl)sulfonyl]carbamoyl}azetidin-1-yl)-2-ethoxynicotinate
ethyl 6-{3-[(benzylsulfonyl)carbamoyl]azetidin-1-yl}-5-cyano-2-{[(3,4-dimethoxybenzyl)oxy]methyl}nicotinate
ethyl 5-chloro-6-(4-{[(4-chlorobenzyl)sulfonyl]carbamoyl}piperidin-1-yl)-2-(methylthio)nicotinate
ethyl 6-{4-[(benzylsulfonyl)carbamoyl]piperidin-1-yl}-5-fluoro-2-(methylthio)nicotinate
ethyl 6-{4-[(benzylsulfonyl)carbamoyl]piperidin-1-yl}-5-cyano-2-(2-methoxyethypnicotinate
ethyl 6-{4-[(benzylsulfonyl)carbamoyl]piperidin-1-yl}-2-chloro-5-fluoronicotinate
ethyl 6-{4-[(benzylsulfonyl)carbamoyl]piperidin-1-yl}-5-cyano-2-(1H-1,2,4-triazol-1-ylmethyl)nicotinate
ethyl 6-{4-[(benzylsulfonyl)carbamoyl]piperidin-1-yl}-5-cyano-2-(1H-1,2,3-triazol-1-ylmethyl)nicotinate
ethyl 6-{4-[(benzylsulfonyl)carbamoyl]piperidin-1-yl}-5-cyano-2-(1H-imidazol-1-ylmethyl)nicotinate
isopropyl 6-{4-[(benzylsulfonyl)carbamoyl]piperidin-1-yl}-2,5-dicyanonicotinate
1-(5-butyryl-3-cyano-6-methoxypyridin-2-yl)-N-[(4-fluorobenzyl)sulfonyl]piperidine-4-carboxamide
1-(5-butyryl-3-cyano-6-methoxypyridin-2-yl)-N-[(4-chlorobenzypsulfonyl]piperidine-4-carboxamide
N-(benzylsulfonyl)-1-(5-butyryl-3-cyano-6-methoxypyridin-2-yl)piperidine-4-carboxamide
ethyl 6-{4-[(benzylsulfonyl)carbamoyl]piperidin-1-yl}-5-chloro-2-(methylthio)nicotinate
isopropyl 6-(4-{[(4-chlorobenzyl)sulfonyl]carbamoyl}piperidin-1-yl)-5-cyano-2-methoxynicotinate
isopropyl 5-cyano-6-(4-{[(4-fluorobenzypsulfonyl]carbamoyl}piperidin-1-yl)-2-s methoxynicotinate
ethyl 6-{3-[(benzylsulfonyl)carbamoyl]azetidin-1-yl}-5-cyano-2-(methylthio)nicotinate
ethyl 6-{4-[(benzylsulfonyl)carbamoyl]piperidin-1-yl}-5-cyano-2-(methylthio)nicotinate
ethyl 6-{4-[(benzylsulfonyl)carbamoyl]piperidin-1-yl}-2,5-dichloronicotinate
isopropyl 6-{4-[(benzylsulfonyl)carbamoyl]piperidin-1-yl}-5-cyano-2-methoxynicotinate
N-(benzylsulfonyl)-1-[3-cyano-6-(methylthio)-5-pentanoylpyridin-2-yl]piperidine-4-carboxamide
1-[3-cyano-6-(methylthio)-5-pentanoylpyridin-2-yl]-N-[(4-methoxybenzyl)sulfonyl]piperidine-4-carboxamide;
and pharmaceutically acceptable salts thereof.

Processes

The following processes together with the intermediates are provided as a further feature of the present invention.

Compounds of formula (I) may be prepared by the following processes a1-a10;

a1) Compounds of formula (I) in which R₁, R₂, R₄, B, R₁₄, R₁₅, R^{c} and R^{d} are defined as in formula (I) above, X is a single bond or a carbon, can be formed by reacting a compound of formula (II), in which R₁, R₂, R₄, B, R₁₄, and R₁₅ are defined

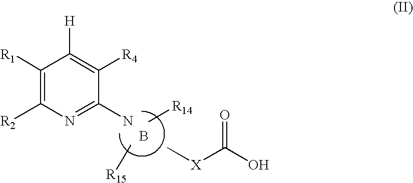

as in formula (I) above, X is a single bond or a carbon, with a compound of formula (III) in which R^{c} and R^{d} are defined as in formula (I) above.

H₂NSO₂—R^{c}—R^{d} (III)

The reaction is generally carried out in an inert organic solvent such as dichloromethane at ambient temperature. The reaction may be carried out using standard conditions or in the presence of TBTU, EDCI, PyBrop or the combination of EDCI and HOBt. Optionally, the reaction may be carried out in the presence of an organic base such as triethylamine or DIPEA.

a2) Compounds of formula (I) in which R₁, R₂, R₄, B, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (IV), in which R₁, R₂, R₄, B, R₁₄, and R₁₅ are defined as in formula (I) above and X is a nitrogen, (—CH₂—NH₂) or a hydrogen that is connected to a nitrogen which is a member of the B-ring, with a compound of the general

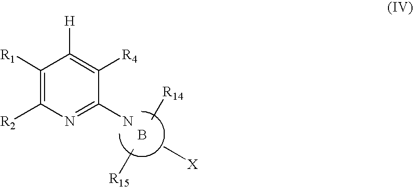

formula (III) which is defined as above.

The reaction is generally carried out in an inert solvent such as DCM. The reaction may be carried out in the presence of CDI. Optionally, the reaction may be carried out in the presence of an organic base such as triethylamine, DBU or DIPEA.

a3) Compounds of formula (I) in which R₁, R₂, R₄, B, R₁₄, R₁₅, R^{c} and R^{d} are defined as in formula (I) above X is a nitrogen, (—CH₂—NH—) or a single bond connected to a nitrogen which is a member of the B ring, can be formed by reacting a compound of formula (IV) which is defined in a2) above, with a compound of formula (V)

O═C═N—SO₂—R^{c}R^{d} (V)

in which R^{c} and R^{d} are defined as in formula (I) above.

The reaction is generally carried out in an inert solvent such as THF. Optionally, the reaction may be carried out in the presence of an organic base such as triethylamine or DIPEA.

a4) Compounds of formula (I) in which R₁, R₂, R₄, B, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (IV) which is defined in above, with a compound of formula (VI),

R^{d}R^{c}—SO₂NH—COOCH₂CCl₃ (VI)

in which R^{c} and R^{d} are defined as in formula (I) above. The reaction is generally carried out in an inert solvent such as DMA. Optionally, the reaction may be carried out in the presence of an organic base such as triethylamine or DIPEA.

a5) Compounds of formula (I) may also be prepared by reacting a compound of formula (VII) in which R₁, R₂, and R₄ are defined as in formula (I) above and L is a suitable leaving group, such as chloro, bromo, iodo, fluoro, triflate (OTf) mesylate (OMs) or tosylate (OTs),

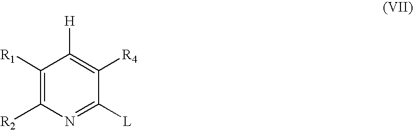

with a compound of the general formula (VIII) in which B, X, R₁₄, R₁₅, R^{c} and R^{d} are defined as in formula (I) above.

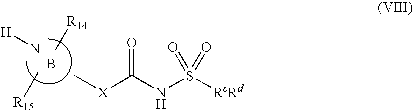

The reaction is generally carried out in an inert solvent such as DMA. Optionally, the reaction may be carried out in the presence of an organic base such as triethylamine or DIPEA.

The reaction is generally carried out at elevated temperatures using standard equipment or in a single-node microwave oven.

For some compounds, it is advantageous to carry out the reaction in ethanol in the presence of an organic base such as triethylamine.

a6) Compounds of formula (I) where R₁ represents R₆OC(O) and R₂, R₄, B, R₆, R₁₄, R₁₅, X, R^{c} and R^{d} are defined as in formula (I) above, can be transesterified using standard procedures or by reacting with R_{6′}—O⁻Li⁺ reagent, to become another compound of the general formula (I) wherein R₁ becomes R_{6′}OC(O).

a7) A compound of formula (I) in which R₁, R₂, R₄, B, R₁₄, R₁₅, and R^{d} are defined as in formula (I) above and R^{c} represents imino (—NH—) or (C₁-C₄)alkylimino in which the imino group could be substituted using standard conditions or using an alkylating agent like L-R₁₉, in which R₁₉ is defined as in formula (I) above and L is a leaving group exemplified by chloro, bromo, iodo, triflate (OTf) or tosylate (OTs), to give compounds of formula (I) in which R₁, R₂, R₄, B, R₁₄, R₁₅, and R^{d} are defined as in formula (I) above and R^{c} represents N-substituted imino (—NR₁₉—) or N-substituted (C₁-C₄)alkylimino (—N(R₁₉)—((C₁-C₄)alkyl), optionally in the presence of a strong base such as NaH.

a8) Compounds of formula (I) in which R₁ is R₆OC(O) and R₄, B, R₆, R₁₄, R₁₅, X, R^{c} and R^{d} are as defined in formula (I) above, R₂ is an (C₁-C₁₂)alkoxy group defined as in formula (I) above may be prepared by reacting a compound of formula (IX)

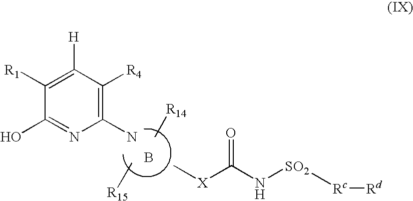

in which R₁ is R₆OC(O) and R₄, B, R₆, R₁₄, R₁₅, X, R^{c} and R^{d} are as defined in formula (I) above with a compound of formula (X)

L-R_{2′} (X)

in which R2′ is an (C₁-C₁₂)alkyl defined as in formula (I) above and L is a leaving group such as chloro, bromo, iodo, triflate (OTf) or tosylate (OTs).

The reaction may be carried out in an inert organic solvent such as DMA, THF or CH₃CN. The reaction may be carried out using standard conditions or in the presence of a suitable base such as sodium hydride, DIPEA or silver carbonate or potassium carbonate. Preferentially silvercarbonate is used.

The reaction may be carried out at ambient temperature or at elevated temperatures using standard equipment or a single node microwave oven.

a9) Compounds of formula (I) in which R₁ is R₆OC(O) and R₄, B, R₆, R₁₄, R₁₅, X, R^{c} and R^{d} are as defined in formula (I) above, R₂ is a cyano group, an (C₁-C₁₂)alkoxy group or an (C₁-C₁₂)alkylthio group defined as in formula (I) above can be prepared by s reacting a compound of formula (XI)

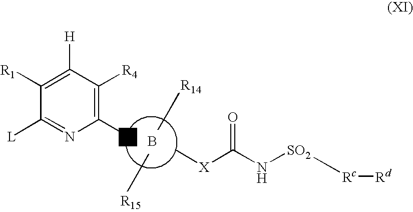

in which R₁ is R₆OC(O) and R₄, B, R₆, R₁₄, R₁₅, X, R^{c} and R^{d} are as defined in formula (I) above and L is a suitable leaving group such as Cl, Br, I or triflate (OTf) with sodium cyanide, the corresponding (C₁-C₁₂)alcohol and (C₁-C₁₂)alkylthiol respectively.

The reaction may be performed using standard conditions in the precence of a palladium catalyst such as or Pd(PPh₃)₄ or Pd₂(dba)₃ in combination with a suitable phosphine ligand such as PPh₃ or XANTPHOS. The reaction may be carried out in an inert solvent such as DCM, THF or dioxane optionally in the precence of a base such as DIPEA. The reaction may be carried out at ambient temperature or at elevated temperatures using standard equipment or a single node microwave oven.

a10) Compounds of formula (I) in which R₁ is R₆OC(O) and R₄, B, R₆, R₁₄, R₁₅, X, R^{c} and R^{d} are as defined in formula (I) above, R₂ is a substituted C₁-alkyl group defined as in formula (I) above can be prepared by reacting a compound of formula (XII)

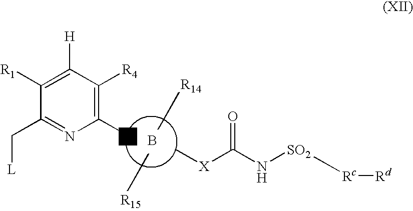

in which R₁ is R₆OC(O) and R₄, B, R₆, R₁₄, R₁₅, X, R^{c} and R^{d} are as defined in formula (I) above and L is a suitable leaving group such as Cl, Br, I, triflate (OTf) or tosylate (OTs) with the corresponding nucleophile to give the substituted C₁-alkyl group described for R₂ above.

The reaction is carried out using standard conditions in an inert solvent such as EtOH, DMF or acetone.

Preferentially the reaction is carried out in the precence of a base such as DIPEA, TEA or Cs₂CO₃.

Optionally the reaction is performed in the precence of sodium iodide.

The reaction may be carried out at ambient temperature or at elevated temperatures using standard equipment or a single node microwave oven.

The intermediates referred to above may be prepared by, for example, the methods/processes outlined below.

b) The compounds of formula (II) in which R₁, R₂, R₄, B, R₁₄, and R₁₅ are defined as in formula (I) above, X is a single bond or a carbon, may be prepared by reacting a compound of formula (VII) defined above and L is a suitable leaving group (such as fluoro, chloro, bromo, iodo, triflate (OTf) mesylate (OMs) or tosylate (OTs)), with a compound of the general formula (XIII),

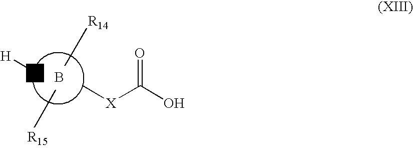

in which B, R₁₄, R₁₅ are defined as in formula (I) above and X is a single bond or a carbon.

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

c) Compounds of formula (IV) which are defined as above may be prepared by reacting the corresponding compound of formula (VII) which is defined above, with a compound of formula (XIV) in which B, R₁₄, R₁₅ are defined as in formula (I) above, X is a nitrogen, (—CH₂—NH—) or a single bond connected to a nitrogen which is a member of the B ring.

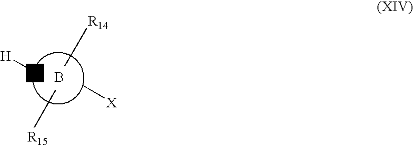

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the prescence of an organic base such as TEA or DIPEA.

d) Synthesis of compounds of the general formula (XV),

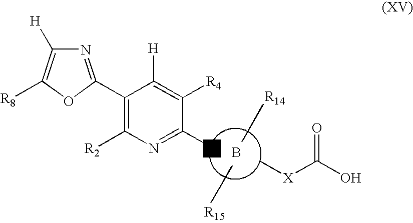

in which R₂, R₄, B, R₈, R₁₄ and R₁₅ are defined as in formula (I) above and X is a carbon or a single bond comprises the below steps. (d1-d5)

d1) Reacting the corresponding compounds of the general formula (XIII) which is defined as above with a compound of the general formula (XVI)

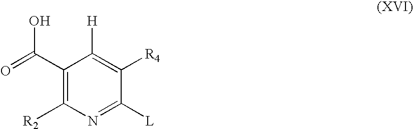

in which R₂ and R₄ are defined as in formula (I) above, and L is a suitable leaving group, such as chloro, bromo, iodo, triflate (OTf), mesylate (OMs) or tosylate (OTs), to give a compound of formula (XVII).

The reactions are carried out at elevated temperatures using standard equipment or a single-node microwave oven. Optionally the reaction may be carried out in the prescence of an organic base such as TEA or DIPEA.

d2) The compounds of formula (XVII) can then be reacted

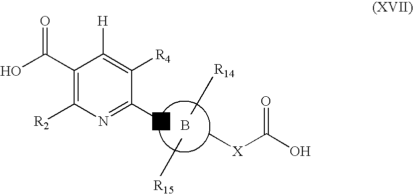

with a compound of the general formula (XVIII),

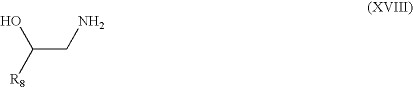

in which R₈ is defined as in formula (I) above, to give compounds of the general formula (XIX). The reactions may be carried out using standard conditions or in the prescence of EDCI or the combination of EDCI and HOBt. Optionally the reaction may be carried out in the prescence of an organic base such as TEA or DIPEA.

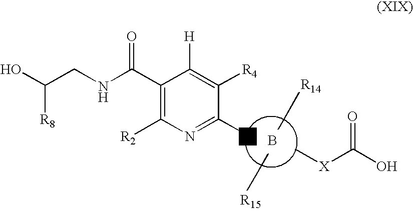

d3) This compound (XIX) can then be transformed to a compound of the general formula (XX)

d4) The preparation of compounds with the general formula (XX),

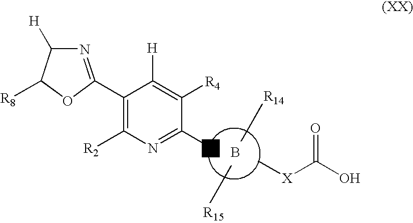

in which R₂, R₄, B, R₈, R₁₄ and R₁₅ are defined as in formula (I) above and X is a carbon or a single bond using known methods or a known reagent such as methanesulfonyl chloride. Optionally the reaction may be carried out in the prescence of an organic base such as TEA.

d5) a compound of the general formula (XV) as defined above can be made by oxidizing the corresponding compound of the general formula (XX) using a known oxidation reagent such as DDQ.

e) The preparation of compounds of the general formula (XV) also comprises the steps (e1-e7) below;

e1) Reacting a compound the general formula (XXI),

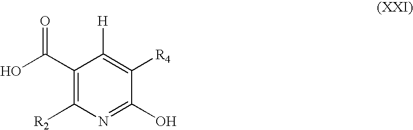

in which R₂ and R₄ are defined as in formula (I) above, with a compound of the general formula (XXII), in which R₈ is defined as in formula (I) above,

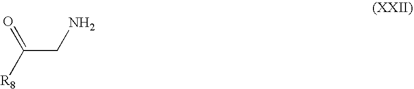

using standard conditions or in the prescence of EDCI or the combination of EDCI and

HOBt. Optionally the reaction may be carried out in the prescence of an organic base such as TEA. This reaction gives a compound of the general formula (XXIII).

e2) The compound of the general formula (XXIII) obtained

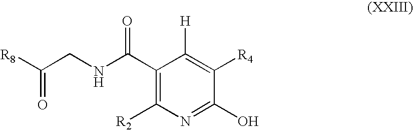

can then be transformed to a compound of the general formula (XXIV), in which R₂, R₄ and R₈ are defined as in formula (I) above, using known techniques or using a known reagent such as POCl₃ or in the presence of (Methoxycarbonylsulfamoyl)triethylammonium hydroxide (Burgess reagent).

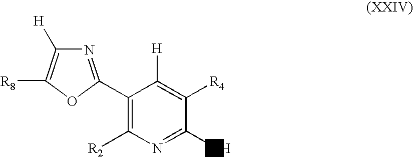

The preparation of compounds of the general formula (XXIV) which is defined as above can also comprise the steps (e3-e5) below;

e3) Reacting a compound of the general formula (XXI) above

with a compound of the general formula (XVIII), defined as above, to give a compound of the formula (XXV). The reaction is generally carried out in DCM at ambient temperature. The reaction may be carried out using standard conditions or in the presence of EDCI or the combination of EDCI and HOBt. Optionally the reaction may be carried out in the prescence of an organic base such as TEA or DIPEA.

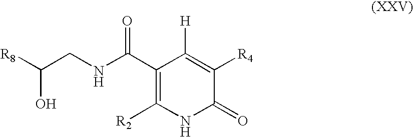

e4) The compound of formula (XXV) can be transformed to a compound (XXIII) using standard conditions or an oxidizing agent such as the mixture of oxalylchloride and DMSO.

e5) The compound of formula (XXIII) can then be transformed into a compound of the general formula (XXIV), using standard conditions or in the presence of (Methoxycarbonylsulfamoyl)triethylammonium hydroxide (Burgess reagent). The reaction is generally performed in an inert solvent such as THF. The reaction is carried out at elevated temperatures using standard equipment or a single-node microwave oven.

e6) A compound of the general formula (XXIV) can then be transformed to a compound of the general formula (XXVI),

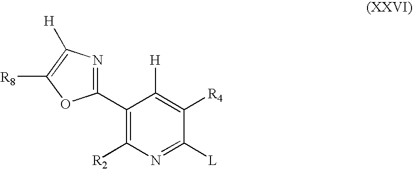

in which R₂, R₄, R₈ are defined as in formula (I) above and L is a sufficient leaving group, such as chloro, bromo, iodo, triflate (OTf), mesylate (OMs) or tosylate (OTs), using a known techniques or a reagent such as oxalyl chloride or thionyl chloride.

e7) The compound of formula (XXVI) can then be reacted with a compound of the general formula (XIII), which is defined as above, to give a compound of the general formula (XV), defined as above. The reactions are carried out at elevated temperatures using standard equipment or a single-node microwave oven. Optionally the reactions may be carried out in the prescence of an organic base such as TEA or DIPEA.

f) Preparation of Compounds of the general formula (XXVII),

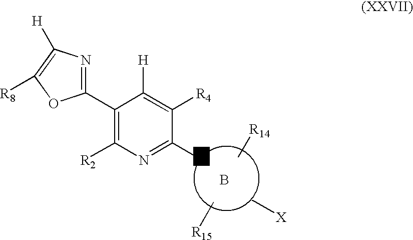

in which R₂, R₄, B, R₈, R₁₄ and R₁₅ are defined as in formula (I) above, X is a nitrogen, (—CH₂—NH—) or a single bond connected to a nitrogen which is a member of the B ring, comprises the below steps. (f1-f4)

f1) Reacting a compound of the general formula (XIV) which is defined as above with a compound of the general formula (XVI) which is defined as above, to give a compound of the general formula (XXVIII).

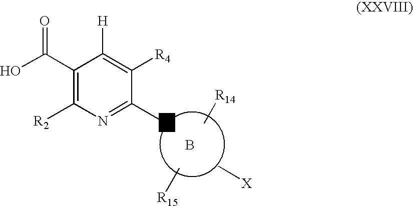

The reactions are carried out at elevated temperatures using standard equipment or a single-node microwave oven. Optionally the reaction may be carried out in the prescence of an organic base such as TEA or DIPEA.

f2) The compound of formula (XXVIII) can be reacted with a compound of formula (XVIII), which is defined as above, to give compounds of the general formula (xxix). The reactions are carried out using standard conditions or in the prescence of EDCI or the combination of EDCI and HOBt. Optionally the reactions may be carried out in the prescence of an organic base such as TEA or DIPEA.

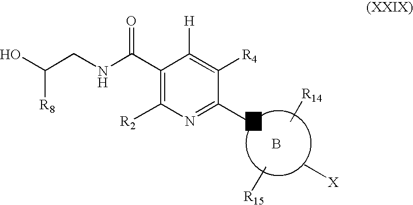

f3) This compound can then be transformed to a compound of the general formula (XXX) in which R₂, R₄, B, R₈, R₁₄ and R₁₅, are defined as in formula (I) above,

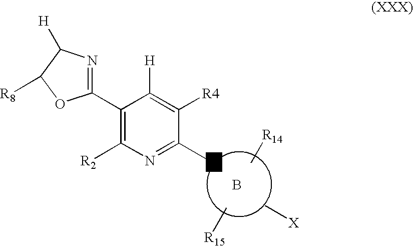

X is a nitrogen, (—CH₂—NH—) or a hydrogen connected to a nitrogen which is a member of the B ring, using known methods or a sufficient reagent such as methanesulfonyl chloride. Optionally the reaction may be carried out in the prescence of an organic base such as TEA.

f4) (XXVII) can then prepared by oxidizing a compound of the general formula (XXX), which is defined as above. The reaction can be performed using standard conditions or a reagent like DDQ.

Compounds of the general formula (II), in which R₁ is R₇C(O) and R₂, R₄, R₇, B, R₁₄ and R₁₅ are defined as in formula (I) above, X is a single bond or a carbon atom comprises the following steps (g1-g2):

g1) Reacting a compound of the general formula (XVII), described above, with N,O-dimethylhydroxylamine. The reaction can be performed using known reagents like CDI, EDCI or the combination of EDCI and HOBt to give a compound of the general formula (XXXI).

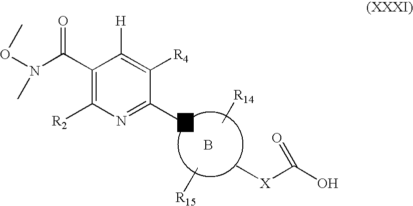

g2) Reacting compounds of the general formula (XXXI), defined as above, with a reagent of the general formula R₇—MgX′, in which R₇ is defined as in formula (I) above and X′ is a halogen, or a reagent of the formula R₇-M, in which M is a metal examplified by Zn and Li.

Compounds of the general formula (II), in which R₁ is R₇C(O) and R₂, R₄, R₇, B, R₁₄ and R₁₅ are defined as in formula (I) above, X is a single bond or a carbon atom also comprises the following steps (g3-g4):

g3) Reacting compounds of general formula LI

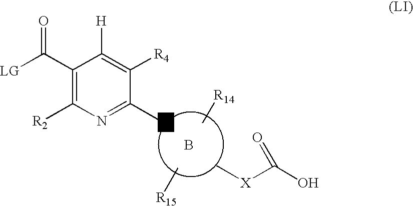

wherein R₂, R₄, B, R₁₄ and R₁₅ is as defined in formula (I) above, X is a single bond or a carbonatom and LG is a leaving group such as Cl or F with a reagent of general formula R₇—MgX′, in which R₇ is defined as in formula (I) above.

The reaction is carried out using standard conditions in an inert solvent such as THF catalyzed by ferric acetylacetonate or other suitable ferric salts such as for example FeCl₃.

The reaction may be performed at ambient temperature or preferentially at lower temperatures for example in the range of —78° C. and 0° C.

(See for example Fürstner A et al, J. Org Chem, 2004, pp 3943-3949)

g4) Compounds of general formula (LI) above can by prepared by reacting a compound of general formula (XVII) defined as above using standard conditions or with a chlorinating reagent such as oxalyl chloride, thionyl chloride or POCl₃ (e.g. when LG is Cl). Advantageously dimethylformamide may be used as catalyst.

The reaction can also be performed using standard conditions with cyanuric fluoride preferentially in the precence of pyridine (e.g. when LG is F)

The reaction may be performed in an inert solvent such as DCM or toluene. The reaction is carried out at ambient temperature or at elevated temperatures.

Compounds of the general formula (II), in which R₁ is R₇C(O) (this is a special case for all compounds which contains a R₇ group containing a CH₂ group next to the carbonyl in R₁ referred to below as R_{7′}—CH₂) and R₂, R₄, R₇, B, R₁₄ and R₁₅ are defined as in formula (I) above, X is a single bond or a carbon atom also comprises the following steps (g5-g7):

g5) By double decarboxylation of a compound of general formula (LII)

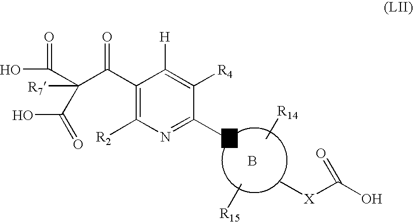

The reaction is generally carried at elevated temperature using standard equipment. Preferentially the reaction is carried out under acidic conditions in an inert solvent such as MeCN or THF.

g6) Compounds of the formula (LII) above can be prepared by reaction of a compound of formula (LI) with a compound of formula (LIII)

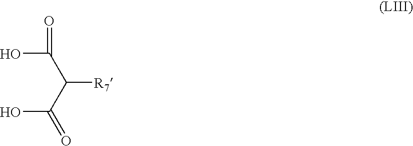

The reaction is carried out in an inert solvent such as THF at ambient temperature in the presence of a suitable base such as sodium pentoxide or NaH.

(For similar chemistry see, Asish D. et al, J. Chem. Soc. Perkin Treans. I, 1989, pp 603-607 and Rathke, M et al, J. Org. Chem. 1985, pp 2622-24).

g7) Compounds of the general formula (II), in which R₁ is R₁₆SC(O) and R₂, R₄, B, R₁₄ and R₁₅ are defined as in formula (I) above, X is a single bond or a carbon atom can be made by reacting a compound of formula (XVII) with CDI and R₁₆SH or R₁₆SNa. The reaction is carried out in an inert solvent such a as THF or DCM at ambient temperature or at elevated temperatures.

Compounds of the general formula (IV), in which R₁ is R₇C(O) and R₂, R₄, R₇, B, R₁₄ and R₁₅ are defined as in formula (I) above, X is a nitrogen, (—CH₂—NH—) or a single is bond connected to a nitrogen which is a member of the B ring, comprises the following steps (h1-h2).

h1) Reacting a compound of the general formula (XXVIII), defined as above, with N,O-dimethylhydroxylamine. The reaction can be performed using known reagents like CDI, EDCI or the combination of EDCI and HOBt to give a compound of the general formula (XXXII).

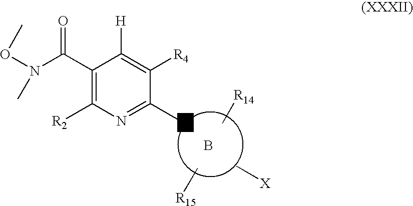

h2) A compound of the general formula (XXXII), which is defined as above can be reacted with a reagent of the general formula R₇—MgX, in which R₇ is defined as in formula (I) above and X is a halogen, or a reagent of the formula R₇-M, in which M is a metal exemplified by Zn and Li.

Compounds of the general formula (IV), in which R₁ is R₇C(O) and R₂, R₄, R₇, B, R₁₄ and R₁₅ are defined as in formula (I) above, X is a nitrogen, (—CH₂—NH—) or a single bond connected to a nitrogen which is a member of the B ring, also comprises the following steps (h3-h4).

h3) Reacting compounds of general formula LIV

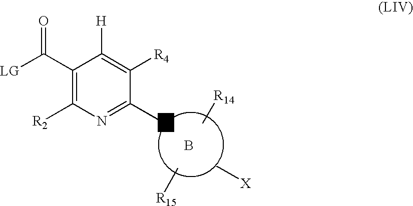

wherein R₂, R₄, B, R₁₄ and R₁₅ is as defined in formula (I) above, X is a nitrogen, (—CH₂—NH—) or a single bond connected to a nitrogen which is a member of the B ring and LG is a leaving group such as Cl or F with a reagent of general formula R₇—MgX′, in which R₇ is defined as in formula (I) above.

The reaction is carried out using standard conditions in an inert solvent such as THF catalyzed by ferric acetylacetonate or other suitable ferric salts.

The reaction may be performed at ambient temperature or preferentially at lower temperatures for example in the range of —78° C. and 0° C.

(See for example Fürstner A et al, J. Org Chem, 2004, pp 3943-3949)

h4) Compounds of general formula (LIV) above can by prepared by reacting a compound of general formula (XXVIII) defined as above using standard conditions or with a chlorinating reagent such as oxalyl chloride, thionyl chloride or POCl₃ (e.g. when LG is Cl). Advantageously dimethylformamide may be used as catalyst.

The reaction can also be performed using standard conditions with cyanuric fluoride preferentially in the precence of pyridine (e.g. when LG is F)

The reaction may be performed in an inert solvent such as DCM or toluene. The reaction is carried out at ambient temperature or at elevated temperatures.

Compounds of the general formula (IV), in which R₁ is R₇C(O) (this is a special case for all compounds which contains a R₇ group containing a CH₂ group next to the carbonyl in R₁ referred to below as R₇′—CH₂) and R₂, R₄, B, R₁₄ and R₁₅ are defined as in formula (I) above, X is a nitrogen, (—CH₂—NH—) or a single bond connected to a nitrogen which is a member of the B ring, also comprises the following steps (h5-h6).

h5) By double decarboxylation of a compound of general formula (LV)

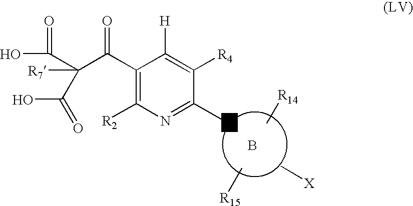

The reaction is generally carried at elevated temperature using standard equipment. Preferentially the reaction is carried out under acidic conditions in an inert solvent such as MeCN or THF.

h6) Compounds of the formula (LV) above can be prepared by reaction of a compound of formula (LIV) with a compound of formula (LIII)

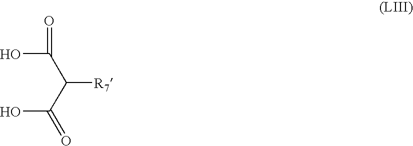

The reaction is carried out in an inert solvent such as THF at ambient temperature in the presence of a suitable base such as sodium pentoxide or NaH.

(For similar chemistry see, Asish D. et al, J. Chem. Soc. Perkin Treans. I, 1989, pp 603-607 and Rathke, M et al, J. Org. Chem. 1985, pp 2622-24).

h7) Compounds of the general formula (IV), in which R₁ is R₁₆SC(O) and R₂, R₄, B, R₁₄ and R₁₅ are defined as in formula (I) above, X is a nitrogen, (—CH₂—NH—) or a single bond connected to a nitrogen which is a member of the B ring, can be made by reacting a compound of formula (XXVIII) with CDI and R₁₆SH or R₁₆SNa.

The reaction is carried out in an inert solvent such a as THF or DCM at ambient temperature or at elevated temperatures.

Compounds of the general formula (VIII) can be formed in one of the processes (i1-i4). The compounds of formula (VIII) are advantageously isolated as a zwitterion. A ring nitrogen of compounds of formula (XIII) and (XIV) used in the below steps may be protected by a protective group such as t-butyloxycarbonyl.

i1) Compounds of the general formula (VIII) in which B, R₁₄, Rᵢₛ, R^{c} and R^{d} are defined as in formula (I) above, X is a single bond or a carbon, may be formed by reacting a compound of formula (XIII) with a compound of formula (III). The reaction is generally carried out in an inert organic solvent such as dichloromethane at ambient temperature. The reaction may be carried out using standard conditions or in the presence of EDCI or the combination of EDCI and HOBt. Optionally, the reaction may be carried out in the presence of an organic base such as triethylamine or DIPEA.

i2) Compounds of the general formula (VIII) in which B, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (XIV) defined as above with a compound of formula (V), defined as above. The reaction is generally carried out in an inert solvent such as THF. The reaction may also be carried out in the presence of an organic base such as triethylamine or DIPEA.

i3) Compounds of the general formula (VIII) in which B, R₁₄, R₁₅, R^{c} and R^{d} defined as in formula (I) above, X is a nitrogen, (—CH₂—NH—) or a single bond connected to a nitrogen which is a member of the B ring, can also be formed by reacting a compound of formula (XIV) with a compound of formula (VI) which is defined as above. The reaction is generally carried out in a solvent such as DMA. This reaction may also be carried out in the presence of an organic base such as triethylamine or DIPEA

i4) A compound of formula (VIII) which is protected with t-butoxy carbonyl may be transformed into a compound without the protective group using standard procedures or a reagent such as HCl or TFA.

j1) Compounds of the general formula (VII) which are defined as above can be formed by reacting a compound of formula (XXXEII) using standard conditions or with a chlorinating reagent such as oxalyl chloride, thionyl chloride or POCl₃. Advantageously dimethylformamide may be used. The reaction may be performed in an inert solvent such as DCM. Advantageously the inert solvent is toluene.

The reaction may also be carried out with methyl sulfonyl chloride in the presence of a base, such as DIPEA, in an inert solvent such as DCM.

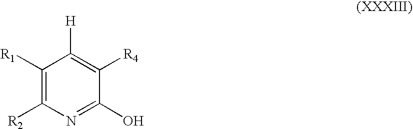

j2a) Compounds of the general formula (VII) in which R₁ is R₁₆S(CO), L is Cl, and R₂, and R₄ are as defined in Formula I may be formed by reacting a compound of formula L

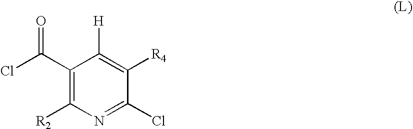

in which R₂ and R₄ are defined as in formula (I) with R₁₆SH or R₁₆SNa, wherein R₁₆ is defined as in formula (I), in an inert organic solvent such as DCM or THF, Optionally the reaction is carried out in the presence of an organic base such as DIPEA or TEA.

j2b) Compounds of the general formula (L) can be formed by reacting a compound of formula (XXI) defined as above using standard conditions or with a chlorinating reagent such as oxalyl chloride, thionyl chloride or POCl₃. Advantageously dimethylformamide may be used as catalyst. The reaction may be performed in an inert solvent such as DCM or toluene. The reaction is carried out at ambient temperature or at elevated temperatures.

l) Preparation of compounds of the general formula (XXI) which is defined as above comprises the following steps (l₁-l₃);

l1) Reacting a compound of the formula (XXXIV), in which R₂ and R₆ are defined as in formula (I) above with dimethoxy-N,N-dimethylmethaneamine to form a

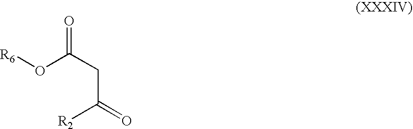

compound of formula (XXXV).

l2) This compound (XXXV) can then be reacted further with a compound of the

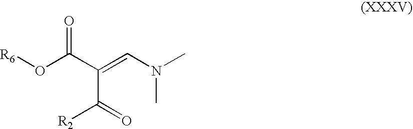

general formula R₄CH₂C(O)NH₂, in which R₄ is defined as in formula (I) above to give a compound of the general formula (XXXVI). The reaction is generally performed in an inert solvent such as ethanol, optionally in the presence of a strong base such as sodium ethoxide.

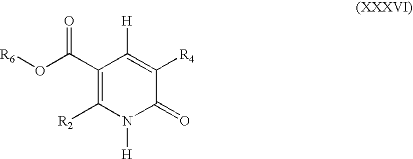

(l3) A compound of the general formula (XXXVI) can then be transformed to a compound of the general formula (XXI). The reaction is generally performed in a protic solvent such as water together with a co-solvent such as THF or methanol. The reaction can be performed using standard reagents or in the presence of LiOH, NaOH or KOH.

m) Compounds of the general formula (IX) wherein R₁₄, R₁₅, B, X, R^{c} and R^{d} are defined as in formula (I) R₁ is R₆OC(O) and R₄ is CN may be prepared by the following steps m1-m9 below

m1) Reacting a compound of the general formula (XXXVII)

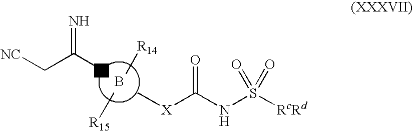

where B, R₁₄, R₁₅, X, R^{c} and R^{d} are as defined in formula (I) above with a compound of formula (XXXVIII)

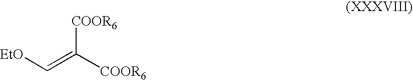

The reaction is generally carried out in an inert organic solvent such as EtOH or DMSO.

The reaction is carried out at ambient temperature or at elevated temperatures using to standard equipment or a single node microwave oven.

m2) Compounds of the general formula (XXXVIII) defined above can be prepared by reacting a compound of the general formula (VIII) as defined above with a compound of formula (XXXIX)

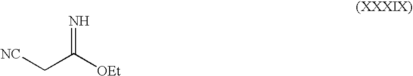

using essentially the same procedure as described in [Macconi, A et. Al, J. Heterocyclic chemistry, 26, p. 1859 (1989)].

m3) Compounds of general formula (IX) above wherein B, R₁₄, R₁₅, R^{c} and R^{d} are defined as in formula (I), R₁ is R₆OC(O) , R₄ is CN and X is a single bond or a carbon atom may be prepared by reacting a compound of formula (XXXX)

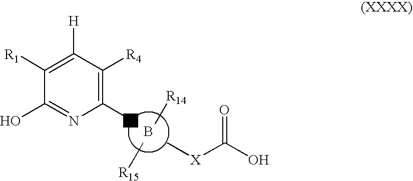

with a compound of formula (III) defined as above.

The reaction is generally carried out in an inert organic solvent such as dichloromethane at ambient temperature. The reaction may be carried out using standard conditions or in the presence of TBTU, EDCI, PyBrop or the combination of EDCI and HOBt. Optionally, the reaction may be carried out in the presence of an organic base such as triethylamine or DIPEA.

m4) Compounds of general formula (XXXX) may be prepared by reacting a compound of general formula (XXXXI)

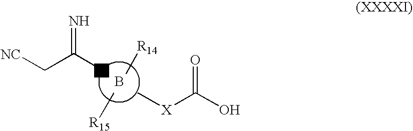

wherein R₁₄, R₁₅, and B is defined as in formula (I) and X is a single bond or a carbon atom with a compound of formula (XXXVIII) defined as above.

The reaction is generally carried out in an inert organic solvent such as EtOH or DMSO.

The reaction is carried out at ambient temperature or at elevated temperatures using standard equipment or a single node microwave oven.

m5) Compounds of the general formula (XXXXI) defined above can be prepared by reacting a compound of the general formula (XIII) as defined above with a compound of formula (XXXIX) using essentially the same procedure as described in [Macconi, A et. Al., J. Heterocyclic chemistry, 26, p. 1859 (1989)].

m6) Compounds of general formula (IX) above wherein B, R₁₄, R₁₅, R^{c} and R^{d} are defined as in formula (I), R₁ is R₆OC(O), R₄ is CN and X is a nitrogen, (—CH₂—NH—) or a single bond connected to a nitrogen which is a member of the B ring may be prepared by reacting a compound of formula (XXXXII)

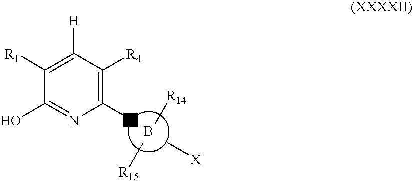

with a compound of formula (III) defined as above.

The reaction is generally carried out in an inert solvent such as DCM. The reaction may be carried out in the presence of CDI. Optionally, the reaction may be carried out in the presence of an organic base such as triethylamine, DBU or DIPEA.

m7) Compounds of general formula (IX) above wherein R₁₄, R₁₅, R^{c} and R^{d} are defined as in formula (I), R₁ is R₆OC(O), R₄ is CN and X is a nitrogen, (—CH₂—NH—) or a is single bond connected to a nitrogen which is a member of the B ring may be prepared by reacting a compound of formula (XXXXII) with a compound of general formula (V) as defined above.

The reaction is generally carried out in an inert solvent such as THF. Optionally, the reaction may be carried out in the presence of an organic base such as triethylamine or DIPEA.

m8) Compounds of general formula (IX) above wherein B, R₁₄, R₁₅, R^{c} and R^{d} are defined as in formula (I), R₁ is R₆OC(O), R₄ is CN and X is a nitrogen, (—CH₂—NH—) or a single bond connected to a nitrogen which is a member of the B ring may be prepared by reacting a compound of formula (XXXXII) with a compound of general formula (VI) as defined above.

The reaction is generally carried out in an inert solvent such as DMA. Optionally, the reaction may be carried out in the presence of an organic base such as triethylamine or DIPEA.

m9) Compounds of general formula ( )XXXII) above may be prepared by essentially the same procedure described in steps m4)-m5) above from a compound of formula (XIV)

n1) Compounds of the general formula (XII) above in which R₁ is R₆OC(O) R₄, is CN and B, R₆, R₁₄, R₁₅, X, R^{c} and R^{d} are as defined in formula (I) above may be prepared by reacting a compound of formula (XXXXIII)

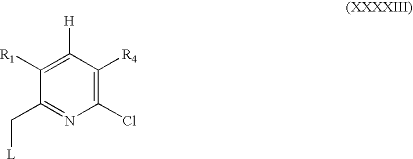

wherein R₁ is R₆OC(O) R₄ is CN and L is a leaving group such as Cl, with a compound of formula (VIII) defined as above.

The reaction may be carried out in an inert solvent such as DMA or EtOH. Optionally, the reaction may be carried out in the presence of an organic base such as triethylamine or DIPEA.

The reaction is generally carried out at elevated temperatures using standard equipment or in a single-node microwave oven.

For some compounds, it is advantageous to carry out the reaction in ethanol in the presence of an organic base such as triethylamine.

n2) Compounds of general formula (XXXXIII) as defined above may be prepared by reacting a compound of formula (XXXXIV), wherein

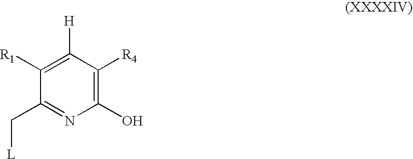

R₁ is R₆OC(O), R₄ is CN and L is a leaving group such as for example Cl, with a chlorinating reagent such as oxalyl chloride, thionyl chloride or POCl₃. Advantageously dimethylformamide may be used. The reaction may be performed in an inert solvent such as DCM.

The reaction is generally carried out at elevated temperatures.

n3) Compounds of the general formula (XXXXIV) as defined above may be prepared by reacting a compound of general formula (XXXXV), wherein R₆ is as defined in formula (I),

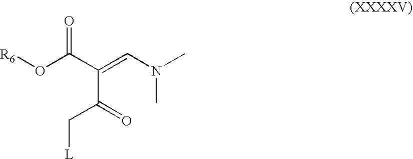

with NC—CH₂C(O)NH₂.

The reaction is generally performed in an inert solvent such as ethanol, optionally in the presence of a strong base such as sodium ethoxide.

o1) Compounds of general formula (II) wherein B, R₁₄, R₁₅, R^{c} and R^{d} are defined as in formula (I), R₁ is R₆OC(O), R₄ is CN, R₂ is an (C₁-C₁₂)alkoxy group and X is a single bond or a carbon atom may be prepared by reacting a compound of formula (XXXX) as defined above, with a compound of formula (X)

L-R_{2′} (X)

in which R2′ is an (C₁-C₁₂)alkyl defined as in formula (I) above and L is a leaving group such as chloro, bromo, iodo, triflate (OTf) or tosylate (OTs).

The reaction may be carried out in an inert organic solvent such as DMA, THF or CH₃CN. The reaction may be carried out using standard conditions or in the presence of a suitable base such as sodium hydride, DIPEA or silver carbonate or potassium carbonate. Preferentially silvercarbonate is used.

The reaction may be carried out at ambient temperature or at elevated temperatures using standard equipment or a single node microwave oven.

o2) Compounds of general formula (IV), wherein B, R₁₄, R₁₅, R^{c} and R^{d} are defined as in formula (I), R₁ is R₆OC(O), R₄ is CN, R₂ is an (C₁-C₁₂)alkoxy group and X is a nitrogen atom, (—CH₂—NH—) or a single bond connected to a nitrogen atom which is a member of the B ring may be prepared by reacting a compound of formula (XXXXII) as defined above, with a compound of formula (X)

L-R_{2′} (X)

in which R2′ is an (C₁-C₁₂)alkyl defined as in formula (I) above and L is a leaving group such as chloro, bromo, iodo, triflate (OTf) or tosylate (OTs).

The reaction may be carried out in an inert organic solvent such as DMA, THF or CH₃CN. The reaction may be carried out using standard conditions or in the presence of a suitable base such as sodium hydride, DIPEA or silver carbonate or potassium carbonate. Preferentially silvercarbonate is used.

The reaction may be carried out at ambient temperature or at elevated temperatures using standard equipment or a single node microwave oven.

p) Compounds of general formula (XII) as defined above may be prepared by reacting a compound of formula (IX) with a halogenating reagent, such as thionylchloride, POCl₃ or oxalyl chloride. Optionally the reaction is performed in the presence of DMF.

The reaction may also be carried out in an inert solvent, such as DCM, using trifluoromethanesulfonic anhydride, optionally in the presence of an organic base such as TEA or DIPEA at or below r.t.

q) The preparation of compounds of the general formula (XXXXVI), in which B, R₁₄ and R₁₅ are defined as for formula (I) with the exception that R₁₄ is connected to the same atom as X, and X is defined as a single bond, comprises the below step;

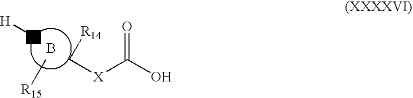

q1) Reacting the corresponding (XXXXVII) with R₁₄-L, wherein L is a suitable leaving group, such as chloro, bromo, iodo,

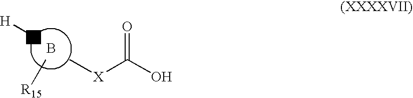

triflate (OTf), mesylate (OMs) or tosylate (OTs) to form compounds of the general formula (XXXXVI), using standard conditions or in the presence of a mixture of BuLi and diisopropylamine (to form LDA).

The preparation of compounds of the formula (III) comprises the below processes. (r1-r3)

r1) A compound of the formula LR^{c}R^{d} wherein L is a suitable leaving group, such as chloro, bromo, iodo could be transformed to the corresponding compound (III) using a sequence of reactions using first SMOPS* (*Baskin and Wang. Tetrahedron Letters, 2002, 43, 8479-83. See esp. page 8480, left hand column.) followed by hydrolysis using a base like NaOMe in an inert solvent like DMSO at room temperature. Followed by treatment by NH₂OSO₃H and NaOAc to give a compound of formula (III).

r2) A compound of the formula LSO₂R^{c}R^{d} wherein L is a suitable leaving group, such as chloro, bromo, iodo could be reacted with ammonium hydroxide in an inert solvent such as DCM to give a compound of formula (III).

r3) A compound of the formula LR^{c}R^{d} wherein L is a suitable leaving group, such as chloro, bromo, iodo could be transformed to the corresponding compound (III) using a sequence of reactions first Na₂SO₃, followed by a using a reagent such as PCl₅, POCl₃ or SOCl₂, followed by ammonium hydroxide to give a compound of formula (III).

At any stage in the synthesis of amine substituted pyridines, a halogen substituent in the 2, 4 or 6 position of the pyridine can be substituted with azide using known techniques. The azide can be reduced to the corresponding amine. These amines can subsequently be alkylated or acylated using known methods or with an alkylhalide or acylhalide, respectively.

Persons skilled in the art will appreciate that an acid can be transformed to the corresponding activated ester such as an acid chloride, followed by reaction with a thiol, R₁₆SH to give thioesters, R₁₆SC(O).

Persons skilled in the art will appreciate that an acid can be transformed to the corresponding activated ester such as an acid chloride, followed by reaction with a alcohol, R₆OH to give esters, R₆OC(O).

Persons skilled in the art will appreciate that a compound of formula (III) could be alkylated at the carbon atom in the alpha position to the sulfonamide using an alkylhalide. Preferably under basic conditions using a strong base such as sodium hydride.

Persons skilled in the art will appreciate that a nitrogen substituent at the 3 position of a pyridine could be replaced by a thioether chain, R₁₇S—, using known techniques or R₁₇SSR₁₇ and tert-Butylnitrite.

Persons skilled in the art will appreciate that a thioketone could be made from the corresponding ketone using known techniques or using Lawessons reagent.

Persons skilled in the art will appreciate that a pyridine N-oxide could be formed by from a pyridine using an oxidizing agent such as Urea hydrogen peroxide or hydrogen peroxide, with or without the presence of trifluoroaceticanhydrid.

The compounds of the invention may be isolated from their reaction mixtures using conventional techniques.

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)alkyl or benzyl esters. Suitable protecting groups for amino include allyl, t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned processes.

Persons skilled in the art will appreciate that, in order to obtain compounds of the invention in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

Persons skilled in the art will appreciate that starting materials for any of the above processes can in some cases be commercially available.

Persons skilled in the art will appreciate that processes could for some starting materials above be found in the general common knowledge.

The type of chemistry involved will dictate the need for protecting groups as well as sequence for accomplishing the synthesis. The use of protecting groups is fully described in “Protective groups in Organic

Chemistry”, edited by J W F McOmie, Plenum Press (1973), and “Protective Groups in Organic Synthesis”, 3^{rd} edition, T. W. Greene & P. G. M Wutz, Wiley-Interscience (1999).

Protected derivatives of the invention may be converted chemically to compounds of the invention using standard deprotection techniques (e.g. under alkaline or acidic conditions). The skilled person will also appreciate that certain compounds of formula (II)-(XXXXVII) and (L)-(LV) may also be referred to as being “protected derivatives”.

Compounds of the invention may also contain one or more asymmetric carbon atoms and may therefore exhibit optical and/or diastereoisomerism. Diastereoisomers may be separated using conventional techniques, e.g. chromatography or crystallization. The various stereisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerization, or by derivatisation, for example with a homochiral acid followed by separation of the diasteromeric derivatives by conventional means (e.g. HPLC, chromatography over silica or crystallization). Stereo centers may also be introduced by asymmetric synthesis, (e.g. metalloorganic reactions using chiral ligands). All stereoisomers are included within the scope of the invention. It will also be understood that some of the compounds described in the processes above may exhibit the phenomenon of tautomerism and the processes described above includes any tautomeric form.

All novel intermediates form a further aspect of the invention.

Salts of the compounds of formula (I) may be formed by reacting the free acid, or a salt thereof, or the free base, or a salt or a derivative thereof, with one or more equivalents of the appropriate base (for example ammonium hydroxide optionally substituted by C₁-C₆-alkyl or an alkali metal or alkaline earth metal hydroxide) or acid (for example a hydrohalic (especially HCl), sulphuric, oxalic or phosphoric acid). The reaction may be carried out in a solvent or medium in which the salt is insoluble or in a solvent in which the salt is soluble, e.g. water, ethanol, tetrahydrofuran or diethyl ether, which may be removed in vacuo, or by freeze drying. The reaction may also carried out on an ion exchange resin. The non-toxic physiologically acceptable salts are preferred, although other salts may be useful, e.g. in isolating or purifying the product.

### Pharmacological Data

Functional inhibition of—the P2Y₁₂ receptor can be measured by in vitro assays using cell membranes from P2Y₁₂ transfected CHO-cells, the methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation

y=A+((B−A)/(1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.

Most of the compounds of the invention have an activity, when tested in the functional inhibition of 2-Me-S-ADP induced P2Y₁₂ signalling assay described, at a concentration of around 2 μM or below.

For example the compounds described in Examples 7 and 35 gave the following test result in the functional inhibition of 2-Me-S-ADP induced P2Y₁₂ signalling assay described.

| | IC₅₀ (μM) |
|---|---|
| Example 7 | 0.13 |
| Example 35 | 0.09 |

The compounds of the invention act as P2Y₁₂ receptor antagonists and are therefore useful in therapy. Thus, according to a further aspect of the invention there is provided a compound of formula (I), or a pharmaceutically acceptable salt thereof, for use in therapy.

Thus, according to another further aspect of the invention there is provided a compound of formula (I), or a pharmaceutically acceptable salt thereof, for use as a medicament.

In a further aspect there is provided the use of a compound of formula (I), or a pharmaceutically acceptable salt thereof, for the manufacture of a medicament for treatment of a platelet aggregation disorder. In another aspect of the invention there is provided the use of a compound of formula (I), or a pharmaceutically acceptable salt thereof, for the manufacture of a medicament for the inhibition of the P2Y₁₂ receptor.

In yet another aspect of the invention there is provided a compound of formula (I), or a pharmaceutically acceptable salt thereof, for use as an inhibitor of the P2Y₁₂ receptor.

In still another aspect of the invention there is provided a compound of formula (I), or a pharmaceutically acceptable salt thereof, for use in the treatment of platelet aggregation disorder.

The compounds are useful in therapy, especially adjunctive therapy, particularly they are indicated for use as: inhibitors of platelet activation, aggregation and degranulation, promoters of platelet disaggregation, anti-thrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopaenic purpura, haemolytic uraemic syndrome, thrombotic complications of septicaemia, adult respiratory distress syndrome, anti- phospholipid syndrome, heparin-induced thrombocytopaenia and pre-eclampsia/eclampsia, or venous thrombosis such as deep vein thrombosis, venoocclusive disease, haematological conditions such as myeloproliferative disease, including thrombocythaemia, sickle cell disease; or in the prevention of mechanically-induced platelet activation in vivo, such as cardio-pulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of formula (I), or a pharmaceutically acceptable salt thereof, together with a pharmaceutically acceptable diluent, adjuvant and/or carrier.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler® in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol, mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be further illustrated with the following non-limiting examples:

### Examples

### General Experimental Procedure

Mass spectra was recorded on a Finnigan LCQ Duo ion trap mass spectrometer equipped with an electrospray interface (LC-MS) or LC-MS system consisting of a Waters ZQ using a LC-Agilent 1100 LC system.
¹H NMR measurements were performed on a Varian Mercury VXR 300 and 400 spectrometer, operating at a 1H frequency of 300 and 400 and Varian UNITY plus 400, 500 and 600 spectrometers, operating at 1H frequencies of 400, 500 and 600 respectively.

Chemical shifts are given in ppm with the solvent as internal standard. Protones on heteroatoms such as NH and OH protons are only reported when detected in NMR and can therefore be missing.
HPLC separations were performed on a Waters YMC-ODS AQS-3 120 Angstrom 3×500 mm or on a Waters Delta Prep Systems using Kromasil C₈, 10 μm columns.
Purification Method A: The purification system and LC-MS system used in purification Method A, referred to in some of the examples below, was Waters Fraction Lynx I Purification System: Column: Sunfire Prep C₁₈, 5 μm OBD, 19×150 mm column. Gradient 5-95% CH₃CN in 0.1 mM HCOOH (pH=3). MS triggered fraction collection was used. Mass spectra were recorded on either Micromass ZQ single quadropole or a Micromass quattro micro, both equipped with a pneumatically assisted electrospray interface.
Reactions performed in a microwave reactor were performed in a Personal Chemistry Smith Creator, Smith synthesizer or an Emrys Optimizer.
IUPAC names were generated with ACDLabs Name: Release 9:00, Product version 9.04.

The GTPγS values (IC50 in μM) mentioned in the examples below were measured using the method described below:

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation

y=A+((B−A)/(1+((C/x)AD)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.

### LIST OF USED ABBREVIATIONS

| Abbreviation | Explanation |
|---|---|
| AcOH | Acetic acid |
| aq | Aqueous |
| Boc | tert-butyloxycarbonyl |
| br | Broad |
| Brine | A saturated solution of sodium chloride in water |
| BSA | Bovine Serum Albumine |
| CDI | Carbonyldiimidazole |
| d | Doublet |
| DCM | Dichloromethane |
| DDQ | 2,3-Dichloro-5,6-dicyano-1,4-benzoquinone |
| DIPEA | N,N-Diisopropylethylamine |
| DMA | N,N-Dimethylacetamide |
| DMF | N,N-dimethylformamide |
| DMSO | Dimethylsulphoxide |
| EDCI | N-[3-(dimethylamino)propyl]-N′-ethylcarbodiimide |
| | hydrochloride |
| EtOAc | Ethyl acetate |
| EtOH | Ethanol |
| FA | Formic acid |
| HEPES | [4-(2-hydroxyethyl)-1-piperazineethanesulfonic |
| | acid |
| HFA | Hydrofluoroalkanes |
| HOAc | Acetic acid |
| HOBt | 1-Hydroxybenzotriazole |
| HPLC | High-performance liquid chromatography |
| Hz | Hertz |
| IPA | Isopropylalcohol |
| iPr | isopropyl |
| iPrOAc | isopropylacetate |
| J | Coupling constant |
| LC | Liquid chromatography |
| m | Multiplet |
| MeCN | Acetonitrile |
| MeOH | Methanol |
| MHz | Megahertz |
| mL | Millilitre |
| MS | Mass spectra |
| Ms | Methyl sulfonyl |
| MTBE | Methyl-tert-butylether |
| NCS | N-chlorosuccinimide |
| NMP | N-methylpyrrolidone |
| NMR | Nuclear magnetic resonance |
| OAc | acetate |
| Ph | Phenyl |
| PyBrop | Bromo(tripyrrolidin-1-yl)phosphonium |
| | hexafluorophosphate |
| q | Quartet |
| r.t | Room temperature |
| s | Singlet |
| t | triplet |
| TB | Tyrodes Buffer |
| TBTU | N-[(1H-1,2,3-benzotriazol-1- |
| | yloxy)(dimethylamino)methylene]-N- |
| | methylmethanaminium tetrafluoroborate |
| TEA | Triethylamine |
| Tf | Trifluoromethylsulfonyl |
| TFA | Trifluoroacetic acid |
| THF | Tetrahydrofurane |
| TMEDA | N,N,N′,N′-tetramethylethylendiamine |
| Ts | p-toluenesulfonyl |

### Sulfone Amides

### Synthesis of Sulfone Amides

The synthesis of the sulfonamides used in the examples below was made with one of the three methods described below:
i) By reacting the corresponding sulfonyl chloride with ammonia in THF or MeOH or by treatment with ammonium hydroxide in methylene chloride. The sulfonamides obtained was used without further purification.
ii) By essentially following the procedure described by Seto, T. et. al. in J. Organic Chemistry, Vol 68, No 10 (2003), pp. 4123-4125.
or
iii) By essentially following the procedure described by Wang, Z et. al. in Tetrahedron Letters, Vol 43 (2002), pp 8479-8483.

### Example 1

### Ethyl 6-{4-[(benzylsulfonyl)carbamoyl]piperidin-1-yl}-5-cyano-2-methoxynicotinate

### (a) tert-Butyl 4-{[(benzylsulfonyl)amino]carbonyl}piperidine-1-carboxylate

TEA (591 g, 5840 mmol) was added to a stirred suspension of 1-(tert-butoxycarbonyl)piperidine-4-carboxylic acid (448 g, 1954 mmol), LiCl (23.1 g, 545 mmol) and TBTU (657 g, 2046 mmol) in THF (3000 mL) under an atmosphere of nitrogen at r.t. A solution of 1-phenylmethanesulfonamide (352 g in 1300 mL THF, 2056 mmol) was added after 1.5 hours and the stirring was continued over night. The solvent was removed in vaccuo to give a thick grey-beige slurry (volume about 2500 mL). EtOAc (3500 mL) was added followed by an aqueous solution of HCl (1960 mL 3.6 M HCl and 1960 mL water). The water phase was removed and the organic phase was washed with 2×1500 mL 1 M HCl. The organic phase was cooled to 0° C. which gave a precipitate of HOBt that was filtered off. Most of the solvent was removed in vaccuo to give a thick grey-white slurry. EtOH (50%, 4000 mL) was added and the slurry was stirred for 1.5 hours. The precipitated product was filtered off, washed with 50% EtOH (500 mL+2×1500 mL) and dried in a vaccum oven at 25° C. to give tert-butyl 4-[(benzylsulfonyl)carbamoyl]piperidine-1-carboxylate as a white solid. Yield 584 g (78%).

¹H NMR (400 MHz, CDCl₃): δ 1.46 (9H, s), 1.54-1.61 (2H, m), 1.70-1.74 (2H, m), 2.19-2.27 (1H, m), 2.68-2.75 (2H, m), 4.07-4.12 (2H, m), 4.66 (2H, s), 7.32-7.41 (5H, m), 7.54 (1H, br s).

### (b) tert-Butyl 4-[allyl(benzylsulfonyl)carbamoyl]piperidine-1-carboxylate

A mixture of tert-butyl 4-[(benzylsulfonyl)carbamoyl]piperidine-1-carboxylate (11.47 g, 30 mmol), 3-bromoprop-1-ene (10.89 g, 90 mmol) and DIPEA (7.76 g, 60 mmol) in DMF (30 mL) was stirred at r.t. for 21 hours. Water (75 mL) was added and the aqueous phase was extracted with heptane/DCM 4/1 (3×75 mL). The combined organic phase was dried (MgSO₄), filtered and evaporated to give the product which was used without further purification.

### (c) N-allyl-N-(benzylsulfonyl)piperidine-4-carboxamide trifluoroacetate

TFA/DCM 2/1 (30 mL) was added to a stirred solution of tert-butyl 4-[allyl(benzylsulfonyl)carbamoyl]piperidine-1-carboxylate (12.676 g, 30 mmol) in DCM (10 mL) at 0° C. (ice/water bath) and the stirring was continued for 5 minutes followed by 4 hours at r.t. The solvent was evaporated and the mixture was co-evaporated with DCM is twice to give the product as a TFA salt which was used in the next step without further purification.

### (d) N-allyl-N-(benzylsulfonyl)-1-(2-cyanoethanimidoyl)piperidine-4-carboxamide

N-allyl-N-(benzylsulfonyl)piperidine-4-carboxamide trifluoroacetate (30 mmol) was added to a cold (ice/water bath temperature) solution of ethyl 2-cyanoethanimidoate (See McElvain, S. M.; Schroeder, J. P.; J. Am. Chem. Soc. 71, p. 40(1949)) (15.14 g, 101.25 mmol, 75% pure) and DIPEA (23.26 g, 180 mmol) in EtOH (200 mL) and the mixture was stirred for 10 minutes followed by 16 hours at r.t. LC-MS showed complete conversion of the starting material. This solution was used in the next step as such.

### (e) Ethyl 6-{4-[allyl(benzylsulfonyl)carbamoyl]piperidin-1-yl}-5-cyano-2-oxo-1,2-dihydropyridine-3-carboxylate

Diethyl(ethoxymethylene)malonate (8.43 g, 39 mmol) was added to the solution from step (d) above and the reaction mixture was stirred for 18 hours at r.t. Evaporation of the solvent gave 32 g of a crude product. 8 g (¼) of this was taken out and purified by preparative HPLC (Kromasil C₈ 10 μm, Eluent: A: CH₃CN; B: 0.2% HOAc in water/CH₃CN 95/5; C: 0.1 M NH₄OAc/CH₃CN 95/5. Using A/B/C 5/0/95 during injection and then eluting with a gradient going from A/B/C 5/95/0 to 100/0/0) to give two fractions containing the product. Fraction 1: 308 mg (8% chemical yield, 100% purity according to LC-MS and Fraction 2: 853 mg (76% pure according to LC-MS).

¹H-NMR(400 MHz, CDCl₃): δ 1.40 (3H, t, J=7.2 Hz), 1.57-1.80 (4H, m), 2.60-2.70 (1H, m), 2.92-3.03 (2H, m), 4.11-4.16 (2H, m), 4.39 /2H, q, J=7.2 Hz), 4.61 (2H, s), 4.64-4.72 (2H, m), 5.19-5.30 (2H, m), 6.62-5.75 (1H, m), 7.31-7.45 (5H, m), 8.24 (1H, s), 11.90 (1H, br. s, NH).

### (f) Ethyl 6-{4-[allyl(benzylsulfonyl)carbamoyl]piperidin-1-yl}-5-cyano-2-methoxynicotinate

Silver carbonate (23 mg, 0.084 mmol) and methyl iodide (85 mg, 0.6 mmol) was added to a solution of ethyl 6-{4-[allyl(benzylsulfonyl)carbamoyl]piperidin-1-yl}-5-cyano-2-oxo-1,2-dihydropyridine-3-carboxylate (31 mg, 0.06 mmol) in DCM (0.6 mL) in a Smith process vial. The vial was sealed and wrapped in aluminum foil and stirred at r.t. for 21 hours. LC/MS showed no right mass. 1 mL DMSO was added and the vial heated to 100° C. for 10 minutes in a microwave oven, single node heating. LC/MS showed full conversion. The material was filtered and evaporated and 10 mL NaHCO₃ (sat) was added and the mixture was extracted with 3×10 mL EtOAc. The organic phases were combined and extracted with brine, dried (Na₂SO₄), filtered and concentrated to give ethyl 6-{4-[allyl(benzylsulfonyl)carbamoyl]piperidin-1-yl}-5-cyano-2-methoxynicotinate which was used without further purification. Yield: 21 mg

### (g) Ethyl 6-{4-[(benzylsulfonyl)carbamoyl]piperidin-1-yl}-5-cyano-2-methoxynicotinate

Sodium 4-methylbenzenesulfinate (222 mg, 1.24 mmol) and Pd(PPh₃)₄ (67 mg, 0.058 mmol) was added to a solution of ethyl 6-{4-[allyl(benzylsulfonyl)carbamoyl]piperidin-1-yl}-5-cyano-2-methoxynicotinate (437 mg, 0.830 mmol) under an atmosphere of nitrogen and the mixture was stirred for 2 hours at r.t. The solvent was removed in vacuo and the residue was purified by preparative HPLC (Kromasil C₈ 10 μm, Eluent: A: CH₃CN; B: 0.2% HOAc in water/CH₃CN 95/5; C: 0.1 M NH₄OAc/CH₃CN 95/5. Using A/B/C 5/0/95 during injection and then eluting with a gradient going from A/B/C 5/95/0 to 100/0/0) to give the desired product. Yield: 129 mg (34%).

¹H NMR (300 MHz, CDCl₃): δ 1.36 (3H, t, J=7.2 Hz), 1.76-1.85 (2H, m), 1.85-1.93 (2H, m), 2.40-2.48 (1H, m), 3.13-3.22 (2H, m), 4.00 (3H, s), 4.30 (2H, q, J=7.2 Hz), 4.61-4.67 (4H, m), 7.31-7.36 (2H, m), 7.37-7.43 (3H, m), 8.33 (1H, s).

MS m/z: 487 (M+1)

GTPγS(IC_{50 1} μM): 0.012

### Example 2

### Ethyl 6-{3-[(benzylsulfonyl)carbamoyl]azetidin-1-yl}-5-cyano-2-methoxynicotinate

### (a) 1-(Trifluoroacetyl)azetidine-3-carboxylic acid

Trifluoroacetic anhydride (93.5 g, 445 mmol) was added to solid acetidine-3-carboxylic acid (15 g, 148 mmol) at 0° C. (ice/water bath cooling). The mixture was stirred manually with a spatula for 30 minutes followed by mechanical stirring (the mixture became homogenous after 40 minutes) for another 2 hours and 40 minutes. The mixture was concentrated in vacuo and the residual yellow oil was partitioned between EtOAc (300 mL) and water (50 mL). The phases was separated and the organic phase was washed with water (2×50 mL) and Brine (20 mL), dried (Na₂SO₄), filtered and evaporated to give a yellow oil. Drying in vacuo at r.t. over night gave the product as a yellow solid. Yield: 29.2 g (100 %).

### (b) tert-Butyl 1-(trifluoroacetyl)azetidine-3-carboxylate

1,1-di-tert-butoxy-N,N-dimethylmethanamine (16.5 g, 81 mmol) was added to a solution of 1-(trifluoroacetyl)azetidine-3-carboxylic acid (5 g, 25 mmol) and the mixture was heated to reflux for 8 hours. LC-MS showed remaining starting material and therefore an additional amount of 1,1-di-tert-butoxy-N,N-dimethylmethanamine (21.2 g, 81 mmol) was added and the heating was continued over night. LC-MS showed still some remaining starting material (starting material/product about 1/2) and the THF was exchanged for toluene (100 mL) and the mixture heated to 100° C. (oil bath temperature) for 2 hours. The solvent was evaporated and the residue dissolved in EtOAc (200 mL). The organic phase was washed with NaHCO₃(sat) (2×50 mL), water (2×50 mL), Brine (50 mL), dried (Na₂SO₄), filtered and evaporated to give the desired product. Yield: 4.5 g (70%).

### (c) tert-Butyl azetidine-3-carboxylate

Potassium carbonate (7.37 g, 53.3 mmol) was added to a solution of tert-butyl 1-(trifluoroacetyl)azetidine-3-carboxylate (4.5 g, 17.8 mmol) in methanol/water (7/3, 71 mL) and the mixture was stirred at r.t for 3.5 hours. The methanol was evaporated and DCM (200 mL) was added. The phases were separated and the water phase was extracted with DCM (2×100 mL) The combined organic phase was washed with water (2×50 mL), brine (1×50 mL), dried (Na₂SO₄), filtered and evaporated to give the desired product as a yellow oil. Yield: 1.19 g (40%).

### (d) tert-butyl 1-(2-cyanoethanimidoyl)azetidine-3-carboxylate

A microwave vial was charged with tert-butyl azetidine-3-carboxylate (1.1 g, 6.65 mmol, 95% pure), ethyl 2-cyanoethanimidoate (See McElvain, S. M.; Schroeder, J. P.; J. Am. Chem. Soc. 71, p. 40(1949)) (1.12 g, 7.98 mmol, 80% pure) and EtOH (15 mL) and heated to 100° C. for 10 minutes. This mixture was used as such in the next step assuming 100% yield.

### (e) Ethyl 6-[3-(tert-butoxycarbonyl)azetidin-1-yl]-5-cyano-2-oxo-1,2-dihydropyridine-3-carboxylate

Diethyl(ethoxymethylene)malonate (2.16 g, 9.98 mmol) was added to the solution from step (d) above and the reaction mixture was stirred at r.t for 18 hours followed by 10 minutes at 100° C. and 10 minutes at 110° C. using microwave single node heating. The solvent was evaporated and the residue was dissolved in DCM and passed through a plug of silica gel (Eluted with DCM (100%), DCM/MeOH (10/1), (5/1) and (1/1). The fractions containing the product was collected and evaporated to give a crude product (3.1 g). The crude product was purified by preparative HPLC (Kromasil C₈, 10 μm, using a gradient of 25 to 70% CH₃CN/0.2% HOAc in water) to give the desired product after freeze drying. Yield: 1.043 g (36%).

### (f) Ethyl 6-[3-(tert-butoxycarbonyl)azetidin-1-yl]-5-cyano-2-methoxynicotinate

A microwave vial was charged with ethyl 6-[3-(tert-butoxycarbonyl)azetidin-1-yl]-5-cyano-2-oxo-1,2-dihydropyridine-3-carboxylate (915 mg, 2.11 mmol), methyl iodide (2.99 g, 21.1 mmol), silver carbonate (1.74 g, 6.32 mmol), DMSO (10 mL) and heated to 80° C. for 2+2 minutes. Addition of DCM and filtration of the precipitated solids (washed the filtercake with DCM) followed by evaporation of the DCM and purification of the crude product by preparative HPLC (Kromasil C₈, 10 μm, using a gradient of 30 to 100% CH₃CN/0.1 M NH₄OAc) to give the desired product after freeze drying. Yield: 565 mg (74%).

### (g) 1-[3-cyano-5-(ethoxycarbonyl)-6-methoxypyridin-2-yl]azetidine-3-carboxylic acid

TFA (4.63 mL, 62.3 mmol) was added to a solution of ethyl 6-[3-(tert-butoxycarbonyl)azetidin-1-yl]-5-cyano-2-methoxynicotinate (563 mg, 1.56 mmol) in DCM (15 mL) and the mixture was stirred at r.t for 4 hours. The solvent and excess TFA was removed and the residue dried in vacuo over night to give the desired crude product which was used in the next step without further purification. Yield: 493 mg (104%,).

1H NMR (400 MHz, DMSO-d₆) δ 1.24 (31I, t, J=7.05 Hz), 3.51-3.60 (1H, m), 3.89 (3H, s), 4.17 (2H, q, J=7.05 Hz), 4.30-4.40 (2H, m), 4.45-4.55 (2H, m), 8.22 (1H, m).

### (h) Ethyl 6-{3-[(benzylsulfonyl)carbamoyl]azetidin-1-yl}-5-cyano-2-methoxynicotinate

PyBrop (45.2 mg, 0.097 mmol) was added to a solution of 1-[3-cyano-5-(ethoxycarbonyl)-6-methoxypyridin-2-yl]azetidine-3-carboxylic acid (14.8 mg, 0.048 mmol) and DIPEA (62.7 mg, 0.485 mmol) in DCM (2 mL) and the reaction was stirred at r.t for 2 hours. The solvent was removed and the crude product was purified by preparative HPLC (Kromasil C₈, 10 μm, using a gradient of 30 to 100% CH₃CN/0.1 M NH₄OAc) to give the desired product. Yield: 12 mg (54%).

¹H NMR (400 MHz, DMSO-d₆): δ 1.25 (3H, t, J=7.05Hz), 3.50-3.60 (1H, m), 3.91 (3H, s), 4.18 (2H, q, J=7.05 Hz), 4.25-4.48 (4H, m), 4.73 (2H, s), 7.30-7.40 (5H, m), 8.24 (1H, s), 11.80 (1H, br s, NH)

MS m/z: 459 (M+1), 457 (M−1).

GTPγS(IC₅₀ μM): 0.018

### Example 3

### Ethyl 6-{4-[(benzylsulfonyl)carbamoyl]piperidin-1-yl}-5-cyano-2-ethoxynicotinate

### (a) Ethyl 6-{4-[allyl(benzylsulfonyl)carbamoyl]piperidin-1-yl}-5-cyano-2-ethoxynicotinate

Ethyl iodide (127.8 mg, 0.819 mmol) was added to a mixture of ethyl 6-{4-[allyl(benzylsulfonyl)carbamoyl]piperidin-1-yl}-5-cyano-2-oxo-1,2-dihydropyridine-3-carboxylate (100 mg, 0.164 mmol and silver carbonate (135.6 mg, 0.492 mmol) in CH₃CN (20 mL) and the mixture was heated to reflux for 3 hours. The mixture was filtered and concentrated to give'a crude product which was used in the next step without further purification.

### (b) Ethyl 6-{4-[(benzylsulfonyl)carbamoyl]piperidin-1-yl}-5-cyano-2-ethoxynicotinate

Sodium 4-methylbenzenesulfinate (79.2 mg, 0.444 mmol) and Pd(PPh₃)₄ (190 mg, 0.165 mmol) was added to a solution of ethyl 6-{4-{allyl(benzylsulfonyl)carbamoyl]piperidin-1-yl}-5-cyano-2-ethoxynicotinate (100 mg, 0.165 mmol) under an atmosphere of nitrogen and the mixture was stirred for 15 minutes at r.t. The solvent was removed in vacuo and the crude product was purified by preparative HPLC (Kromasil C₈, 10 μm, 50.8×300 mm column using a gradient of 30 to 100% CH₃CN/0.2% acetic acid in water) to give the desired product. Yield: 54 mg (65%).

¹H NMR (400 MHz, DMSO-d₆) δ 1.26 (3H, t, J=7.1 Hz), 1.33 (3H, t, J=7.3 Hz), 1.57-1.69 (2H, m), 1.78-1.86 (2H, m), 2.54-2.63 (1H, m), 3.11-3.21 (2H, m), 4.18 (2H, q, J=7.2 Hz), 4.38 (2H, q, J=7.2 Hz), 4.47-4.55 (2H, m), 4.68 (2H, s), 7.22-7.32 (2H, m), 7.33-7.43 (3H, m), 8.26 (1H, s), 11.59 (1H, s)

MS m/z: 501 (M+1)

GTPγS(IC₅₀ μM): 0.012

### Example 4

### Ethyl 6-{4-[(benzylsulfonyl)carbamoyl]piperidin-1-yl}-5-cyano-2-(ethylthio)nicotinate

### (a) Ethyl 6-{4-[allyl(benzylsulfonyl)carbamoyl]piperidin-1-yl}-5-cyano-2-{[(trifluoromethyl)sulfonyl]oxy}nicotinate

Trifluoromethanesulfonic anhydride (186 mg, 0.66 mmol) was added dropwise to a cold (ice/water bath temperature) solution of ethyl 6-{4-[allyl(benzylsulfonyl)carbamoyl]piperidin-1-yl}-5-cyano-2-oxo-1,2-dihydropyridine-3-carboxylate (308 mg, 0.6 mmol) and TEA (273 mg, 2.7 mmol) in DCM (7 mL). The reaction was stirred at 0° C. for 1 hour and NaHCO₃ (aq,sat) was added. The aqueous phase was extracted with DCM (2×10 mL). The combined organic phase was dried (Na₂SO₄), filtered and evaporated to give the product which was used without further purification.

### (b) Ethyl 6-{4-[(benzylsulfonyl)carbamoyl]piperidin-1-yl}-5-cyano-2-(ethylthio)nicotinate

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (116 mg, 0.18 mmol), Pd₂(dba)₃ (23 mg, 0.025 mmol), Xantphos(24 mg, 0.041 mmol), ethanthiol (0.1 mL, 1.35 mmol), DIPEA (0.1 mL, 0.57mmol) and dioxane(3mL) and the reaction mixture was heated to 160° C. for 5 minutes using microwave single node heating. LCMS showed two products (allyl-protected and allyl-deprotected product). NH₄Cl(aq) was added and the mixture was extracted with DCM (3 times). The combined organic layer was run through a phase separator and evaporated. The crude was purified by preparative HPLC (Kromasil C₈, 10 μm, 21.5×250 mm column, flow 25 mL/minute using a gradient of 40 to 80% CH₃CN/0.1 M NH₄OAc) to give the desired compound. Yield: 11 mg (12%).

¹H NMR (500 MHz, DMSO-d₆): δ 1.30 (6H, t, J=7.1 Hz), 1.61-1.71 (2H, m), 1.81-1.87 (2H, m), 2.57-2.65 (1H, m), 3.07 (2H, q, J=7.2 Hz), 3.18-3.25 (2H, m), 4.24 (2H, q, J=7.1 Hz), 4.52-4.57 (2H, m), 4.68 (2H, s), 7.28-7.31 (2H, m), 7.38-7.41 (3H, m), 8.28 (1H, s), 11.61 (1H, s).

MS m/z: 517 (M+1), 515 (M−1).

GTPγS(IC₅₀ μM): 0.006

### Example 5

### Ethyl 6-{4-[(benzylsulfonyl)carbamoyl]piperidin-1-yl}-2,5-dicyanonicotinate

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (113 mg, 0.17 mmol), Pd₂(dba)₃ (25 mg, 0.027 mmol), Xantphos(15 mg, 0.026 mmol), NaCN (29 mg, 0.59 mmol), DIPEA (0.1 mL, 0.57 mmol) and dioxane (5mL) and the reaction mixture was heated to 160° C. for 10minutes using microwave single node heating. The mixture was is filtered through a plug of Celite and washed with dioxane. Diethyl ether was added and the mixture was extracted with NaHCO₃(aq) (3 times). To the combined aqueous layer was added conc HCl until pH2 and the mixture was extracted with DCM(3 times). The combined organic layer was run through a phase separator and evaporated. The crude was purified by preparative HPLC (Kromasil C₈, 10 μm, 21.5×250 mm column, flow 25 mL/minute using a gradient of 10 to 40% CH₃CN/0.1 M NH₄OAc) to give the desired compound. Yield 19 mg (23%).

¹H NMR (500 MHz, DMSO-d₆): δ 1.34 (3H, t, J=7.1 Hz), 1.64-1.73 (2H, m), 1.85-1.91 (2H, m), 2.58-2.65 (1H, m), 3.21-3.28 (2H, m), 4.34 (2H, q, J=7.1 Hz), 4.47-4.52 (2H, m), 4.69 (2H, s), 7.28-7.32 (2H, m), 7.38-7.43 (3H, m), 8.59 (1H, s), 11.63 (1H, br s).

MS m/z: 482 (M+1), 480 (M−1).

GTPγS(IC₅₀ μM): 0.009

### Example 6

### Ethyl 6-{4-[(benzylsulfonyl)carbamoyl]piperidin-1-yl}-5-cyano-2-(hydroxymethyl)nicotinate

### (a) Ethyl 4-[(3,4-dimethoxybenzyl)oxy]-3-oxobutanoate

Prepared essentially according to the procedure described by Yasohara Y et al, (Tetrahedron assymetry, 12(2001) pp. 1713-18) replacing bensylalcohol for (3,4-dimethoxyphenyl)methanol. Yield: 9.65 g (44%).

¹H NMR (500 MHz, DMSO-d₆) δ 1.17 (3H, t, J=7.3 Hz), 3.57 (2H, s), 3.75 (3H, s), 3.76 (3H, s), 4.08 (2H, q, J=7.2 Hz), 4.20 (2H, s), 4.44 (2H, s), 6.84-6.96 (3H, m)

MS m/z: 295 (M−1)

### (b) Ethyl 5-cyano-2-{[(3,4-dimethoxybenzyl)oxy]methyl}-6-oxo-1,6-dihydropyridine-3-carboxylate

Prepared essentially by the same procedure as described in Example 35 (a) from Ethyl 4-[(3,4-dimethoxybenzyl)oxy]-3-oxobutanoate. Yield 3.53 g (56%).

¹H NMR (500 MHz, DMSO-d₆) δ 1.26 (3H, t, J=7.1 Hz), 3.74 (6H, d, J=3.1 Hz), 4.20 (2H, q, J=7.1 Hz), 4.53 (2H, s), 4.80 (2H, s), 6.86-7.00 (3H, m), 8.42 (1H, s)

MS m/z: 390 (M+NH₄), 371.3 (M−1)

### (c) Ethyl 5-cyano-2-{[(3,4-dimethoxybenzyl)oxy]methyl}-6-[(methylsulfonyl)oxy]nicotinate

DIPEA (260 mg, 2.01 mmol) and mesylchloride (81 mg, 2.01 mmol dissolved in DCM 2 mL) were added to a solution of ethyl 5-cyano-2-{[(3,4-dimethoxybenzyl)oxy]methyl}-6-oxo-1,6-dihydropyridine-3-carboxylate (250 mg, 0.671 mmol) and the reaction was stirred at r.t for about 10 minutes. This solution was used as such in step (e) below.

### (d) N-(benzylsulfonyl)piperidine-4-carboxamide

tert-Butyl 4-[(benzylsulfonyl)carbamoyl]piperidine-1-carboxylate (See Example 1(a)) (583 g, 1524 mmol) was suspended in formic acid (3000 mL) under a nitrogen atmosphere and the reaction was stirred for 20 minutes. The reaction was foaming due to the gas evolution and formic acid (500 mL) was used to wash down the foam from the reaction vessel walls.

After 2 hours the foaming had stopped and the reaction was clear with a few solids left. The reaction was stirred over night and 2500 ml of formic acid was removed in vaccuo. Water (1000 mL) was added and the reaction was filtered. The clear solution was evaporated and water (3000 mL) was added. A saturated ammonium hydroxide solution in water was used (totally 390 mL was added and the pH was going from 3.10 to 6.10) to neutralize the acidic solution and at the endpoint (pH=6.10) a heavy precipitate of the product was formed. The mixture was stirred over night and the precipitate was filtered off and washed with water (1000 mL). Drying in a vaccum oven at 25° C. gave N-(benzylsulfonyl)piperidine-4-carboxamide as a white powder. Yield 372.4 g (87%).

¹H NMR (400 MHz, DMSO-d₆): δ 1.60-1.72 (2H, m), 1.75-1.84 (2H, m), 2.10-2.19 (1H, m), 2.77-2.87 (2H, m), 3.10-3.18 (2H, m), 4.23 (2H, s), 7.18-7.28 (5H, m), 8.17 (1H, br s).

### (e) Ethyl 6-{4-[(benzylsulfonyl)carbamoyl]piperidin-1-yl}-5-cyano-2-{[(3,4-dimethoxybenzyl)oxy]methyl}nicotinate

N-(benzylsulfonyl)piperidine-4-carboxamide (208 mg, 0.737 mmol) was added to the reaction mixture from step (c) above and the mixture was heated to 100° C. in a microwave oven for a total time of 25 minutes. Water was added and the aquoeus phase was acidified with 1 M HCl (0.7 mL). The organic phase was evaporated and the crude product was purified by preparative HPLC (Kromasil C₈, 10 μm, using a gradient of 5-50% CH3CN/0.1 M NH4OAc (pH 5)) to give the desired compound. Yield: 87 mg (20%).

¹H NMR (500 MHz, THF-d₈) δ 1.35 (3H, t, J=7.2 Hz), 1.80-1.88 (4H, m), 2.43-2.50 (1H, m), 3.17-3.25 (2H, m), 3.77 (3H, s), 3.79 (3H, s), 4.30 (2H, q, J=7.1 Hz), 4.595 (2H, s), 4.63 (2H, s), 4.68-4.75 (4H, m), 4.89 (2H, s), 6.84 (2H, s), 6.95 (1H, s), 7.31-7.40 (5H, m), 8.34 (1H, s)

MS m/z: 637 (M+1), 635 (M−1)

### (e) Ethyl 6-{4-[(benzylsulfonyl)carbamoyl]piperidin-1-yl}-5-cyano-2-(hydroxymethyl)nicotinate

DDQ (31 mg, 0.137 mmol) was added to a solution of ethyl 6-{4-[(benzylsulfonyl)carbamoyl]piperidin-1-yl}-5-cyano-2-{[(3,4-dimethoxybenzyl)oxy]methyl}nicotinate (87 mg, 0.137 mmol) in DCM (1 mL) and water (1 mL) was added to give a bi-phasic mixture which was stirred at r.t for 60 minutes to give a clean conversion to the product. The crude product could be purified by preparative HPLC.

MS m/z: 637

GTPγS(IC₅₀ μM): 0.017

### Example 7

### Ethyl 5-cyano-2-methoxy-6-{4-[(phenylsulfonyl)carbamoyl]piperidin-1-yl}nicotinate

### (a) tert-Butyl 1-(2-cyanoethanimidoyl)piperidine-4-carboxylate

Two microwave vials was each charged with ethyl 2-cyanoethanimidoate (See McElvain, S. M.; Schroeder, J. P.; J. Am. Chem. Soc. 71, p. 40(1949)) (841 mg, 7.7 mmol), tert-butyl piperidine-4-carboxylate (926 mg, 5 mmol), DIPEA (1.94 g, 15 mmol), EtOH (7.5 mL) and heated to 100° C. for 10 minutes in a microwave oven, single node heating. Additional ethyl 2-cyanoethanimidoate (252 mg, 4.5 mmol) and DIPEA (969 mg, 7.5 mmol) was added to each vial and the stirring was continued at r.t for 16 hours. LC-MS showed still some remaining tert-butyl piperidine-4-carboxylate and therefore ethyl 2-cyanoethanimidoate (246 mg, 2.2 mmol) was added and the mixture was again heated to 100° C. in a microwave oven for 20 minutes. The solutions from the vials was combined and used without further purification in the next step.

### (b) Ethyl 6-[4-(tert-butoxycarbonyl)piperidin-1-yl]-5-cyano-2-oxo-1,2-dihydropyridine-3-carboxylate

Diethyl(ethoxymethylene)malonate (3.24 g, 15 mmol) was added to the solution from step (a) above and the reaction mixture was stirred at r.t for 16 hours. The solvent was evaporated and the NaHCO₃(sat) (50 mL) was added and the water phase extracted with DCM (3×50 mL). The combined organic phase was washed with brine (150 mL), dried (Na₂SO₄), filtered and evaporated to give a crude product which was purified by preparative HPLC (Kromasil C₈, 10 μm, 50.8×250 mm column, flow 50 mL/minute using a gradient of 5 to 100% CH₃CN/0.1 M NH₄OOCH) to give the desired product. Yield: 1.262 g (32%).

¹H NMR (500 MHz, CDCl₃): δ 1.41 (3H, t, J=7.1 Hz), 1.46 (9H, s), 1.75-1.86 (2H, m), 1.98-2.06 (2H, m), 2.53-2.61 (1H, m), 3.29-3.37 (2H, m), 4.39 (2H, q, J=7.1 Hz), 4.53-4.61 (2H, m), 8.20 (1H, s). Not unambiguous where NH proton is.

MS m/z: 376 (M+1)

### (c) Ethyl 6-[4-(tert-butoxycarbonyl)piperidin-1-yl]-5-cyano-2-methoxynicotinate

A microwave vial was charged with ethyl 6-[4-(tert-butoxycarbonyl)piperidin-1-yl]-5-cyano-2-oxo-1,2-dihydropyridine-3-carboxylate (188 mg, 0.5 mmol), methyl iodide (355 mg, 2.5 mmol), silver carbonate (276 mg, 1 mmol), DMSO (2.5 mL) and heated to 100° C. a microwave oven, single node heating, for 20 minutes. LC-MS showed 81% of O-alkylated product along with 19% N-alkylated product. The crude product was purified by preparative HPLC (Kromasil C₈ 10 μm, Eluent: A: CH₃CN; B: 0.2% HOAc in water/CH₃CN 95/5; C: 0.1 M NH₄OAc/CH₃CN 95/5. Using A/B/C 5/0/95 during injection and then eluting with a gradient going from A/B/C 5/95/0 to 100/0/0) to give the desired product. Yield: 141 mg (72%).

¹H NMR (400 MHz, CDCl₃): δ 1.35 (3H, t, J=7.2 Hz), 1.46 (9H, s), 1.75-1.86 (2H, m), 1.97-2.06 (2H, m), 2.51-2.60 (1H, m), 3.27-3.37 (2H, m), 3.99 (3H, s), 4.30 (2H, q, J=7.2 Hz), 4.51-4.60 (2H, m), 8.32 (1H, s).

MS m/z: 390 (M+1)

### (d) 1-[3-Cyano-5-(ethoxycarbonyl)-6-methoxypyridin-2-yl]piperidine-4-carboxylic acid

TFA/DCM 1/1 (10 mL) was added to ethyl 6-[4-(tert-butoxycarbonyl)piperidin-1-yl]-5-cyano-2-methoxynicotinate (476 mg, 1.22 mmol) and the solution was stirred for 2 hours at r.t. The solvent was evaporated and the residue was co-evaporated with DCM twice to give a crude product which was used without further purification. Yield: 435 mg (107%).

¹H NMR (400 MHz, CDCl₃): δ 1.34 (3H, t, J=7.1 Hz), 1.80-1.93 (2H, m), 2.04-2.13 (2H, m), 2.66-2.76 (1H, m), 3.29-3.39 (2H, m), 3.97 (3H, s), 4.26-4.34 (2H, q, J=7.1 Hz), 4.52-4.61 (2H, m), 8.32 (1H, s), 9.94 (1H, br s).

MS m/z 334 (M+1)

### (e) Ethyl 5-cyano-2-methoxy-6-{4-[(phenylsulfonyl)carbamoyl]piperidin-1-yl}nicotinate

DIPEA (129.2 mg, 1 mmol) was added after 1 minute to a solution of 1-[3-Cyano-5-(ethoxycarbonyl)-6-methoxypyridin-2-yl]piperidine-4-carboxylic acid (33.3 mg, 0.1 mmol), benzenesulfonamide (18 mg, 0.115 mmol) and PyBrop (70 mg, 0.15 mmol) in DCM (2 mL) and the mixture was stirred at r.t for 16 hours.

The solvent was evaporated and the crude product purified according the purification Method A (See General Experimental Procedure) to give the desired product. Yield: 2 mg (4%).

MS m/z: 473 (M+1)

GTPγS(IC₅₀ μM): 0.134

### Example 8

### Ethyl 5-cyano-6-(4-{[(2-fluorobenzyl)sulfonyl]carbamoyl}piperidin-1-yl)-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(2-fluorophenyl)methanesulfonamide (22 mg, 0.115 mmol). Yield: 13.6 mg (27%).

¹H NMR (600 MHz, DMSO-d₆): δ 1.26 (3H, t, J=7.2 Hz), 1.62-1.70 (2H, m), 1.85-1.91 (2H, m), 2.40-2.48 (1H, m, hidden under DMSO signal), 3.16-3.23 (2H, m), 3.92 (3H, s), 4.20 (2H, q, J=7.2 Hz), 4.53-4.59 (2H, m), 4.75 (2H, s), 7.23-7.29 (2H, m), 7.38-7.43 (1H, m), 7.43-7.48 (1H, m), 8.28 (1H, s).

MS m/z: 505 (M+1)

GTPγS(IC₅₀ μM): 0.01

### Example 9

### Ethyl 6-(4-{[(2-chlorobenzyl)sulfonyl]carbamoyl}piperidin-1-yl)-5-cyano-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(2-chlorophenyl)methanesulfonamide (25 mg, 0.115 mmol). Yield: 17.2 mg (33%).

¹H NMR (600 MHz, DMSO-d₆): δ 1.26 (3H, t, J=7.2 Hz), 1.63-1.71 (2H, m), 1.87-1.93 (2H, m), 2.40-2.48 (1H, m, hidden under DMSO signal), 3.16-3.23 (2H, m), 3.92 (3H, s), 4.20 (2H, q, J=7.2 Hz), 4.53-4.59 (2H, m), 4.86 (2H, s), 7.39-7.47 (3H, m), 7.52-7.54 (1H, m), 8.28 (1H, s).

MS m/z: 521 (M+1)

GTPγS(IC₅₀ μM): 0.032

### Example 10

### Ethyl 5-cyano-6-(4-{[(3-fluorobenzyl)sulfonyl]carbamoyl}piperidin-1-yl)-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(3-fluorophenyl)methanesulfonamide (22 mg, 0.115 mmol). Yield: 16.3 mg (32%).

¹H NMR (600 MHz, DMSO-d₆): δ 1.26 (3H, t, J=7.2 Hz), 1.60-1.69 (2H, m), 1.80-1.86 (2H, m), 2.40-2.48 (1H, m, hidden under DMSO signal), 3.15-3.22 (2H, m), 3.92 (3H, s), 4.20 (2H, q, J=7.2 Hz), 4.52-4.58 (2H, m), 4.74 (2H, s), 7.11-7.15 (2H, m), 7.22-7.27 (1H, m), 7-43-7.49 (1H, m), 8.28 (1H, s).

MS m/z: 505 (M+1)

GTPγS(IC₅₀ μM): 0.016

### Example 11

### Ethyl 5-cyano-6-(4-{[(4-fluorobenzyl)sulfonyl]carbamoyl}piperidin-1-yl)-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(4-fluorophenyl)methanesulfonamide (22 mg, 0.115 mmol). Yield: 20.4 mg (40%).

¹H NMR (600 MHz, DMSO-d₆): δ 1.26 (3H, t, J=7.2 Hz), 1.60-1.68 (2H, m), 1.82-1.87 (2H, m), 2.40-2.48 (1H, m, hidden under DMSO signal), 3.15-3.21 (2H, m), 3.92 (3H, s), 4.20 (2H, q, J=7.2 Hz), 4.53-4.58 (2H, m), 4.70 (2H, s), 7.22-7.27 (2H, m), 7.31-7.36 (2H, m), 8.28 (1H, s).

MS m/z: 505 (M+1)

GTPγS(IC₅₀ μM): 0.009

### Example 12

### Ethyl 6-(4-{[(4-chlorobenzyl)sulfonyl]carbamoyl}piperidin-1-yl)-5-cyano-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(4-chlorophenyl)methanesulfonamide (24 mg, 0.115 mmol). Yield: 15.7 mg (30%).

¹H NMR (600 MHz, DMSO-d₆): δ 1.26 (3H, t, J=7.2 Hz), 1.60-1.68 (2H, m), 1.82-1.87 (2H, m), 2.40-2.48 (1H, m, hidden under DMSO signal), 3.15-3.21 (2H, m), 3.92 (3H, s), 4.20 (2H, q, J=7.2 Hz), 4.53-4.58 (2H, m), 4.71 (2H, s), 7.30-7.33 (2H, m), 7.47-7.50 (2H, m), 8.28 (1H, s).

MS m/z: 521 (M+1)

GTPγS(IC₅₀ μM): 0.009

### Example 13

### Ethyl 5-cyano-2-methoxy-6-[4-({[4-(trifluoromethyl)benzyl]sulfonyl}carbamoyl)piperidin-1-yl]nicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-[4-(trifluoromethyl)phenyl]methanesulfonamide (28 mg, 0.115 mmol). Yield: 18.9 mg (34%).

¹H NMR (600 MHz, DMSO-d₆): δ 1.26 (3H, t, J=7.2 Hz), 1.60-1.68 (2H, m), 1.82-1.87 (2H, m), 2.40-2.48 (1H, m, hidden under DMSO signal), 3.15-3.21 (2H, m), 3.92 (3H, s), 4.20 (2H, q, J=7.2 Hz), 4.53-4.58 (2H, m), 4.84 (2H, s), 7.53 (2H, d, J=8.0 Hz), 7.79 (2H, d, J=8.0 Hz), 8.28 (1H, s).

MS m/z: 555 (M+1)

GTPγS(IC₅₀ μM): 0.019

### Example 14

### Ethyl 5-cyano-6-(4-{[(3,4-difluorobenzyl)sulfonyl]carbamoyl}piperidin-1-yl)-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(3,4-difluorophenyl)methanesulfonamide (24 mg, 0.115 mmol). Yield: 18.3 mg (35%).

¹H NMR (600 MHz, DMSO-d₆): δ 1.26 (3H, t, J=7.2 Hz), 1.60-1.68 (2H, m), 1.82-1.87 (2H, m), 2.40-2.48 (1H, m, hidden under DMSO signal), 3.15-3.21 (2H, m), 3.92 (3H, s), 4.20 (2H, q, J=7.2 Hz), 4.53-4.58 (2H, m), 4.72 (2H, s), 7.12-7.16 (1H, m), 7.34-7.40 (1H, m), 7.46-7.52 (1H, m), 8.28 (1H, s).

MS m/z: 523 (M+1)

GTPγS(IC₅₀ μM): 0.013

### Example 15

### Ethyl 5-cyano-6-(4-{[(2,4-dichlorobenzyl)sulfonyl]carbamoyl}piperidin-1-yl)-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(2,4-dichlorophenyl)methanesulfonamide (28 mg, 0.115 mmol). Yield: 20.6 mg (37%).

¹H NMR (600 MHz, DMSO-d₆): δ 1.26 (3H, t, J=7.2 Hz), 1.62-1.70 (2H, m), 1.88-1.93 (2H, m), 2.40-2.48 (1H, m, hidden under DMSO signal), 3.16-3.23 (2H, m), 3.92 (3H, s), 4.20 (2H, q, J=7.2 Hz), 4.54-4.61 (2H, m), 4.86 (2H, s), 7.46-7.48 (1H, m), 7.52-7.54 (1H, m), 7.71-7.73 (1H, m), 8.28 (1H, s).

MS m/z: 555 (M+1)

GTPγS(IC₅₀ μM): 0.022

### Example 16

### Ethyl 5-cyano-6-(4-{[(2,4-difluorobenzyl)sulfonyl]carbamoyl}piperidin-1-yl)-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(2,4-difluorophenyl)methanesulfonamide (25 mg, 0.115 mmol). Yield: 20.7 mg (39%).

¹H NMR (600 MHz, DMSO-d₆): δ 1.26 (3H, t, J=7.2 Hz), 1.60-1.68 (2H, m), 1.82-1.87 (2H, m), 2.40-2.48 (1H, m, hidden under DMSO signal), 3.15-3.21 (2H, m), 3.92 (3H, s), 4.20 (2H, q, J=7.2 Hz), 4.53-4.58 (2H, m), 4.72 (2H, s), 7.12-7.16 (1H, m), 7.34-7.40 (1H, m), 7.46-7.52 (1H, m), 8.28 (1H, s).

MS m/z: 523 (M+1)

GTPγS(IC₅₀ μM): 0.008

### Example 17

### Ethyl 6-(4-{[(2-chloro-4-fluorobenzyl)sulfonyl]carbamoyl}piperidin-1-yl)-5-cyano-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(2-chloro-4-fluorophenyl)methanesulfonamide (27 mg, 0.115 mmol). Yield: 21.1 mg (39%).

¹H NMR (600 MHz, DMSO-d₆): δ 1.26 (3H, t, J=7.2 Hz), 1.62-1.71 (2H, m), 1.87-1.93 (2H, m), 2.40-2.48 (1H, m, hidden under DMSO signal), 3.16-3.23 (2H, m), 3.92 (3H, s), 4.20 (2H, q, J=7.2 Hz), 4.54-4.60 (2H, m), 4.84 (2H, s), 7.30-7.35 (1H, m), 7.49-7.56 (2H, m), 8.28 (1H, s).

MS m/z: 539 (M+1)

GTPγS(IC₅₀ μM): 0.024

### Example 18

### Ethyl 6-(4-{[(4-chloro-2-fluorobenzyl)sulfonyl]carbamoyl}piperidin-1-yl)-5-cyano-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(4-chloro-2-fluorophenyl)methanesulfonamide (27 mg, 0.115 mmol). Yield: 13.9 mg (26%).

¹H NMR (600 MHz, DMSO-d₆): δ 1.26 (3H, t, J=7.2 Hz), 1.61-1.69 (2H, m), 1.85-1.91 (2H, m), 2.40-2.48 (1H, m, hidden under DMSO signal), 3.16-3.23 (2H, m), 3.92 (3H, s), 4.20 (2H, q, J=7.2 Hz), 4.54-4.60 (2H, m), 4.76 (2H, s), 7.36-7.39 (1H, m), 7-.42-7.46 (1H, m), 7.51-7.55 (1H, m), 8.28 (1H, s).

MS m/z: 539 (M+1)

GTPγS(IC₅₀ μM): 0.01

### Example 19

### Ethyl 5-cyano-6-(4-{[(2,3-difluorobenzyl)sulfonyl]carbamoyl}piperidin-1-yl)-2-methoxynicotinate

Prepared according to the procedure described in Example 7(e) using 1-[3-Cyano-5-(ethoxycarbonyl)-6-methoxypyridin-2-yl]piperidine-4-carboxylic acid (33.3 mg, 0.1 mmol) and 1-(2,3-difluorophenyl)methanesulfonamide (24 mg, 0.115 mmol). Yield: 15.3 mg (29%).

¹H NMR (600 MHz, DMSO-d₆): δ 1.26 (3H, t, J=7.2 Hz), 1.62-1.70 (2H, m), 1.85-1.91 (2H, m), 2.40-2.48 (1H, m, hidden under DMSO signal), 3.16-3.23 (2H, m), 3.92 (3H, s), 4.20 (2H, q, J=7.2 Hz), 4.53-4.58 (2H, m), 4.82 (2H, s), 7.20-7.25 (1H, m), 7.25-7.30 (1H, m), 7.46-7.52 (1H, m), 8.28 (1H, s).

MS m/z: 523 (M+1)

GTPγS(IC₅₀ μM): 0.031

### Example 20

### Ethyl 5-cyano-2-methoxy-6-{3-[(phenylsulfonyl)carbamoyl]azetidin-1-yl}nicotinate

A solution of DIPEA (129.2 mg, 1 mmol), 1-[3-cyano-5-(ethoxycarbonyl)-6-methoxypyridin-2-yl]azetidine-3-carboxylic acid (30.5 mg, 0.1 mmol) and PyBrop (70 mg, 0.15 mmol) in DCM (2 mL) was added to benzenesulfonamide (18 mg, 0.115 mmol) and the mixture was stirred at r.t for 2 hours. The solvent was evaporated and the crude product purified according the purification Method A (See General Experimental Procedure) to give the desired product. Yield: 16.8 mg (38%).

¹H NMR (600 MHz, DMSO-d₆): δ 1.19 (3H, t, J=7.0 Hz), 3.51-3.57 (1H, m), 3.81 (3H, s), 4.11 (2H, q, J=7.0 Hz), 4.15 (2H, m), 4.35 (2H, m), 7.57-7.61 (2H, m), 7.66-7.70 (1H, m), 7.88-7.91 (2H, m), 8.15 (1H, s).

MS m/z: 445 (M+1), 443 (M−1)

GTPγS(IC₅₀ μM): 0.102

### Example 21

### Ethyl 5-cyano-6-(3-{[(2-fluorobenzypsulfonyl]carbamoyl}azetidin-1-yl)-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0.1 mmol) and 1-(2-fluorophenyl)methanesulfonamide (22 mg, 0.115 mmol). Yield: 21.7 mg (45%).

¹H NMR (600 MHz, DMSO-d₆): δ 1.19 (3H, t, J=7.0 Hz), 3.51-3.57 (1H, m), 3.81 (3H, s), 4.11 (2H, q, J=7.0 Hz), 4.15 (2H, m), 4.35 (2H, m), 7.57-7.61 (2H, m), 7.66-7.70 (1H, m), 7.88-7.91 (2H, m), 8.15 (1H, s).

MS m/z: 445 (M+1), 443 (M−1)

GTPγS(IC₅₀ μM): 0.015

### Example 22

### Ethyl 6-(3-{[(2-chlorobenzyl)sulfonyl]carbamoyl}azetidin-1-yl)-5-cyano-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0.1 mmol) and 1-(2-chlorophenyl)methanesulfonamide (24 mg, 0.115 mmol). Yield: 25.7 mg (52%).

¹H NMR (600 MHz, DMSO-d₆): δ 1.21 (3H, t, J=7.0 Hz), 3.52-3.59 (1H, m), 3.86 (3H, s), 4.13 (2H, q, J=7.0 Hz), 4.25-4.48 (4H, m), 4.85 (2H, s), 7.32-7.40 (2H, m), 7.45-7.49 (2H, m), 8.20 (1H, s).

MS m/z: 493 (M+1), 491 (M−1)

GTPγS(IC₅₀ μM): 0.012

### Example 23

### Ethyl 5-cyano-6-(3-{[(3-fluorobenzyl)sulfonyl]carbamoyl}azetidin-1-yl)-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0.1 mmol) and 1-(3-fluorophenyl)methanesulfonamide (22 mg, 0.115 mmol). Yield: 23.2 mg (49%).

¹H NMR (600 MHz, DMSO-d₆): δ 1.21 (3H, t, J=7.0 Hz), 3.48-3.55 (1H, m), 3.86 (3H, s), 4.13 (2H, q, J=7.0 Hz), 4.22-4.43 (4H, m), 4.73 (2H, s), 7.12-7.15 (2H, m), 7.16-7.21 (1H, m), 7.35-7.41 (1H, m), 8.20 (1H, s).

MS m/z: 477 (M+1), 475 (M−1)

GTPγS(IC₅₀ μM): 0.044

### Example 24

### Ethyl 5-cyano-6-(3-{[(4-fluorobenzyl)sulfonyl]carbamoyl}azetidin-1-yl)-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0 1 mmol) and 1-(4-fluorophenyl)methanesulfonamide (22 mg, 0.115 mmol). Yield: 22.4 mg (47%).

¹H NMR (600 MHz, DMSO-d₆): δ 1.21 (3H, t, J=7.0 Hz), 3.48-3.54 (1H, m), 3.86 (3H, s), 4.13 (2H, q, J=7.0 Hz), 4.20-4.44 (4H, m), 4.70 (2H, s), 7.14-7.19 (2H, m), 7.32-7.36 (2H, m), 8.20 (1H, s).

MS m/z: 477 (M+1), 475 (M−1).

GTPγS(IC₅₀ μM): 0.009

### Example 25

### Ethyl 6-(3-{[(4-chlorobenzyl)sulfonyl]carbamoyl}azetidin-1-yl)-5-cyano-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0.1 mmol) and 1-(4-chlorophenyl)methanesulfonamide (24 mg, 0.115 mmol). Yield: 18.6 mg (38%).

¹H NMR (600 MHz, DMSO-d₆): δ 1.21 (3H, t, J=7.0 Hz), 3.48-3.55 (1H, m), 3.86 (3H, s), 4.13 (2H, q, J=7.0 Hz), 4.20-4.45 (4H, m), 4.70 (2H, s), 7.30-7.33 (2H, m), 7.38-7.41 (2H, m), 8.20 (1H, s).

MS m/z: 493 (M+1), 491 (M−1)

GTPγS(IC₅₀ μM): 0.006

### Example 26

### Ethyl 5-cyano-2-methoxy-6-[3-({[4-(trifluoromethyl)benzyl]sulfonyl}carbamoyl)azetidin-1-yl]nicotinate

Prepared according to the procedure described in Example 20 using 1-[3-cyano-5-(ethoxycarbonyl)-6-methoxypyridin-2-yl]azetidine-3-carboxylic acid (30.5 mg, 0.1 mmol) and 1-[4-(trifluoromethyl)phenyl]methanesulfonamide (27 mg, 0.115 mmol). Yield: 19.8 mg (38%).

¹H NMR (600 MHz, DMSO-d₆): δ 1.21 (3H, t, J=7.0 Hz), 3.48-3.55 (1H, m), 3.86 (3H, s), 4.13 (2H, q, J=7.0 Hz), 4.20-4.45 (4H, m), 4.83 (2H, s), 7.52-7.55 (2H, m), 7.69-7.74 (2H, m), 8.19 (1H, s).

MS m/z: 527 (M+1), 525 (M−1)

GTPγS(IC₅₀ μM): 0.012

### Example 27

### Ethyl 5-cyano-6-(3-{[(3,4-difluorobenzyl)sulfonyl]carbamoyl}azetidin-1-yl)-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0.1 mmol) and 1-(3,4-difluorophenyl)methanesulfonamide (24 mg, 0.115 mmol). Yield: 13.9 mg (28%).

¹H NMR (600 MHz, DMSO-d₆): δ 1.21 (3H, t, J=7.0 Hz), 3.48-3.55 (1H, m), 3.86 (3H, s), 4.13 (2H, q, J=7.0 Hz), 4.19-4.43 (4H, m), 4.72 (2H, s), 7.14-7.18 (1H, m), 7.35-7.44 (2H, m), 8.19 (1H, s).

MS m/z: 495 (M+1), 493 (M−1)

GTPγS(IC₅₀ μM): 0.035

### Example 28

### Ethyl 5-cyano-6-(3-{[(2,4-dichlorobenzyl)sulfonyl]carbamoyl}azetidin-1-yl)-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0.1 mmol) and 1-(2,4-dichlorophenyl)methanesulfonamide (28 mg, 0.115 mmol). Yield: 20.9 mg (40%).

¹H NMR (600 MHz, DMSO-d₆): δ 1.21 (3H, t, J=7.0 Hz), 3.52-3.59 (1H, m), 3.86 (3H, s), 4.13 (2H, q, J=7.0 Hz), 4.26-4.48 (4H, m), 4.85 (2H, s), 7.44-7.51 (2H, m), 7.64-7.67 (1H, m), 8.20 (1H, s).

MS m/z: 528 (M+1), 526 (M−1)

GTPγS(IC₅₀ μM): 0.005

### Example 29

### Ethyl 5-cyano-6-(3-{[(2,4-difluorobenzyl)sulfonyl]carbamoyl}azetidin-1-yl)-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0 1 mmol) and 1-(2,4-difluorophenyl)methanesulfonamide (24 mg, 0.115 mmol). Yield: 26 mg (53%).

¹H NMR (600 MHz, DMSO-d₆): δ 1.21 (3H, t, J=7.0 Hz), 3.51-3.58 (1H, m), 3.86 (3H, s), 4.13 (2H, q, J=7.0 Hz), 4.24-4.48 (4H, m), 4.74 (2H, s), 7.08-7.13 (1H, m), 7.23-7.29 (1H, m), 7.45-7.50 (1H, m), 8.20 (1H, s).

MS m/z: 495 (M+1), 493 (M−1)

GTPγS(IC₅₀ μM): 0.01

### Example 30

### Ethyl 6-(3-{[(2-chloro-4-fluorobenzyl)sulfonyl]carbamoyl}azetidin-1-yl)-5-cyano-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0.1 mmol) and 1-(2-chloro-4-fluorophenyl)methanesulfonamide (24 mg, 0.115 mmol). Yield: 15 mg (29%).

¹H NMR (400 MHz, DMSO-d₆): δ 1.25 (3H, t, J=7.1 Hz), 3.54-3.63 (1H, m), 3.90 (3H, s), 4.17 (2H, q, J=7.1 Hz), 4.30-4.50 (4H, m), 4.86 (2H, s), 7.25-7.33 (1H, m), 7.49-7.60 (2H, m), 8.23 (1H, s), 12.02 (1H, br s).

MS m/z: 511 (M+1), 509 (M−1)

GTPγS(IC₅₀ μM): 0.009

### Example 31

### Ethyl 6-(3-{[(4-chloro-2-fluorobenzyl)sulfonyl]carbamoyl}azetidin-1-yl)-5-cyano-2-methoxynicotinate

Prepared according to the procedure described in Example 20 using 1-[3-cyano-5-(ethoxycarbonyl)-6-methoxypyridin-2-yl]azetidine-3-carboxylic acid (30.5 mg, 0.1 mmol) and 1-(4-chloro-2-fluorophenyl)methanesulfonamide (26 mg, 0.115 mmol). Yield: 16.3 mg (32%).

¹H NMR (600 MHz, DMSO-d₆): δ 1.21 (3H, t, J=7.0 Hz), 3.51-3.58 (1H, m), 3.86 (3H, s), 4.13 (2H, q, J=7.0 Hz), 4.26-4.48 (4H, m), 4.75 (2H, s), 7.29-7.33 (1H, m), 7.42-7.47 (2H, m), 8.20 (1H, s).

MS m/z: 511 (M+1), 509 (M−1)

GTPγS(IC₅₀ μM): 0.005

### Example 32

### Ethyl 5-cyano-6-(3-{[(2,3-difluorobenzyl)sulfonyl]carbamoyl}azetidin-1-yl)-2-methoxynicotinate

Prepared essentially according to the procedure described in Example 20 using 1-[3-cyano-5-(ethoxycarbonyl)-6-methoxypyridin-2-yl]azetidine-3-carboxylic acid (30.5 mg, 0.1 mmol) and 1-(2,3-difluorophenyl)methanesulfonamide (26 mg, 0.115 mmol). Yield: 22.1 mg (44%).

¹H NMR (600 MHz, DMSO-d₆): δ 1.21 (3H, t, J=7.0 Hz), 3.52-3.59 (1H, m), 3.86 (3H, s), 4.13 (2H, q, J=7.0 Hz), 4.27-4.49 (4H, m), 4.82 (2H, s), 7.19-7.26 (2H, m), 7.41-7.47 (1H, m), 8.20 (1H, s).

MS m/z: 493 (M+1), 495 (M−1)

GTPγS(IC₅₀ μM): 0.083

### Example 33

### Ethyl 6-{3-[(benzylsulfonyl)carbamoyl]azetidin-1-yl}-5-cyano-2-(ethoxymethyl)nicotinate

### (a) 1-(tert-butoxycarbonyl)azetidine-3-carboxylic acid

(Boc)₂O (25.535 g, 117 mmol) dissolved in Me0H (70 mL) was added dropwise during 20 minutes to a stirred slurry of azetidine-3-carboxylic acid (10.11 g, 100 mmol) and Et₃N (27.8 mL, 200 mmol) in MeOH (105 mL) at r.t (mildly exotermic reaction) and the mixture was stirred over night (18 hours). The reaction was evaporated to dryness and THF (120 mL) was added and evaporated to give crude 1-(tert-butoxycarbonyl)azetidine-3-carboxylic acid which was used without further purification in the next step. Yield: 25.89 g (128%)

¹H NMR (400 MHz, CDCl₃) δ 1.43 (9H, s), 3.21-3.34 (1H, m), 4.00-4.13 (4H, m).

### (b) tert-butyl 3-[(benzylsulfonyl)carbamoyl]azetidine-1-carboxylate

TBTU (33.71 g, 105 mmol) and TEA (30.3 g, 300 mmol) was added to a solution of 1-(tert-butoxycarbonyl)azetidine-3-carboxylic acid from above (25.89 g, assumed to contain 100 mmol) and the reaction was stirred at r.t for 30 minutes. 1-phenylmethanesulfonamide (17.97 g, 105 mmol) and LiCl (1.844 g, 43.5 mmol) was added and the stirring was continued at r.t over night (23 hours). The reaction was concentrated to about ⅓ was left and EtOAc (500 mL) was added and the organic phase was washed with 2 M HCl (1×150 mL, 2×50 mL), water (2×50 mL). Drying (MgSO₄), filtration and evaporation of the solvent gave a brown powder (48. 6 g). The powder was slurried in 150 mL MTBE and stirred 3 hours. The solids was filtered off and washed with MTBE (40 mL). This procedure was repeated twice with 100 mL MTBE (washing with 25 mL) to give a brownish powder (33 g) still containing some HOBt. The powder was dissolved in about 100 mL warm EtOH and water (130 mL) was added to induce a crystallisation of the product. The crystals was filtered off and dried to give pure tert-butyl 3-[(benzylsulfonyl)carbamoyl]azetidine-1-carboxylate as an off white powder. Yield: 25.4 g (71%).

¹H NMR (400 MHz, DMSO-d₆) δ 1.39 (9H, s), 3.30 (1H, m, overlapping with the water signal in DMSO), 3.78-3.95 84H, m), 4.73 (2H, s), 7.28-7.34 (2H, m), 7.36-7.41 (3H, m), 11.71 (1H, br s).

MS m/z: 353 (M−1).

### (c) N-(benzylsulfonyl)azetidine-3-carboxamide

tert-butyl 3-[(benzylsulfonyl)carbamoyl]azetidine-1-carboxylate (25.4 g, 71.7 mmol) was added to HCOOH (300 mL) at r.t and the reaction was stirred over night (22 hours). The formic acid was removed in vaccuo, water (40 mL) was added and removed in vaccuo. Water (130 mL) was added to the residue followed by NH₄OH (aq) until pH reached 7.4 when a crystallization started. The crystals was filtered off and dried to give pure N-(benzylsulfonyl)azetidine-3-carboxamide as a white solid. Yield 15.73 g (86%).

¹H NMR (400 MHz, DMSO-d₆) δ 3.22 (1H, m), 3.87-3.96 (4H, m), 4.28 (2H, s), 7.20-7.32 (5H, m).

MS m/z: 255 (M+1)

### (d) Ethyl 2-(chloromethyl)-5-cyano-6-oxo-1,6-dihydropyridine-3-carboxylate

A mixture of ethyl 4-chloro-3-oxobutanoate (10 g, 60.75 mmol), acetic anhydride (27.3 g, 267.3 mmol) and triethyloethoformate was heated at 120° C. (bath temperature) for 3 hours. The dark mixture was concentrated in vacuo and co-evaporated once with toluene (50 mL). Heptane (50 mL) was added to precipitate the product and removed in vacuo. The crude material was dissolved in EtOH (50 mL).

In a separate flask, sodium ethoxide (50 mL, 60.75 mmol, prepared by reaction of sodium with EtOH (50 mL)) was added dropwise to a cold (<5° C.) solution of 2-cyanoacetamide (5.11 g, 60.75 mmol) in EtOH (50 mL) and the mixture was stirred for 30 minutes after which the solution of the crude material from above was added over 10 minutes and the stirring was continued at r.t over night. The solid formed was isolated by filtration and washed with MTBE (50 mL). Drying of the filtrate gave ethyl 2-(chloromethyl)-5-cyano-6-oxo-1,6-dihydropyridine-3-carboxylate as a beige solid. Yield: 8.15 g (56%).

¹H NMR (500 MHz, DMSO-d₆) δ 1.27 (3H, t, J=7.0 Hz), 4.16 (2H, q, J=7.0 Hz), 4.75 (2H, s), 8.02 (1H, s)

### (e) Ethyl 6-chloro-2-(chloromethyl)-5-cyanonicotinate

DMF (0.076 g, 1.04 mmol) was added to a stirred slurry of ethyl 2-(chloromethyl)-5-cyano-6-oxo-1,6-dihydropyridine-3-carboxylate (1.00 g, 4.16 mmol) and oxalyl chloride (10.55 g, 83.11 mmol) at r.t (immediate gas evolution was observed) . The mixture was heated to 70° C. for 4 hours and then at 50° C. overnight. The mixture was diluted with butyronitrile and evaporated (twice with 20 mL) to remove excess oxalylchloride. The residue was partioned between butyronitrile (50 mL) and water (50 ml) and the water phase was acidified with concentrated HCl (0.5 mL) followed by addition of MgCl₂(aq) to is aid phase separation. The organic phase was separated and washed with water (25 mL), 20% Na₂CO₃(aq) (0.5 mL), MgCl₂(aq) (10 mL) and dried (MgSO₄). The crude material was purified by chromatography on silica (Eluent: a gradient of 90:10 to 40:60 to give the desired product as a coulorless solid. Yield: 2.56 g (61%).

¹H NMR (500 MHz, DMSO-d₆) δ 1.36 (3H, t, J=7.1 Hz), 4.38 (2H, q, J=7.1 Hz), 5.09 (2H, s), 8.90 (1H, s)

MS m/z: 258 (M−1)

### (f) Ethyl 6-{3-[(benzylsulfonyl)carbamoyl]azetidin-1-yl}-2-(chloromethyl)-5-cyanonicotinate

A microwave vial was charged with 6-chloro-2-(chloromethyl)-5-cyanonicotinate (417 mg, 1.61 mmol), N-(benzylsulfonyl)azetidine-3-carboxamide (429 mg, 1.69 mmol), TEA (407 mg, 4.02 mmol) and EtOH (5 mL) and heated to 100° C. for 10 minutes. The mixture was diluted with DCM (25 mL), water (10 mL) and concentrated HCl (226 μL). The phases was separated and the organic phase dried (MgSO4) and evaporated to give the desired product as a pale yellow solid. Yield: 590 mg (77%).

¹H NMR (500 MHz, DMSO) δ 1.32 (3H, t, J=7.1 Hz), 3.55-3.63 (1H, m), 4.28 (2H, q, J=7.1 Hz), 4.31-4.53 (4H, m), 4.76 (2H, s), 4.95 (2H, s), 7.31-7.43 (5H, m), 8.42 (1H, s), 11.83 (1H, s)

### (g) Ethyl 6-{3-[(benzylsulfonyl)carbamoyl]azetidin-1-yl}-5-cyano-2-(ethoxymethyl)nicotinate

A microwave vial was charged with ethyl 6-{3-[(benzylsulfonyl)carbamoyl]azetidin-1-yl}-2-(chloromethyl)-5-cyanonicotinate (50 mg, 0.105 mmol), Cs₂CO₃ (68.3 mg, 0.210 mmol), sodium iodide (15.7 mg, 0.105 mmol) and EtOH (1.0 mL) and the mixture was heated to 100° C. in a microwave oven, single node heating, for 15 minutes and at r.t. overnight. The reaction was quenched by adding AcOH (0.024 mL, 0.419 mmol) and evaporated. The residue was partitioned between DCM (5 mL) nas water (5 mL). The phases were separated and the organic phase evaporated to give a crude product which was purified according to purification Method A (See General Experimental Procedure) to give the is desired product. Yield: 11.1 mg (21%).

¹H NMR (600 MHz, DMSO-d₆) δ 1.09 (3H, t, J=7.0 Hz), 1.27 (3H, t, J=7.0 Hz), 3.47-3.56 (2H, m), 3.49 (2H, q, J=7.2 Hz), 4.21 (2H, q, J=7.2 Hz), 4.25-4.33 (2H, m), 4.36-4.43 (2H, m), 4.68 (2H, s), 4.70 (2H, br s), 7.29-7.37 (5H, m), 8.27 (1H, s)

MS m/z: 487 (M+1)

GTPγS(IC₅₀ μM): 0.069

### Example 34

### Ethyl 6-{4-[(benzylsulfonyl)carbamoyl]piperidin-1-yl}-5-cyano-2-(ethoxymethyl)nicotinate

### (a) Ethyl 6-{4-[(benzylsulfonyl)carbamoyl]piperidin-1-yl}-2-(chloromethyl)-5-cyanonicotinate

A microwave vial was charged with ethyl 6-chloro-2-(chloromethyl)-5-cyanonicotinate (540 mg, 2.08 mmol), N-(benzylsulfonyl)piperidine-4-carboxamide (618 mg, 2.19 mmol), TEA (527 mg, 5.21 mmol), EtOH (0.5 mL) and heated to 100° C. for 10 minutes using a microwave oven. The solvent was removed in vacuo and the residue was partioned between iPrOAc (20 mL) and aq HCl (435 μL, 37% HCl in 15 mL water). The aqeous phase was separated and re-extracted with iPrOAc (10 mL).The combined organic phases was washed with aqueous MgCl₂ (10 mL), dried (MgSO₄) and evaporated to give the product which was used without further purification. Yield: 929 mg (88%).

¹H NMR (500 MHz, CDCl₃) δ 1.41 (3H, t, J=7.1 Hz), 1.75-1.94 (4H, m), 2.50 (1H, ddd, J=15.0, 10.8, 4.1 Hz), 3.19 (2H, dd, J=25.1, 2.3 Hz), 4.37 (2H, q, J=7.2 Hz), 4.63 (2H, s), 4.71 (2H, d, J=13.7 Hz), 4.98 (2H, s), 7.27-7.45 (5H, m), 8.41 (1H, s).

### (b) ethyl 6-{4-[(benzylsulfonyl)carbamoyl]piperidin-1-yl}-5-cyano-2-(ethoxymethyl)nicotinate

A microwave vial was charged with ethyl 6-{4-[(benzylsulfonyl)carbamoyl]piperidin-1-yl}-2-(chloromethyl)-5-cyanonicotinate (25 mg, 0.05 mmol), Cs₂CO₃ (32.3 mg, 0.099 mmol), sodium iodide (7.4 mg, 0.05 mmol) and EtOH (0.5 mL) and the mixture was heated to 100° C. in a microwave oven, single node heating, for 15 minutes and at r.t. over night. The solvent was evaporated and the residue was partitioned between DCM (5 mL) nas water (5 mL). The phases were separated and the organic phase evaporated to give a crude product which was purified according to purification Method A (See General Experimental Procedure) to give the desired product. Yield: 6.6 mg (24%).

¹H NMR (600 MHz, DMSO-d₆) δ 1.10 (3H, t, J=7.2 Hz), 1.27 (3H, t, J=7.2 Hz), 1.56-1.66 (2H, m), 1.78-1.84 (2H, m), 3.11-3.18 (2H, m), 3.49 (2H, q, J=7.2 Hz), 4.22 (2H, q, J=7.2 Hz), 4.50-4.56 (2H, m), 4.65 (2H, s), 4.70 (2H, s), 7.23-7.29 (2H, m), 7.33-7.39 (3H, m), 8.30 (1H, s)

MS m/z: 515 (M+1)

GTPγS(IC₅₀ μM): 0.034

### Example 35

### Ethyl 2-[(benzyloxy)methyl]-6-{3-[(benzylsulfonyl)carbamoyl]azetidin-1-yl}-5-cyanonicotinate

### (a) Ethyl 2-[(benzyloxy)methyl]-5-cyano-6-oxo-1,6-dihydropyridine-3-carboxylate

1,1-dimethoxy-N,N-dimethylmethanamine (2.52 g, 21.2 mmol) was added to neat ethyl 4-(benzyloxy)-3-oxobutanoate (Yasohara Y et al, Tetrahedron assymetry, 12(2001) pp. 1713-18.) and the reaction mixture was stirred over night. The volatiles were evaporated and the residue co-evaporated once with toluene (20 mL) and dissolved in EtOH (25 mL). This solution is used as such below.

A solution of sodium ethoxide in EtOH (487 mg Na in 25 mL EtOH) was added dropwise (during 10 minutes) to a solution of 2-cyanoacetamide (1.78 g, 21.2 mmol) in EtOH (25 mL). The solution from above was added via a dropping funnel (slightly exotermic) and the dropping funnel was rinsed with EtOH (25 mL). A pale yellow precipitate of product was formed during the reaction. The slurry was stirred at r.t over night and quenched with AcOH (1.21 mL, 21.16 mL). The solid was isolated by filtration and the filter cake washed with MTBE (50 mL) to give 1.6 g of a crude product. The liquors was concentrated to give a pale solid. The solids were recombined and slurried in water (100 mL)+1 M HCl (25 mL). The mixture was stirred for about 30 minutes and the solid was isolated by filtration. The wet solid was slurried in toluene (200 mL) and concentrated in vacuo and re-slurried in IPA (100 mL) and filtered to give the desired product. Yield: 3.74 g, (57%).

¹H NMR (500 MHz, DMSO-d₆) δ 1.26 (3H, t, J=7.1 Hz), 4.21 (2H, q, J=7.1 Hz), 4.62 (2H, s), 4.85 (2H, s), 7.27-7.41 (5H, m), 8.46 (1H, s), 12.52 (1H, s)

MS m/z: 313 (M+1), 311 (M−1)

### (b) Ethyl 2-[(benzyloxy)methyl]-6-chloro-5-cyanonicotinate

Oxalyl chloride (6.10 g, 48 mmol) dissolved in DCM (20 mL) was added over 10 minutes to a suspension of ethyl 2-[(benzyloxy)methyl]-5-cyano-6-oxo-1,6-dihydropyridine-3-carboxylate (3.00 g, 9.61 mmol) and DMF (702 mg, 9.61 mmol) in DCM (30 mL) and the mixture was stirred at r.t for 3 hours (still remaining starting material). A one mL aliquote was taken and heated to 100° C. for 30 minutes in a microwave oven (LC-MS showed essentially complete conversion). The remaining material was heated the same way in three batches. The batches was recombined and quenched by 1 M NaOH and diluted with DCM (50 mL). The phases were separated and the black organic phase concentrated. The crude product was purified by flash column chromatography (using a gradient of 90:10 to 60:40 heptane/EtOAc) to give the desired product. Yield: 1.70 g (53%).

¹H NMR (500 MHz, DMSO-d₆) δ1.29 (3H, t, J=7.1 Hz), 4.29 (2H, q, J=7.1 Hz), 4.55 (2H, s), 4.90 (2H, s), 7.27-7.37 (5H, m), 8.77 (1H, s)

MS m/z: 333 (M+1), 331 (M−1)

### (c) Ethyl 2-[(benzyloxy)methyl]-6-{3-[(benzylsulfonyl)carbamoyl]azetidin-1-yl}-5-cyanonicotinate

A microwave vial was charged with ethyl 2-[(benzyloxy)methyl]-6-chloro-5-cyanonicotinate (200 mg, 0.605 mmol), N-(benzylsulfonyl)azetidine-3-carboxamide (161 mg, 0.635 mmol), DIPEA (195 mg, 1.512 mmol) and EtOH (2 mL) and the mixture was heated to 100° C. in a microwave oven, single node heating, for 10 minutes. The reaction mixture was diluted with iPrOAc (10 mL), 1 M HCl (1.5 mL) and water (8.5 mL). The organic phase was separated and concentrated to give a pale solid. The solid was slurried in IPA at 40° C. The solid was isolated by filtration to give the desired compound. Yield: 263 mg, (79%).

¹H NMR (500 MHz, DMSO-d₆) δ 1.28 (3H, t, J=7.1 Hz), 3.54-3.63 (1H, m), 4.23 (2H, q, J=7.1 Hz), 4.29-4.39 (2H, m), 4.39-4.50 (2H, m), 4.59 (2H, s), 4.76 (2H, s), 4.84 (2H, s), 7.25-7.42 (10H, m), 8.32 (1H, s), 11.83 (1H, s)

MS m/z: 549 (M+1), 547(M−1)

GTPγS(IC₅₀ μM): 0.089

### Example 36

### Ethyl 2-[(benzyloxy)methyl]-6-{4-[(benzylsulfonyl)carbamoyl]piperidin-1-yl}-5-cyanonicotinate

A microwave vial was charged with ethyl 2-[(benzyloxy)methyl]-6-chloro-5-cyanonicotinate (200 mg, 0.605 mmol), N-(benzylsulfonyl)piperidine-4-carboxamide (171 mg, 0.635 mmol), DIPEA (195 mg, 1.512 mmol) and EtOH (2 mL) and the mixture was heated to 100° C. in a microwave oven, single node heating, for 10 minutes. The reaction mixture was diluted with iPrOAc (10 mL), 1 M HCl (1.5 mL) and water (8.5 mL). The solid was isolated by filtration and washed with IPA (10 mL) to give the desired product as a colourless solid.

¹H NMR (500 MHz, DMSO-d₆) δ 1.29 (3H, t, J=7.1 Hz), 1.59-1.71 (2H, m), 1.78-1.87 (2H, m), 2.56-2.64 (1H, m), 3.17 (2H, t, J=11.8 Hz), 4.24 (2H, q, J=7.1 Hz), 4.53-4.62 (2H, m), 4.59 (2H, s), 4.70 (2H, s), 4.86 (2H, s), 7.26-7.43 (10H, m), 8.35 (1H, s), 11.60 (1H, s)

MS m/z: 577 (M+1), 575 (M−1)

GTPγS(IC₅₀ μM): 0.055

### Example 37

### Ethyl 6-{3-[(benzylsulfonyl)carbamoyl]azetidin-1-yl}-5-cyano-2-(hydroxymethyl)nicotinate

### (a) Ethyl 5-cyano-2-{[(3,4-dimethoxybenzyl)oxy]methyl}-6-[(methylsulfonyl)oxy]nicotinate

DIPEA (260 mg, 2.01 mmol) and mesylchloride (230 mg, 2.01 mmol dissolved in DCM 1 mL) were added to a solution of ethyl 5-cyano-2-{[(3,4-dimethoxybenzyl)oxy]methyl}-6-oxo-1,6-dihydropyridine-3-carboxylate (500 mg, 1.34 mmol) in DCM (4 mL) and the reaction was stirred at r.t for about 10 minutes. Water (5 mL) was added and the waterphase was acidified with 1 M HCl to pH<2. The organic phase was separated and evaporated to give the desired product. Yield: 670 mg (99%).

¹H NMR (500 MHz, DMSO-d₆) δ 1.31 (3H, t, J=7.5 Hz), 3.74 (6H, s), 3.86 (3H, s), 4.31 (2H, q, J=7.1 Hz), 4.51 (2H, s), 4.94 (2H, s), 6.82-6.87 (1H, m), 6.88-6.93 (2H, m), 8.88 (1H, s)

MS m/z: 451 (M+1), 468 (M+NH₄)

### (b) Ethyl 6-{3-[(benzylsulfonyl)carbamoyl]azetidin-1-yl}-5-cyano-2-{[(3,4-dimethoxybenzyl)oxy]methyl}nicotinate

A microwave vial was charged with ethyl 5-cyano-2-{[(3,4-dimethoxybenzyl)oxy]methyl}-6-[(methylsulfonyl)oxy]nicotinate (590 mg, 1.18 mmol), N-(benzylsulfonyl)azetidine-3-carboxamide (329 mg, 1.30 mmol), DIPEA (380 mg, 2.95 mmol) and EtOH (10 mL) and heated to 100° C. in a microwave oven for 10 minutes. Water (20 mL) was added and the water phase was made acidic with 1 M HCl and extracted with DCM (10 mL). The organic phase was separated and used in the next step as such.

### (c) Ethyl 6-{3-[(benzylsulfonyl)carbamoyl]azetidin-1-yl}-5-cyano-2-(hydroxymethyl)nicotinate

DDQ (133 mg, 0.585 mmol) was added to the DCM solution from above together with water (0.5 mL). The mixture was stirred at r.t for 15 minutes and passed through a phase separator to remove precipitated solids. ¼ of the crude product was subjected to purification by preparative HPLC (Kromasil C₈, 10 μm, using a gradient of 10-60% CH₃CN /0.1 M NH₄OAc, followed by a gradient of CH₃CN/0.2% HOAc in water (pH 4)) to give the desired product. Yield: 23.1 mg (4%, 20% calculated on that only ¼ of the crude was taken to purification)

¹H NMR (500 MHz, THF-d₈) δ 1.23 (4H, t, J=7.2 Hz), 3.40 (4H, quintet, J=7.7 Hz), 4.10 (1H, s), 4.18 (2H, q, J=7.1 Hz), 4.35-4.48 (4H, m), 4.57 (2H, s), 4.75 (2H, s), 7.22-7.29 (5H, m), 8.25 (1H, s), 10.41 (1H, s)

MS m/z: 459 (M+1), 457 (M−1)

GTPγS(IC₅₀ μM): 0.047

### Example 38

### Ethyl 6-{3-[(benzylsulfonyl)carbamoyl]azetidin-1-yl}-5-cyano-2-ethoxynicotinate

### (a) Ethyl 6-[3-(tert-butoxycarbonyl)azetidin-1-yl]-5-cyano-2-ethoxynicotinate

Ethyl iodide (449 mg, 2.88 mmol) was added to a mixture of ethyl 6-[3-(tert-butoxycarbonyl)azetidin-1-yl]-5-cyano-2-oxo-1,2-dihydropyridine-3-carboxylate (200 mg, 0.576 mmol) and Ag₂CO₃ (397 mg, 1.44 mmol) in dry CH₃CN (15 mL) and the mixture was heated to reflux over night. The reaction was filtered and the solvent evaporated to give the product which was used without further purification. Yield: 216 mg (99%).

MS m/z: 376 (M+1).

### (b) 1-[3-Cyano-6-ethoxy-5-(ethoxycarbonyl)pyridin-2-yl]azetidine-3-carboxylic acid

Tfa (1.77 ml, 23 mmol) was added to a solution of ethyl 6-[3-(tert-butoxycarbonyl)azetidin-1-yl]-5-cyano-2-ethoxynicotinate (216 mg, 0.575 mmol) in dcm (5 ml) and the mixture was stirred at r.t for 2 hours. The solvent and excess tfa was removed in vaccuo to give the crude product which was used without further purification. Crude yield: 645 mg (112%)

¹H NMR (400 MHz, DMSO-d₆) δ 1.25 (3H, t, J=7.3 Hz), 1.30 (3H, t, J=7.3 Hz), 4.16 (2H, q, J=7.3 Hz), 4.24-4.40 (4H, m), 4.42-4.53 (2H, m), 8.21 (1H, s)

MS m/z: 320 (M+1), 318 (M−1)

### (c) Ethyl 6-{3-[(benzylsulfonyl)carbamoyl]azetidin-1-yl}-5-cyano-2-ethoxynicotinate

1-[3-cyano-6-ethoxy-5-(ethoxycarbonyl)pyridin-2-yl]azetidine-3-carboxylie acid (32 mg, 0.10 mmol) dissolved in dcm (2 ml) and dipea (129.2 mg, 1 mmol) were added to a solution of 1-phenylmethanesulfonamide (18.8 mg, 0.11 mmol) and pybrop (70 mg, 0.15 mmol) in dcm (1 ml) and the mixture was stirred at r.t for 40 minutes. The organic phase was washed with 1% KHSO₄ (1 ml). The water phase was back extracted with dcm (0.5 ml) and the combined organic phase was passed through a phase separator and evaporated to give a crude product which was purified according purification method a (see general experimental procedure) to give the pure product.

¹H NMR (600 MHz, DMSO-d₆) δ 1.22-1.25 (3H, m), 1.30 (3H, t, J=7.1 Hz), 3.50-3.56 (1H, m), 4.15 (2H, q, J=7.1 Hz), 4.22-4.29 (2H, m), 4.33-4.41 (4H, m), 4.73 (2H, s), 7.30-7.37 (5H, m), 8.21 (1H, s)

MS m/z: 474 (M+1)

GTPγS(IC₅₀ μM): 0.047

### Example 39

### Ethyl 5-cyano-2-ethoxy-6-(3-{[(4-fluorobenzyl)sulfonyl]carbamoyl}azetidin-1-yl)nicotinate

Prepared according to the procedure described in Example 38(c) using 1-[3-Cyano-6-ethoxy-5-(ethoxycarbonyl)pyridin-2-yl]azetidine-3-carboxylic acid (32 mg, 0.10 mmol) and 1-(4-fluorophenyl)methanesulfonamide (20.8 mg, 0.11 mmol).

¹H NMR (600 MHz, DMSO-d₆) δ 1.23 (3H, t, J=7.2 Hz), 1.30 (3H, t, J=7.1 Hz), 3.50-3.56 (1H, m), 4.15 (2H, q, J=7.2 Hz), 4.21-4.30 (2H, m), 4.32-4.43 (2H, m), 4.36 (2H, q, J=7.1 Hz), 4.73 (2H, s), 7.17-7.21 (2H, m), 7.35-7.38 (2H, m), 8.20 (1H, s)

MS m/z: 491 (M+1).

GTPγS(IC₅₀ μM): 0.032

### Example 40

### Ethyl 5-cyano-2-ethoxy-6-(3-{[(2-fluorobenzyl)sulfonyl]carbamoyl}azetidin-1-yl)nicotinate

Prepared according to the procedure described in Example 38(c) using 1-[3-Cyano-6-ethoxy-5-(ethoxycarbonyl)pyridin-2-yl]azetidine-3-carboxylic acid (32 mg, 0.10 mmol) and 1-(2-fluorophenyl)methanesulfonamide (20.8 mg, 0.11 mmol).

¹H NMR (600 MHz, DMSO-d₆)δ 1.23 (3H, t, J=7.1 Hz), 1.30 (3H, t, J=6.8 Hz), 3.53-3.59 (1H, m), 4.15 (2H, q, J=7.1 Hz), 4.25-4.48 (6H, m), 4.78 (2H, s), 7.19-7.25 (2H, m), 7.41-7.46 (2H, m), 8.21 (1H, s)

MS m/z: 491 (M+1).

GTPγS(IC₅₀ μM): 0.031

### Example 41

### Ethyl 5-cyano-6-(3-{[(2,4-difluorobenzyl)sulfonyl]carbamoyl}azetidin-1-yl)-2-ethoxynicotinate

Prepared according to the procedure described in Example 38(c) using 1-[3-Cyano-6-ethoxy-5-(ethoxycarbonyl)pyridin-2-yl]azetidine-3-carboxylic acid (32 mg, 0.10 mmol) and 1-(2,4-difluorophenyl)methanesulfonamide (22.8 mg, 0.11 mmol).

¹H NMR (600 MHz, DMSO-d₆) δ 1.23 (3H, t, J=7.0 Hz), 1.29 (3H, t, J=7.4 Hz), 3.51-3.57 (1H, m), 4.15 (2H, q, J=7.0 Hz), 4.18-4.27 (2H, m), 4.30-4.41 (4H, m), 4.76 (2H, s), 7.16-7.20 (1H, m), 7.37-7.45 (2H, m), 8.20 (1H, s)

MS m/z: 509 (M+1).

GTPγS(IC₅₀ μM): 0.087

### Example 42

### Ethyl 6-{3-[(benzylsulfonyl)carbamoyl]azetidin-1-yl}-5-cyano-2-{[(3,4-dimethoxybenzyl)oxy]methyl}nicotinate

See Example 37(b).

GTPγS(IC₅₀ μM): 0.135

### Example 43

### Ethyl 5-chloro-6-(4-{[(4-chlorobenzyl)sulfonyl]carbamoyl}piperidin-1-yl)-2-(methylthio)nicotinate

### (a) Ethyl 2,6-dichloronicotinate

2,6-Dichloronicitinic acid (3.84 g, 20 mmol) was dissolved in EtOH (16 mL), sulfuric acid (1.96 g, 20 mmol) and triethyl ortoformate (4.45 g, 30 mmol) were added. The reaction mixture was heated in a microwave oven (single node heating) at 150° C. for 15 min. The mixture was extracted with EtOAc (3×20 mL) from 10% Na₂CO₃ (20 mL). The combined organic phases were extracted with water (50 mL), dried (Na₂SO₄), filtered and concentrated in vacuo to give ethyl 2,6-dichloronicotinate. The crude material was used in the next step without further purification.

### (b) Ethyl 6-[4-(tert-butoxycarbonyl)piperidin-1-yl]-2-chloronicotinate

Ethyl 2,6-dichloronicotinate (1.25 g, 5.68 mmol) was dissolved in DMF (16 mL), 4-piperidinecarboxylic acid tert-butyl ester hydrogen chloride (1.39 g, 6.25 mmol) and DIPEA (2.9 mL, 17 mmol) were added. The reaction mixture was heated in a microwave at 150° C. in a microwave oven (single node heating) for 10 min, the solvent was concentrated in vacuo and brine (8 mL) was added and the water phase was extracted with DCM (3×), the organic phase was dried (phase separator) and concentrated in vacuo. The residue was purified by flash chromatography, heptane/Et₂O 10:1 to 4:1 as eluent, to give ethyl 6-[4-(tert-butoxycarbonyl)piperidin-1-yl]-2-chloronicotinate. Yield: 630 mg (30%).

### (c) Ethyl 6-[4-(tert-butoxycarbonyl)piperidin-1-yl]-2,5-dichloronicotinate

Ethyl 6-[4-(tert-butoxycarbonyl)piperidin-1-yl]-2-chloronicotinate (621 mg, 1.68 mmol) was dissolved in acetonitrile (6 mL), N-chlorosuccinimide (292 mg, 2.2 mmol) was added and the reaction mixture was heated in a microwave oven (single node heating) at 100° C. for 10 min. The solvent was concentrated in vacuo and the residue was purified by flash chromatography, heptane/Et₂O 6:1 to 4:1 as eluent, to give ethyl 6-[4-(tert-butoxycarbonyl)piperidin-1-yl]-2,5-dichloronicotinate as an oil. Yield: 560 mg (83%).

¹H NMR (400 MHz, CDCl₃) δ 1.38 (3H, t), 1.46 (9H, s), 1.74-1.89 (2H, m), 1.94-2.04 (2H, m), 2.43-2.52 (1H, m), 3.02-3.13 (2H, m), 4.07-4.16 (2H, m), 4.35 (2H, q), 8.07 (1H, s).

MS m/z: 403 (M+1)

### (d) Ethyl 6-[4-(tert-butoxycarbonyl)piperidin-1-yl]-5-chloro-2-(methylthio)nicotinate

Sodium thiomethylate (26 mg, 0.375 mmol) was added to a solution of ethyl 6-[4-(tert-butoxycarbonyl)piperidin-1-yl]-2,5-dichloronicotinate (101 mg (0.250 mmol) in DMSO (3 mL) in a microwave vial and the mixture was heated to 80° C. for 5 min. The crude product was purified by preparative HPLC (Kromasil C₈, 10 mm, using a gradient 5% to 100% MeCN with an acidic second eluent (H₂O/MeCN/AcOH, 95/5/0.1) to give ethyl 6-[4-(tert-butoxycarbonyl)piperidin-1-yl]-5-chloro-2-(methylthio)nicotinate. Yield: 85 mg (82%).

MS m/z: 415 (M+1)

### (e) 1-[3-Chloro-5-(ethoxycarbonyl)-6-(methylthio)pyridin-2-yl]piperidine-4-carboxylic acid

Ethyl 6-[4-(tert-butoxycarbonyl)piperidin-1-yl]-5-chloro-2-(methylthio)nicotinate was dissolved in DCM/TFA (1/1, 1 mL) and stirred at r.t for 2.5 hours. The solvent and excess TFA was removed in vaccuo to give 1-[3-Chloro-5-(ethoxycarbonyl)-6-(methylthio)pyridin-2-yl]piperidine-4-carboxylic acid which was used without further purification. Yield: 73 mg (99%).

¹H NMR (400 MHz, CDCl₃): δ 1.38 (3H, t, J=7.1 Hz), 1.86-1.98 (2H, m), 2.03-2.12 (2H, m), 2.48 (3H, s), 2.60-2.70 (1H, m), 3.08-3.17 (2H, m), 4.14-4.23 (2H, m), 4.35 (2H, q, J=7.1 Hz), 8.07 (1H, s), 10.66 (1H, s).

MS m/z: 359 (M+1)

### (f) Ethyl 5-chloro-6-(4-{[(4-chlorobenzyl)sulfonyl]carbamoyl}piperidin-1-yl)-2-(methylthio)nicotinate

DIPEA (133 mg, 1.03 mmol) was added to a mixture of 1-[3-chloro-5-(ethoxycarbonyl)-6-(methylthio)pyridin-2-yl]piperidine-4-carboxylic acid (37 mg, 0.103 mmol), 1-(4-chlorophenyl)methanesulfonamide (24 mg, 0.118 mmol) and bromo(tripyrrolidin-1-yl)phosphonium hexafluorophosphate (72 mg, 0.155 mmol) in DCM (2 mL) and the mixture was stirred at r.t for 16 hours. The solvent was evaporated and the crude product was purified by Purification method A (See General Experimental Procedures) to give ethyl 5-chloro-6-(4-{[(4-chlorobenzyl)sulfonyl]carbamoyl}piperidin-1-yl)-2-(methylthio)nicotinate. Yield: 27 mg (49%).

¹H NMR (400 MHz, CDCl₃): δ 1.38 (3H, t, J=7.1 Hz),1.84-1.93 (4H, m), 2.40-2.50 (1H, m), 2.48 (3H, s), 2.93-3.02 (2H, m), 4.19-4.26 (2H, m), 4.34 (2H, q, J=7.1 Hz), 4.63 (2H, s), 7.26-7.32 (3H, m), 7.34-7.38 (2H, m), 8.07 (1H, m), 9.52 (1H, s).

MS m/z: 546 (M+1)

GTPγS(IC₅₀ μM): 0.132

### Example 44

### Ethyl 6-{4-[(benzylsulfonyl)carbamoyl]piperidin-1-yl}-5-fluoro-2-(methylthio)nicotinate

### (a) 2,6-Dichloro-5-fluoronicotinoyl chloride

A suspension of 2,6-dichloro-5-fluoronicotinic acid (4.3 g, 20.5 mmol) in toluene (20 mL) and thionyl chloride (20 mL, 276 mmol) was refluxed under an N₂-atmosphere for 3 hours. The mixture was cooled and the solvent was concentrated in vacuo and the residue was co-evaporated twice with toluene to give 2,6-dichloro-5-fluoronicotinoyl chloride as a yellow oil which was used in the next step without further purification assuming quantitative yield of the product.

### (b) Ethyl 2,6-dichloro-5-fluoronicotinate

Cold ethanol (40 mL) was added to 2,6-dichloro-5-fluoronicotinoyl chloride (4.7 g, 20.5 mmol)) at 0° C., the mixture was stirred for 15 minutes at 0° C. followed by 1 hour at reflux under an N₂-atmosphere. The EtOH was concentrated in vacuo and the residue was dissolved in EtOAc (130 mL) and the organic phase was washed with KHCO₃ (15 mL), water (15 mL), brine (15 mL) and dried (MgSO₄) and concentrated in vacuo to give ethyl 2,6-dichloro-5-fluoronicotinate as oil. The crude product was used in the next step without further purification. Yield: 4.64 g (95%).

¹H NMR (400 MHz, CDCl₃) δ 1.42 (3H, t), 4.44 (2H, q), 8.00 (1H, d).

### (c) ethyl 6-{4-[(benzylsulfonyl)carbamoyl]piperidin-1-yl}-2-chloro-5-fluoronicotinate

DIPEA (1.293 g, 10 mmol) was added to a slurry of ethyl 2,6-dichloro-5-fluoronicotinate (1.19 g, 5 mmol) and N-(benzylsulfonyl)piperidine-4-carboxamide (1.412 g, 5 mmol) in EtOH and the mixture was heated to 90° C. (N₂-atmosphere) over night (19 hours) to give a yellow solution. The solvent was evaporated and the product was taken up in EtOAc (150 mL) and washed with NH₄Cl (2×15 mL), water (1×15 mL), Brine (15 mL). The organic phase was dried (Na₂SO₄), filtered and evaporated to give 2.35 g of a white foamy solid. The solvents were evaporated off and EtOH (99.5%, 25 mL) was added and the slurry was stirred at 60 deg for 2 hours. The solid was filtered off after cooling to room temperature and washed with EtOH (5 mL) and dried in vaccuo to give the pure product as a white solid. Yield: 1.89 g (78%).

¹H NMR (400 MHz, DMSO-d₆) δ 1.29 (3H, t), 1.55-1.68 (2H, m), 1.75-1.84 (2H, m), 2.5-2.59 (1H, m), 2.98-3.09 (2H, m), 4.20-4.30 (4H, m), 4.68 (2H, s), 7.25-7.30 /2H, m), 7.35-7.44 (3H, m), 7.89 (1H, d), 11.57 (1H, s).

MS m/z: 484 (M+1), 482 (M−1).

### (d) Ethyl 6-{4-[(benzylsulfonyl)carbamoyl]piperidin-1-yl}-5-fluoro-2-(methylthio)nicotinate

NaSMe (40.9 mg, 0.58 mmol) was added to a solution of ethyl 6-{4-[(benzylsulfonyl)carbamoyl]piperidin-1-yl}-2-chloro-5-fluoronicotinate (114 mg, 0.24 mmol) in NMP (3 mL), and the reaction mixture was stirred at r.t for two days. LCMS showed product and starting material in a ratio 27:53. The reaction mixture was therefore heated to 100° C. for 5 min in a single node microwave oven. LCMS showed product and starting material in a ratio 27:50. More NaSMe (40 mg, 0.57 mmol) was added and the reaction mixture was heated to 120° C. for 10 min in a single node microwave oven. LCMS showed product and starting material in a ratio 58:20. Additional NaSMe (40 mg, 0.57 mmol) was added and the reaction mixture was heated to 120° C. for 10 min in a single node microwave oven. LCMS showed complete conversion of the starting material. NaHCO₃(aq) was added and the mixture was extracted with DCM(×3). The combined organic phase was run through a phase separator and evaporated. The crude product was purified by Purification Method A (See General Experimental Procedures)) to give ethyl 6-{4-[(benzylsulfonyl)carbamoyl]piperidin-1-yl}-5-fluoro-2-(methylthio)nicotinate as a solid. Yield: 53.1 mg (43%).

MS m/z: 496 (M+1), 494 (M−1).

GTPγS(IC₅₀ μM): 0.042

### Example 45

### Ethyl 6-{4-[(benzylsulfonyl)carbamoyl]piperidin-1-yl}-5-cyano-2-(2-methoxyethyl)nicotinate

### (a) Ethyl 5-cyano-2-(2-methoxyethyl)-6-oxo-1,6-dihydropyridine-3-carboxylate

Malonitrile (2.043 g, 30.93 mmol) dissolved in EtOH (15 mL) was added during 3 minutes to a solution of ethyl 2-[(dimethylamino)methylene]-5-methoxy-3-oxopentanoate (6.45 g, 28.12 mmol) and TEA (0.285 g, 2.81 mmol) in EtOH (10 mL) (slightly exotermic reaction) under an atmosphere of nitrogen. The mixture was stirred for 26 hours at r.t and HOAc (1.93 mL) was added dropwise to give a precipitate. The mixture was heated to 70 degrees (homogenous solution) and water (45 mL) was added to give a precipitate. The mixture was placed in the refrigerator over night and the solid was filtered off and washed with cold water (3×20 mL). The solid was dried in vaccuo to give the product as a yellow-green powder. Yield: 4.97 g (70%),

¹H NMR (400 MHz, DMSO-d₆) δ 1.27 (3H, t), 3.21 (3H, s), 3.24 (2H, t), 3.56 (2H, t), 4.22 (2H, q), 8.45 (1H, s), 12.95 (1H, bs)

MS m/z: 251 (M+1), 249 (M−1)

### (b) Ethyl 6-chloro-5-cyano-2-(2-methoxyethyl)nicotinate

A slurry of ethyl 5-cyano-2-(2-methoxyethyl)-6-oxo-1,6-dihydropyridine-3-carboxylate (4.95 g, 19.78 mmol) and POCl₃ (4.85 g, 31.65 mmol) in CH₃CN (30 mL) was heated to 80 degrees under an atmosphere of nitrogen (the slurry became a homogenoues dark green solution after about 10-15 minutes) for 26 hours (dark red-brown solution). MTBE (methyl-tertbuthyleher, 90 mL) was added and the mixture was cooled down in an ice/water bath followed by addition of water (30 mL). The phases were separated and the water phase was extracted with 50 mL MTBE. the combined organic phase was washed with water (20 mL), 5% K₂CO₃ (aq) (2×20 mL). Evaporation of the solvent gave 5.48 g of a red oil. The crude was dissolved in EtOAc and washed with 1×10 mL Brine to give 4.58 g of a red oil. This was subjected to chromatography using the Biotage system (Eluent 0-30% Heaxane/EtOAc, 1 column volume out followed by 10 column volumes) This gave the product as a solid. Yield: 0.5 g (9%).

¹H NMR (400 MHz, CDCl₃): δ1.40 (3H, t), 3.32 (3H, s), 3.54 (2H, t), 3.79 (2H, t), 4.42 (2H, q), 8.44 (1H, s)

MS m/z: 269 (M+1), 267 (M−1)

### (c) Ethyl 6-{4-[(benzylsulfonyl)carbamoyl]piperidin-1-yl}-5-cyano-2-(2-methoxyethyl)nicotinate

Ethyl 6-chloro-5-cyano-2-(2-methoxyethyl)nicotinate (100 mg, 0.372 mmol), N-(benzylsulfonyl)piperidine-4-carboxamide (105 mg, 0.372 mmol), DIPEA (96 mg, 0.744 mmol) and EtOH (3 mL) was charged to a microwave vial and heated in a single node microwave oven for 10 minutes. LC-MS showed complete conversion. The solvent was removed and the crude product was purified by preparative HPLC(Kromasil C₈, 10 μM, 250×20 mm ID, Mobilephase A (water/acetonitrile/HCOOH 95/5/0.2), B (CH3CN) (A continous gradient of A/B, from 65/35 to 40/60 for 20 minutes was used and the compound Eluted at A/B ratio of 40/60). the relevant fractions was collected, evaporated and freeze dried to give the pure product as a white solid. Yield: 130 mg (68%).

1H-NMR (DMSO-d₆): δ 1.29 (3H, t, J=7.0 Hz), 1.57-1.59 (2H, m), 1.79-1.87 (2H, m), 2.54-2.63 (1H, m), 3.14 (2H, apparent t), 3.20 (3H, s), 3.29 (2H, t, J=6.7 Hz), 3.69 (2H, t, J=6.7 Hz), 4.25 (2H, q, J=7.0 Hz), 4.53 (2H, apparent d), 4.69 (2H, s), 7.26-7.31 (2H, m), 7.37-7.42 (3H, m), 8.33 (1H, s), 11.60 (1H, bs, NH).

MS m/z: 515 (M+1), 513 (M−1)

GTPγS(IC₅₀ μM): 0.06

### Example 46

### Ethyl 6-{4-[(benzylsulfonyl)carbamoyl]piperidin-1-yl}-2-chloro-5-fluoronicotinate

See Example 44(c).

GTPγS(IC₅₀ μM): 0.048

### Example 47

### Ethyl 6-{4-[(benzylsulfonyl)carbamoyl]piperidin-1-yl}-5-cyano-2-(1H-1,2,4-triazol-1-ylmethyl)nicotinate

A microwave vial was charged with ethyl 6-chloro-2-(chloromethyl)-5-cyanonicotinate (Example 34(a)) (50 mg, 0.099 mmol), 1,2,4-triazole (27 mg, 0.396 mmol), NaI (1.5 mg, 0.01 mmol) and EtOH (1mL) and heated to 100° C. for 15 minutes using a microwave oven. The solvent was evaporated and the crude product was purified by preparative HPLC (Kromasil C₈, 10 mm, using a gradient of MeCN with an acidic second eluent (H₂O/MeCN/FA, 95/5/0.2)) to give ethyl 6-{4-[(benzylsulfonyl)carbamoyl]piperidin-1-yl}-5-cyano-2-(1H-1,2,4-triazol-1-ylmethyl)nicotinate. Yield: 12 mg (22%).

¹H NMR (400 MHz, CDCl₃) δ 1.21 (3H, t, J=7.2 Hz), 1.40-1.53 (2H, m), 1.57-1.66 (2H, m), 2.80-2.92 (2H, m), 4.10-4.21 (4H, m), 4.39 (2H, s), 5.56 (2H, s), 7.08-7.15 (2H, m), 7.17-7.28 (3H, m), 8.21 (1H, s)

MS m/z: 538 (M+1), 536 (M−1)

GTPγS(IC₅₀ μM): 0.077

### Example 48

### Ethyl 6-{4-[(benzylsulfonyl)carbamoyl]piperidin-1-yl}-5-cyano-2-(1H-1,2,3-triazol-1-ylmethyl)nicotinate

A microwave vial was charged with ethyl 6-chloro-2-(chloromethyl)-5-cyanonicotinate (Example 34(a)) (50 mg, 0.099 mmol), 1,2,3-triazole (27 mg, 0.396 mmol), NaI (1.5 mg, 0.01 mmol) and EtOH (1 mL) and heated to 100° C. for 15 minutes using a microwave oven. The solvent was evaporated and the crude product was purified by preparative HPLC (Kromasil C₈, 10 mm, using a gradient of MeCN with an acidic second eluent (H₂O/MeCN/FA, 95/5/0.2)) to give ethyl 6-{4-[(benzylsulfonyl)carbamoyl]piperidin-1-yl}-5-cyano-2-(1H-1,2,3-triazol-1-ylmethyl)nicotinate. Yield: 23 mg (43%).

¹H NMR (400 MHz, CDCl₃) δ 1.40 (3H, t, J=7.0 Hz), 1.51-1.64 (2H, m), 1.67-1.80 (2H, m), 2.59-2.69 (1H, m), 3.02 (2H, t, J=11.9 Hz), 4.20 (2H, d, J=13.7 Hz), 4.36 (2H, q, J=7.1 Hz), 4.60 (2H, s), 6.05 (2H, s), 7.27-7.40 (5H, m), 7.61-7.77 (2H, br m), 8.39 (1H, s), 9.61 (1H, s)

MS m/z: 538 (M+1), 536 (M−1)

GTPγS(IC₅₀ μM): 0.032

### Example 49

### Ethyl 6-{4-[(benzylsulfonyl)carbamoyl]piperidin-1-yl}-5-cyano-2-(1H-imidazol-1-ylmethyl)nicotinate

A microwave vial was charged with ethyl 6-chloro-2-(chloromethyl)-5-cyanonicotinate (Example 34(a)) (50 mg, 0.099 mmol), imidazole (27 mg, 0.396 mmol), NaI (1.5 mg, 0.01 mmol) and EtOH (1 mL) and heated to 100° C. for 15 minutes using a microwave oven. The solvent was evaporated and the crude product was purified by preparative HPLC (Kromasil C₈, 10 mm, using a gradient of MeCN with an acidic second eluent (H₂O/MeCN/FA, 95/5/0.2)) to give ethyl 6-{4-[(benzylsulfonyl)carbamoyl]piperidin-1-yl}-5-cyano-2-(1H-imidazol-1-ylmethyl)nicotinate. Yield: 17 mg (32%).

¹H NMR (400 MHz, CDCl₃) δ 1.31 (3H, t, J=7.1 Hz), 1.44-1.57 (2H, m), 1.66-1.74 (2H, m), 2.98 (2H, br t, J=11.5 Hz), 4.19 (2H, br d, J=13.5 Hz), 4.28 (2H, q, J=7.2 Hz), 4.50 (2H, s), 5.85 (2H, s), 7.18-7.23 (2H, m), 7.26-7.32 (3H, m), 7.40 (1H, t, J=1.5 Hz), 7.44 (1H, t, J=1.6 Hz), 8.33 (1H, s), 8.97 (1H, s)

MS m/z: 537 (M+1), 535 (M−1)

GTPγS(IC₅₀ μM): 0.073

### Example 50

### Isopropyl 6-{4-[(benzylsulfonyl)carbamoyl]piperidin-1-yl}-2,5-dicyanonicotinate

### (a) Isopropyl 6-[4-(tert-butoxycarbonyl)piperidin-1-yl]-5-cyano-2-oxo-1,2-dihydropyridine-3-carboxylate

Prepared in essentially the same way as described in Example 7(b) from Diisopropyl(ethoxymethylene)malonate (5.54g, 22.7 mmol) and tert-Butyl 1-(2-cyanoethanimidoyl)piperidine-4-carboxylate (3.80 g, 15.12 mmol) to give isopropyl 6-[4-(tert-butoxycarbonyl)piperidin-1-yl]-5-cyano-2-oxo-1,2-dihydropyridine-3-carboxylate. Yield: 2.44 g (41%).

### (b) Isopropyl 6-[4-(tert-butoxycarbonyl)piperidin-1-yl]-5-cyano-2-{[(trifluoromethyl)sulfonyl]oxy}nicotinate

(Tf)₂O (797 mg, 2.82 mmol) was added during 5 minutes to a cold solution (ice/water bath temperature) of isopropyl 6-[4-(tert-butoxycarbonyl)piperidin-1-yl]-5-cyano-2-oxo-1,2-dihydropyridine-3-carboxylate (1.00 g, 2.57 mmol) and TEA (779 mg, 7.7 mmol) in DCM (20 mL) and the mixture was stirred for 25 minutes. NaHCO₃ (aq) (20 mL) was added and the organic phase was separated, dried (MgSO₄), filtered and evaporated to give isopropyl 6-[4-(tert-butoxycarbonyl)piperidin-1-yl]-5-cyano-2-{[(trifluoromethyl)sulfonyl]oxy}nicotinate which was used without further purification in the next step. Yield: 1.49 g (111%, crude yield)

¹H-NMR (500 MHz, CDCl₃): δ 1.35 (6H, d), 1.45 (9H, s), 1.83 (2H, m), 2.04 (2H, m), 2.57 (1H, septett), 3.41 (2H, m), 4.50 (2H, m), 5.25 (1H, m), 8.50 (1H, s).

### (c) Isopropyl 6-[4-(tert-butoxycarbonyl)piperidin-1-yl]-2,5-dicyanonicotinate

A microwave vial was charged with isopropyl 6-[4-(tert-butoxycarbonyl)piperidin-1-yl]-5-cyano-2-{[(trifluoromethyl)sulfonyl]oxy}nicotinate (200 mg, 0.384 mmol), Pd₂(dba)₃ (53 mg, 0.058 mmol), Xantphos(33 mg, 0.058 mmol), sodium cyanide (56 mg, 1.15 mmol), DIPEA (0.2 mL, 1.15mmol) and dioxane(5mL) and the reaction mixture was heated to 160° C. for 20 minutes using microwave single node heating.

The mixture was filtered and diluted with diethyl ether. The organic phase was washed with water, dried (MgSO₄), filtered and evaporated to give 200 mg of a crude product as a syrup. The crude product was purified by flash chromatography using an increasing gradient of EtOAc in heptane (5 to 50%) to give isopropyl 6-[4-(tert-butoxycarbonyl)piperidin-1-yl]-2,5-dicyanonicotinate. Yield: 19 mg (12%).

¹H-NMR (500 MHz, CDCl₃): 1.40 (6H, d), 1.45 (9H, s), 1.81 (2H, m), 2.04 (2H, m), 2.57 (1H, septett), 3.39 (2H, m), 4.54 (2H, m), 5.28 (1H, m), 8.41 (1H, s).

### (d) 1-[3,6-Dicyano-5-(isopropoxycarbonyl)pyridin-2-yl]piperidine-4-carboxylic acid

TFA (1 mL) was added to a solution of isopropyl 6-[4-(tert-butoxycarbonyl)piperidin-1-yl]-2,5-dicyanonicotinate (19 mg, 0.047 mmol) in CHCl₃ and the mixture was stirred at r.t. for 1.5 hours. The solvent was evaporated to give 1-[3,6-Dicyano-5-(isopropoxycarbonyl)pyridin-2-yl]piperidine-4-carboxylic acid which was used without further purification in the next step. Yield: 16 mg (98%).

### (e) Isopropyl 6-{4-[(benzylsulfonyl)carbamoyl]piperidin-1-yl}-2,5-dicyanonicotinate

1-phenylmethanesulfonamide (10 mg, 0.047 mmol) was added to a solution of 1-[3,6-dicyano-5-(isopropoxycarbonyl)pyridin-2-yl]piperidine-4-carboxylic acid (16 mg, 0.047 mmol), DIPEA (62 mg, 0.477 mmol) and PyBrop (33 mg, 0.072 mmol) in DCM (1 mL) at r.t. The mixture was stirred over night, diluted with DCM and extracted with water. The solvent was dried, filtered and evaporated to give a crude product which was purified by preparative HPLC (Kromasil C₈, 10 mm, using a gradient of MeCN with an acidic second eluent (H₂O/MeCN/FA, 95/5/0.2)) to give isopropyl 6-{4-[(benzylsulfonyl)carbamoyl]piperidin-1-yl}-2,5-dicyanonicotinate. Yield: 1 mg (4%).

¹H-NMR (500 MHz, CDCl₃): 1.41 (6H, d), 1.75-1.95 (4H, m), 2.46 (1H, septett), 3.26 (2H, m), 4.65 (2H, m) 4.67, (2H, s), 5.29 (1H, m), 7.30-7.45 (5H, m), 8.45 (1H, s).

GTPγS(IC₅₀ μM): 0.016

### Example 51

### 1-(5-Butyryl-3-cyano-6-methoxypyridin-2-yl)-N-[(4-fluorobenzyl)sulfonyl]piperidine-4-carboxamide

### (a) 6-[4-(tert-butoxycarbonyl)piperidin-1-yl]-5-cyano-2-methoxynicotinic acid

A microwave vial was charged with NaOH (0.40 g, 10 mmol), ethyl 6-[4-(tert-butoxycarbonyl)piperidin-1-yl]-5-cyano-2-methoxynicotinate (389 mg, 1 mmol) and MeCN/water (1/1, 8 mL) and the mixture was heated to 80° C. for 5 minutes using microwave single node heating. FA (1 mL) was added and the mixture was extracted with DCM (3×5 mL). The solvent was evaporated to give 6-[4-(tert-butoxycarbonyl)piperidin-1-yl]-5-cyano-2-methoxynicotinic acid which was used in the next step without further purification. Yield: 395 mg (109%, crude).

### (b) tert-Butyl 1-{3-cyano-6-methoxy-5-[methoxy(methyl)carbamoyl]pyridin-2-yl}piperidine-4-carboxylate

DIPEA (1.32 g, 10.24 mmol) was added to a solution of 6-[4-(tert-butoxycarbonyl)piperidin-1-yl]-5-cyano-2-methoxynicotinic acid (370 mg, 1.02 mmol), N,O-dimetylhydroxylamine hydrochloride (300 mg, 3.07 mmol) and PyBrop (716 mg, 1.54 mmol) in DCM (10 mL) and the mixture was stirred at r.t. for 3 hours. The mixture was washed with water (5 mL), dried and evaporated to give a crude product which was purified by preparative HPLC (Kromasil C₈, 10 mm, using a gradient of MeCN with an acidic second eluent (H₂O/MeCN/HOAc, 95/5/0.1)) to give tert-butyl 1-{3-cyano-6-methoxy-5-[methoxy(methyl)carbamoyl]pyridin-2-yl}piperidine-4-carboxylate. Yield: 734 mg (72%).

### (c) tert-Butyl 1-(5-butyryl-3-cyano-6-methoxypyridin-2-yl)piperidine-4-carboxylate

n-PrMgCl (0.76 mL 2 M solution in Et₂O, 2 eq) was added to a cold (−78° C.) solution of tert-butyl 1-{3-cyano-6-methoxy-5-[methoxy(methyl)carbamoyl]pyridin-2-yl}piperidine-4-carboxylate (307 mg, 0.759 mmol) in THF (10 mL) under an atmosphere of nitrogen. The reaction was stirred at −78° C. for 30 minutes followed by r.t. for 20 minutes. An aliquot was taken out and quenched with water and then dissolved in DMSO/methanol 1:1. LC/MS showed that no A had been converted.

The reaction mix was therefore cooled again to −78 degr. and further n-PrMgCl (3.8 mL 2 M solution in Et₂O, 10 eq) was added. After 10 min. the cooling bath was removed and the reaction mix allowed to reach r.t during 1 h. LC/MS on an aliquot treated as above showed full conversion to the product. Water (5 mL) was added and the mix was extracted with DCM (3×5 mL) by using a phase separator and the combined organic phase was evapoarted to give tert-butyl 1-(5-butyryl-3-cyano-6-methoxypyridin-2-yl)piperidine-4-carboxylate which was used without further purification in the next step.

¹H NMR (400 MHz, CDCl₃): δ 0.96 (3H, t, J=7.4 Hz), 1.45 (9H, s), 1.62-1.73 (2H, m), 1.78-1.84 (2H, m), 1.96-2.06 (2H, m), 2.50-2.59 (1H, m), 2.86 (2H, t, J=7.3 Hz), 3.27-3.36 (2H, m), 4.00 (3H, s), 4.52-4.60 (2H, m), 8.33 (1H, s).

MS m/Z: 388 (M+1)

### (d) 1-(5-Butyryl-3-cyano-6-methoxypyridin-2-yl)piperidine-4-carboxylic acid

A solution of tert-butyl 1-(5-butyryl-3-cyano-6-methoxypyridin-2-yl)piperidine-4-carboxylate (10 mg, 0.026 mmol) in DCM/TFA (1/1, 1 mL) was stirred at r.t. for 2.5 hours. The solvent and excess TFA was evaporated to give 1-(5-butyryl-3-cyano-6-methoxypyridin-2-yl)piperidine-4-carboxylic acid which was used without further purification.

MS m/z: 330 (M−1)

### (e) 1-(5-Butyryl-3-cyano-6-methoxypyridin-2-yl)-N-[(4-fluorobenzyl)sulfonyl]piperidine-4-carboxamide

DIPEA (211 mg, 1.63 mmol) was added to a solution of 1-(4-fluorophenyl)methanesulfonamide (46 mg, 0.25 mmol), PyBrop (114 mg, 0.245 mmol) and 1-(5-butyryl-3-cyano-6-methoxypyridin-2-yl)piperidine-4-carboxylic acid (54 mg, 0.163 mmol) in DCM (2 mL) and the mixture was stirred at r.t. for 22 hours.

Water (1 mL) was added. The organic phase was separated and the aq. phase extracted with DCM (2×1 mL) by using a phase separator. The organic phases were combined and concentrated and the crude material was purified by Purification Method A (See General Experimental Procedure) to give 1-(5-butyryl-3-cyano-6-methoxypyridin-2-yl)-N-[(4-fluorobenzyl)sulfonyl]piperidine-4-carboxamide. Yield: 42 mg (51%).

¹H-NMR (600 MHz, DMSO-d₆) δ 0.87 (3H, t, J=7.4 Hz), 1.51-1.58 (2H, m), 1.59-1.67 (2H, m), 1.81-1.86 (2H, m), 2.50-2.56 (1H, m, hidden under DMSO signal), 2.83 (2H, t, J=7.2 Hz), 3.14-3.20 (2H, m), 3.96 (3H, s), 4.53-4.58 (2H, m), 4.69 (2H, s), 7.20-7.25 (2H, m), 7.29-7.34 (2H, m), 8.23 (1H, s), 11.60 (1H, s).

MS m/z: 503 (M+1)

GTPγS(IC₅₀ μM): 0.05

### Example 52

### 1-(5-Butyryl-3-cyano-6-methoxypyridin-2-yl)-N-[(4(4-chlorobenzyl)sulfonyl]piperidine-4-carboxamide

Prepared according to the procedure described in Example 51(e) using 1-(4-chlorophenyl)methanesulfonamide (50 mg, 0.245 mmol) and 1-(5-butyryl-3-cyano-6-methoxypyridin-2-yl)piperidine-4-carboxylic acid (54 mg, 0.163 mmol). Yield: 40 mg (47%).

¹H-NMR (600 MHz, DMSO-d₆) δ 0.87 (3H, t, J=7.4 Hz), 1.51-1.58 (2H, m), 1.59-1.67 (2H, m), 1.81-1.86 (2H, m), 2.50-2.56 (1H, m, hidden under DMSO signal), 2.83 (2H, t, J=7.2 Hz), 3.14-3.21 (2H, m), 3.97 (3H, s), 4.53-4.58 (2H, m), 4.70 (2H, s), 7.28-7.31 (2H, m), 7.45-7.48 (2H, m), 8.23 (1H, s), 11.62 (1H, s).

MS m/z: 519 (M+1)

GTPγS(IC₅₀ μM): 0.055

### Example 53

### N-(Benzylsulfonyl)-1-(5-butyryl-3-cyano-6-methoxypyridin-2-yl)piperidine-4-carboxamide

Prepared according to the procedure described in Example 51(e) using 1-phenylmethanesulfonamide (42 mg, 0.245 mmol) and 1-(5-butyryl-3-cyano-6-methoxypyridin-2-yl)piperidine-4-carboxylic acid (54 mg, 0.163 mmol). Yield: 10 mg (12%).

¹H NMR (500 MHz, CDCl₃): δ 0.96 (3H, t, J=7.4 Hz), 1.62-1.72 (2H, m), 1.75-1.93 (4H, m), 2.42-2.51 (1H, m), 2.87 (2H, t, J=7.4 Hz), 3.13-3.22 (2H, m), 4.01 (3H, s), 4.61-4.69 (4H, m), 7.31-7.35 (2H, m), 7.36-7.42 (3H, m), 8.32 (1H, s).

MS m/z: 485 (M+1)

GTPγS(IC₅₀ μM): 0.076

### Example 54

### Ethyl 6-{4-[(benzylsulfonyl)carbamoyl]piperidin-1-yl}-5-chloro-2-(methylthio)nicotinate

Prepared according to the procedure described in Example 51(e) using 1-phenylmethanesulfonamide (20 mg, 0.118 mmol) and 1-[3-Chloro-5-(ethoxycarbonyl)-6-(methylthio)pyridin-2-yl]piperidine-4-carboxylic acid (37 mg, 0.103 mmol). Yield: 7 mg (13%).

¹H NMR (400 MHz, DMSO-d₆): δ 1.32 (3H, t, J=7.1 Hz),1.65-1.77 (2H, m), 1.78-1.86 (2H, m), 2.45 (3H, m), 2.50-2.56 (1H, m, hidden under DMSO signal), 2.94-3.05 (2H, m), 4.18-4.26 (2H, m), 4.27 (2H, q, J=7.1 Hz), 4.71 (2H, s), 7.29-7.34 (2H, m), 7.38-7.44 (3H, m), 8.04 (1H, s), 11.61 (1H, s).

MS m/z: 512 (M+1)

GTPγS(IC₅₀ μM): 0.039

### Example 55

### Isopropyl 6-(4-{[(4-chlorobenzyl)sulfonyl]carbamoyl}piperidin-1-yl)-5-cyano-2-methoxynicotinate

### (a) isopropyl 6-[4-(tert-butoxycarbonyl)piperidin-1-yl]-5-cyano-2-methoxynicotinate

Methyl iodide (200 mg, 1.41 mmol) and K₂CO₃ (195 mg, 1.41 mmol) was added to a solution of isopropyl 6-[4-(tert-butoxycarbonyl)piperidin-1-yl]-5-cyano-2-oxo-1,2-dihydropyridine-3-carboxylate (500 mg, 1 mmol) in DMF (8 mL) and the reaction mixture was stirred at r.t. for 16 hours. LC-MS indicated some remaining starting material and an addition small amount of methyl iodide and K₂CO₃ was added and the mixture was stirred for an additional 4 hours. DCM was added and the organic phase was washed with NaHCO₃(aq), dried and evaporated. Some DMF was still remaining after the extraction and the mixture was redissolved in MTBE (20 mL) and extracted with water (3×10 mL). The organic phase was dried (MgSO₄), filtered and evaporated to give isopropyl 6-[4-(tert-butoxycarbonyl)piperidin-1-yl]-5-cyano-2-methoxynicotinate which was used without further purification. Yield: 490 mg (95%).

¹H-NMR (CDCl₃): δ 1.27 (6H, d), 1.40 (9H, s), 1.75 (2H, m), 1.95 (2H, m), 2.50 (1H, septett), 3.26 (2H, m), 3.93 (3H, s), 4.50 (2H, m), 5.10 (1H, m), 8.23 (1H, s).

### (b) 1-[3-Cyano-5-(isopropoxycarbonyl)-6-methoxypyridin-2-yl]piperidine-4-carboxylic acid

A solution of isopropyl 6-[4-(tert-butoxycarbonyl)piperidin-1-yl]-5-cyano-2-methoxynicotinate (490 mg, 1.28 mmol) in DCM/TFA (2/1, 6 mL) and the mixture was stirred for 2.5 hours at r.t. The solvent and excess TFA was evaporated in vaccuo to give 1-[3-Cyano-5-(isopropoxycarbonyl)-6-methoxypyridin-2-yl]piperidine-4-carboxylic acid as a white solid in quantitative yield.

¹H-NMR (500 MHz, CD₃OD): δ 1.33 (6H, d), 1.79, (2H, m), 2.06 (2H, m), 2.70 (1H, septett), 3.36 (2H, m), 3.98 (3H, s), 4.60 (2H, m), 5.13 (1H, m), 8.27 (1H, s).

### (c) Isopropyl 6-(4-{[(4-chlorobenzyl)sulfonyl]carbamoyl}piperidin-1-yl)-5-cyano-2-methoxynicotinate

1-(4-chlorophenyl)methanesulfonamide (62 mg, 0.302 mmol) was added to a prestirred (1 hour) solution of 1-[3-cyano-5-(isopropoxycarbonyl)-6-methoxypyridin-2-yl]piperidine-4-carboxylic acid (100 mg, 0.288 mmol), TBTU (129 mg, 0.403 mmol) and DIPEA (74 mg, 0.576 mmol) in DCM (4 mL) and the mixture was stirred at r.t. over night. Water (2 mL), and NaHCO₃ (aq,sat) (2 mL) was added and the mixture was passed through a phase separator. The organic solvent was evapoarted to give 240 mg of a crude product which was first purified by preparative HPLC (Kromasil C₈, 10 mm, using a gradient of increasing MeCN with a second eluent (0.1 M NH₄OAc/MeCN, 95/5)) followed by flash chromatography using a gradient of 30-70% EtOAc in heptane to give isopropyl 6-(4-{[(4-chlorobenzypsulfonyl]carbamoyl}piperidin-1-yl)-5-cyano-2-methoxynicotinate. Yield: 20 mg (13%).

1H-NMR (500 MHz, CDCl3): 1.34 (6H, d), 1.78-1.96 (4H, m), 2.50 (1H, m), 3.19 (2H, m), 4.01 (3H, s), 4.62-4.69 (4H, m), 5.16 (1H, m), 7.25-7.40 (4H, m), 8.31 (1H, s).

GTPγS(IC₅₀ μM): 0.011

### Example 56

### Isopropyl 5-cyano-6-(4-{[(4-fluorobenzyl)sulfonyl]carbamoyl}piperidin-1-yl)-2-methoxynicotinate

Prepared according to the procedure described in Example 55(c) using 1-(4-chlorophenyl)methanesulfonamide (57 mg, 0.302 mmol) and 1-[3-cyano-5-(isopropoxycarbonyl)-6-methoxypyridin-2-yl]piperidine-4-carboxylic acid (100 mg, 0.288 mmol. Yield: 5 mg (3%).

¹H-NMR (500 MHz, CDCl₃): δ 1.32 (6H, d), 1.75-1.95 (4H, m), 2.47 (1H, m), 3.18 (2H, m), 3.99 (3H, s), 4.61-4.68 (4H, m), 5.16 (1H, m), 7.08 (2H, dd), 7.33 (2H, dd), 8.30 (1H, s).

GTPγS(IC₅₀ μM): 0.025

### Example 57

### Ethyl 6-{3-[(benzylsulfonyl)carbamoyl]azetidin-1-yl}-5-cyano-2-(methylthio)nicotinate

### (a) Ethyl 6-[3-(tert-butoxycarbonyl)azetidin-1-yl]-5-cyano-2-{[(trifluoromethyl)sulfonyl]oxy}nicotinate

Tf₂(O) (100 mg, 0.35 mmol) was added to a cold(ice/water bath temperature) solution of ethyl 6-[3-(tert-butoxycarbonyl)azetidin-1-yl]-5-cyano-2-oxo-1,2-dihydropyridine-3-carboxylate (Example 2(e)) (100mg, 0.288 mmol) and TEA (150 mg, 1.48 mmol) in dry DCM (5 mL) and the mixture was stirred for 30 minutes. The solvent and excess regents were evaporated and NaHCO₃(aq) was added and the mixture was extracted with DCM(×3). The combined organic layer was run through a phase separator and evaporated to give ethyl 6-[3-(tert-butoxycarbonyl)azetidin-1-yl]-5-cyano-2-{[(trifluoromethyl)sulfonyl]oxy}nicotinate which was used in the next step without further purification.

### (b) Ethyl 6-[3-(tert-butoxycarbonyl)azetidin-1-yl]-5-cyano-2-(methylthio)nicotinate

A microwave vial was charged with DIPEA (74 mg, 0.576 mmol) , ethyl 6-[3-(tert-butoxycarbonyl)azetidin-1-yl]-5-cyano-2-{[(trifluoromethyl)sulfonyl]oxy}nicotinate (138 mg, 0.288 mmol), sodium methylthiolate (30 mg, 0.428 mmol) and THF (3 mL) and the mixture was heated to 140° C. for 5 minutes using microwave single node heating. NaHCO₃(aq) was added and the mixture was extracted with DCM(×3). The combined organic layer was run through a phase separator and evaporated to give ethyl 6-[3-(tert-butoxycarbonyl)azetidin-1-yl]-5-cyano-2-(methylthio)nicotinate which was used in the next step without further purification. Yield assumed quantitative.

MS m/z: 378 (M+1).

### (c) 1-[3-cyano-5-(ethoxycarbonyl)-6-(methylthio)pyridin-2-yl]azetidine-3-carboxylic acid

A solution of ethyl 6-[3-(tert-butoxycarbonyl)azetidin-1-yl]-5-cyano-2-(methylthio)nicotinate (109 mg, 0.288 mmol) in DCM/TFA (4/3, 7 mL) was stirred at r.t. for 1.5 hours. The solvent and excess TFA was removed in vaccuo to give 143-cyano-5-(ethoxycarbonyl)-6-(methylthio)pyridin-2-yl]azetidine-3-carboxylic acid which was used is in the next step without further purification.

MS m/z: 322 (M+1), 320 (M−1).

### (d) Ethyl 6-{3-[(benzylsulfonyl)carbamoyl]azetidin-1-yl}-5-cyano-2-(methylthio)nicotinate

DIPEA (185 mg, 1.43 mmol) was added to a solution of 1-phenylmethanesulfonamide (52 mg, 0.304 mmol), PyBrop (164 mg, 0.245 mmol) and 143-cyano-5-(ethoxycarbonyl)-6-(methylthio)pyridin-2-yl]azetidine-3-carboxylic acid (92 mg, 0.288 mmol) in THF (% mL) and the mixture was stirred at r.t. for 72 hours. Additional PyBrop and 1-phenylmethanesulfonamide were added until complete consumption of the starting acid by LC-MS.

NaHCO₃ (aq) was added and the mixture was extracted with DCM (×3).The combined organic phase was run through a phase separator and evaporated to give a crude material which was purified by HPLC (Kromasil C₈, 10 mm, using a gradient of increasing MeCN with a second eluent (0.1 M NH₄OAc/MeCN, 95/5)) to give Ethyl 6-{3-[(benzylsulfonyl)carbamoyl]azetidin-1-yl}-5-cyano-2-(methylthio)nicotinate. Yield: 35 mg (25%).

¹H NMR (500 MHz, DMSO-d₆): δ 1.30 (3H, t, J=7.1 Hz), 2.42 (3H, s), 3.54-3.61 (1H, m), 4.24 (2H, q, J=7.1 Hz), 4.31-4.40 (2H, m), 4.41-4.51 (2H, m), 4.75 (2H, s), 7.33-7.41 (5H, m), 8.25 (1H, s), 11.82 (1H, br s).

MS m/z: 475 (M+1), 473 (M−1).

GTPγS(IC₅₀ μM): 0.018

### Example 58

### Ethyl 6-{4-[(benzylsulfonyl)carbamoyl]piperidin-1-yl}-5-cyano-2-(methylthio)nicotinate

### (a) Ethyl 6-[4-(tert-butoxycarbonyl)piperidin-1-yl]-5-cyano-2-{[(trifluoromethyl)sulfonyl]oxy}nicotinate

Tf₂(O) (0.3 mL, 1.78 mmol) was added to a cold (ice/water bath temperature) mixture of ethyl 6-[4-(tert-butoxycarbonyl)piperidin-1-yl]-5-cyano-2-oxo-1,2-dihydropyridine-3-carboxylate (See Example 7(b)) (626 mg, 1.67 mmol) and TEA (0.5 mL, 3.59 mmol) in DCM (10 mL) and the mixture was stirred for 40 minutes.

The mixture was concentrated under reduced pressure and the crude was used in the next step without further purification.

MS m/z: 508 (M+1).

### (b) 1-[3-cyano-5-(ethoxycarbonyl)-6-{[(trifluoromethyl)sulfonyl]oxy}pyridin-2-yl]piperidine-4-carboxylic acid

TFA (10 mL) was added to a solution of crude ethyl 6-[4-(tert-butoxycarbonyl)piperidin-1-yl]-5-cyano-2-{[(trifluoromethyl)sulfonyl]oxy}nicotinate (3.99 g, 7.86 mmol) in DCM (20 mL) and the reaction mixture was stirred at r.t for 30 minutes. The mixture was concentrated under reduced pressure and the crude product was used in the next step without further purification. Yield assumed quantitative.

### (c) Ethyl 2-(1H-benzotriazol-1-yloxy)-6-{4-[(benzylsulfonyl)carbamoyl]piperidin-1-yl}-5-cyanonicotinate

DIPEA (5 mL, 28.7 mmol) was added to a solution of crude 1-[3-cyano-5-(ethoxycarbonyl)-6-{[(trifluoromethyl)sulfonyl]oxy}pyridin-2-yl]piperidine-4-carboxylic acid (3.55 g, 7.86 mmol) and TBTU (3.66 g, 11.4 mmol) in dry DCM (25 mL). The mixture was stirred at r.t for 100 min. 1-phenylmethanesulfonamide (1.35 g, 7.88 mmol) was added and the reaction mixture was stirred at r.t for an additional 20 h. NaHCO₃(aq) was added and the mixture was extracted with DCM (×3). The combined organics was run through a phase separator and concentrated under reduced pressure. The crude product was purified by preparative HPLC (Kromasil C₈ 10 μm, 50.8×300 mm, using a gradient of 20-60% CH₃CN/0.1 M NH₄OAc) to give ethyl 2-(1H-benzotriazol-1-yloxy)-6-{4-[(benzylsulfonyl)carbamoyl]piperidin-1-yl}-5-cyanonicotinate as a white solid after freeze drying from water. Yield: 1.79 g (39%).

MS m/z: 590 (M+1), 588 (M−1).

### (d) Ethyl 6-{4-[(benzylsulfonyl)carbamoyl]piperidin-1-yl}-5-cyano-2-(methylthio)nicotinate

A microwave vial was charged with DIPEA (592 mg, 4.58 mmol), ethyl 2-(1H-benzotriazol-1-yloxy)-6-{4-[(benzylsulfonyl)carbamoyl]piperidin-1-yl}-5-cyanonicotinate (900 mg, 1.526 mmol), sodium methylthiolate (214 mg, 3.053 mmol) and EtOH and the mixture was heated to 120° C. for 5 minutes using microwave single node heating. The solvent was evaporated and the crude product was purified by HPLC (Kromasil C₈, 10 μm, using a gradient of MeCN with an acidic second eluent (H₂O/MeCN/FA, 95/5/0.2)) to give ethyl 6-{4-[(benzylsulfonyl)carbamoyl]piperidin-1-yl}-5-cyano-2-(methylthio)nicotinate. Yield: 230 mg (29%).

¹H NMR (500 MHz, DMSO-d₆): δ 1.30 (3H, t, J=7.1 Hz), 1.62-1.72 (2H, m), 1.82-1.88 (2H, m), 2.44 (3H, s), 2.57-2.65 (1H, m), 3.17-3.25 (2H,m), 4.25 (2H, q, J=7.1 Hz), 4.54-4.59 (2H, m), 4.70 (2H, s), 7.28-7.31 (2H, m), 7.38-7.42 (3H, m), 8.28 (1H, s), 11.61 (1H, br s).

MS m/z: 503 (M+1), 501 (M−1).

GTPγS(IC₅₀ μM): 0.0077

### Example 59

### Ethyl 6-{4-[(benzylsulfonyl)carbamoyl]piperidin-1-yl}-2,5-dichloronicotinate

### (a) Ethyl 6-{4-[(benzylsulfonyl)carbamoyl]piperidin-1-yl}-2-chloronicotinate

A micro wave vial was charged with DIPEA (2.73 g, 21.1 mmol), ethyl 2,6-dichloronicotinate (Example 43(a)) (1.547 g, 7.03 mmol), N-(benzylsulfonyl)piperidine-4-carboxamide (Example 6(d)) (2.28 g, 8.08 mmol) and DMF and the mixture was heated to 120° C. for 10 minutes followed by 150° C. for 10 minutes using microwave single node heating.

Ratio of the two possible regioisomers was ca. 1:1 together with some bis-addition adduct. The crude product was purified by first using HPLC (Kromasil C₈, 10 μm, using a gradient of MeCN with an acidic second eluent (H₂O/MeCN/AcOH, 95/5/0.1)) followed by flash chromatography using a stepwise gradient of heptane/EtOAc 1/1 then heptane/EtOAc 1/1+0.15% FA and finally heptane/EtOAc 1/2+0.15% FA. (Rf product (heptane/EtOAc 1/2+0.15% FA)=0.47) to give ethyl 6-{4-[(benzylsulfonyl)carbamoyl]piperidin-1-yl}-2-chloronicotinate. Yield: 610 mg (19%).

### (b) Ethyl 6-{4-[(benzylsulfonyl)carbamoyl]piperidin-1-yl}-2,5-dichloronicotinate

A micro wave vial was charged with ethyl 6-{4-[(benzylsulfonyl)carbamoyl]piperidin-1-y}-2-chloronicotinate (70 mg, 0.15 mmol), NCS (40 mg, 0.30 mmol) and MeCN (1.2 mL) and the mixture was heated to 100° C. for 30 minutes using microwave single node heating. Evaporation of the solvent gave a crude product which was purified by flash chromatography using a stepwise gradient of heptane/EtOAc 3/1 then heptane/EtOAc 2/1 and finally heptane/EtOAc 2/1+0.1% FA to give Ethyl 6-{4-[(benzylsulfonyl)carbamoyl]piperidin-1-yl}-2,5-dichloronicotinate. Yield: 28 mg (37%).

¹H NMR (500 MHz, d₆-DMSO): δ 1.31 (3H, t, J=7.1 Hz), 1.63-1.76 (2H, m), 1.79-1.87 (2H, m), 2.48-2.55 (1H, m, hidden under DMSO signal), 2.92-3.01 (2H, m), 4.07-4.15 (2H, m), 4.30 (2H, q, J=7.1 Hz), 4.72 (2H, s), 7.29-7.34 (2H, m), 7.40-7.45 (3H, m), 8.16 (1H, s), 11.61 (1H, s).

MS m/z: 500 (M+1)

GTPγS(IC₅₀ μM): 0.033

### Example 60

### Isopropyl 6-{4-[(benzylsulfonyl)carbamoyl]piperidin-1-yl}-5-cyano-2-methoxynicotinate

Prepared according to the procedure described in Example 55(c) from 1-[3-Cyano-5-(isopropoxycarbonyl)-6-methoxypyridin-2-yl]piperidine-4-carboxylic acid (Example 55(b)) (100 mg, 0.288 mmol), and 1-phenylmethanesulfonamide (52 mg, 0.302 mmol). Yield: 25 mg (17%).

¹H-NMR (500 MHz, CDCl₃): δ 1.32 (6H, d), 1.75-1.90 (4H, m), 2.46 (1H, septett), 3.15 (2H, m), 3.98 (3H, s), 4.58-4.66 (4H, m), 5.14 (1H, m), 7.29-7.40 (5H, m), 8.28 (1H, s).

GTPγS(IC₅₀ μM): 0.027

### Example 61

### N-(Benzylsulfonyl)-1-[3-cyano-6-(methylthio)-5-pentanoylpyridin-2-yl]piperidine-4-carboxamide

### (a) tert-Butyl 6-[4-(tert-butoxycarbonyl)piperidin-1-yl]-5-cyano-2-(methylthio)nicotinate

A microwave vial was charged with ethyl 6-[4-(tert-butoxycarbonyl)piperidin-1-yl]-5-cyano-2-{[(trifluoromethyl)sulfonyl]oxy}nicotinate (Example 58(a)) (139 mg, 0.274 mmol), sodium methanethiolate (24.4 mg, 0.348 mmol), Pd₂(dba)₃ (22.6 mg, 0.025 mmol), Xantphos (15.4 mg, 0.027 mmol), dry dioxane (3 mL) and DIPEA(0.1 ml, 0.574 mmol). The reaction mixture was heated to 120° C. for 5min using microwave single node irradiation. LCMS showed full conversion. NaHCO₃(aq) was added and the mixture was extracted with DCM(×3). The combined organic layer was run through a phase separator and evaporated. The crude product was purified by preparative HPLC (Kromasil C₈ 10 μm, 21.5×250 mm, using a gradient of MeCN with a second eluent 0.1M NH4OAc/MeCN 95/5)) to give tert-butyl 6-[4-(tert-butoxycarbonyl)piperidin-1-yl]-5-cyano-2-(methylthio)nicotinate.

Yield: 69 mg (62%).

### (b) 6-[4-(tert-butoxycarbonyl)piperidin-1-yl]-5-cyano-2-(methylthio)nicotinic acid

A microwave vial was charged with 1M NaOH (6 mL, 6 mmol) , tert-butyl 6-[4-(tert-butoxycarbonyl)piperidin-1-yl]-5-cyano-2-(methylthio)nicotinate (1.36 g, 3.37 mmol), THF (6 mL) and EtOH (6 mL). The reaction mixture was heated to 60° C. for 5 minutes in a single node microwave oven. The reaction mixture was concentrated under reduced pressure and acetic acid (0.36 mL, 6.29 mmol) and water was added. The solid was filtered off and washed with 2-propanol/DEE (1:1) and dried under reduced pressure to give the product as a off white solid (203 mg). The filtrate was evaporated, NaHCO₃(aq) was added and the mixture was extracted with DCM (×3). The combined organics was run through a phase separator and evaporated. The crude product was purified by preparative HPLC (Kromasil C₈ 10 μm, 21.5×250 mm, using an increasing gradient of MeCN with a second acidic eluent H₂O/MeCN/FA 95/5/0.2)) to give an additional 366 mg of 6-[4-(tert-butoxycarbonyl)piperidin-1-yl]-5-cyano-2-(methylthio)nicotinic acid as a white solid. Yield: 569 mg (45%).

¹H NMR (400 MHz, DMSO-d₆): δ 1.39 (9H, s), 1.54-1.66 (2H, m), 1.87-1.95 (2H, m), 2.37 (3H, s), 2.54-2.64 (1H, m), 3.24-3.36 (2H, m, consealed by DMSO signal at 3.3), 4.38-4.47 (2H, m), 8.20 (1H, s), 12.97 (1H, br s).

MS m/z: 378.0 (M+1), 376.2 (M−1).

### (c) tert-Butyl 1-[3-cyano-5-(fluorocarbonyl)-6-(methylthio)pyridin-2-yl]piperidine-4-carboxylate

Dry pyridine (0.15 mL, 1.86 mmol) and cyanuric fluoride (0.15 mL, 1.78 mmol) were added to a suspension of 6-[4-(tert-butoxycarbonyl)piperidin-1-yl]-5-cyano-2-methylthio)nicotinic acid (569 mg, 1.51 mmol) in DCM (20 mL). The reaction mixture was stirred at r.t for 30 minutes. LCMS showed 10% acid (sample quenched with 1% DIPEA in dry Me0H). The reaction mixture was stirred at r.t for another 50 minutes. LCMS still showed 10% acid but 20% anhydrid had been formed. Dry pyridine (0.02 mL, 0.25 mmol) and cyanuric fluoride (0.02 mL, 0.24 mmol) were added. The reaction mixture was stirred at r.t for an additional 15 minutes. LCMS showed 4% acid left. The solid was filtered off and washed with dry DCM. Water was added to the filtrate, the organic layer was separated and the aqueous layer was extracted with DCM. The combined organics was run through a phase separator and evaporated to give the crude tert-butyl 1-[3-cyano-5-(fluorocarbonyl)-6-(methylthio)pyridin-2-yl]piperidine-4-carboxylate as a solid. The crude was used in the next step without further purification, yield assumed quantitative.

¹H NMR (400 MHz, CDCl₃): δ 1.46 (9H, s), 1.78-1.90 (2H, m), 2.01-2.09 (2H, m), 2.49 (3H, s), 2.54 (1H, m), 3.38-3.48 (2H, m), 4.57-4.66 (2H, m), 8.18 (1H, s). (¹H NMR showed product/ anhydride in a ratio 4:1.)

MS m/z: 392 (M+1). (identified as methylester after quench with MeOH/DIPEA)

### (d) di-tert-Butyl({6-[4-(tert-butoxycarbonyl)piperidin-1-yl]-5-cyano-2-(methylthio)pyridin-3-yl}carbonyl)(propyl)malonate

A suspension of the crude tert-butyl 1-[3-cyano-5-(fluorocarbonyl)-6-(methylthio)pyridin-2-yl]piperidine-4-carboxylate from above (1.51 mmol) in dry THF (12 mL) was added to a solution of di-tert-butyl propylmalonate (541 mg, 2.09 mmol) in dry THF (8 mL) and sodium pentoxide (326 mg, 2.96 mmol) was added to the mixture which was cooled with an cold water bath. The reaction mixture was stirred at r.t for 1.5 hours. TFA (0.8 mL, 10.4 mmol) was added and the mixture was evaporated. Water was added and the mixture was extracted with DCM (×3). The combined organics was run through a phase separator and evaporated. The crude product was purified by preparative HPLC (Kromasil C₈ 10 μm, 21.5×250 mm, using an increasing gradient of MeCN with a second acidic eluent H₂O/MeCN/FA 95/5/0.2)) to give di-tert-butyl ({6-[4-(tert-butoxycarbonyl)piperidin-1-yl]-5-cyano-2-(methylthio)pyridin-3-yl}carbonyl)(propyl)malonate as a white solid. Yield: 366 mg (39% over 2 steps).

¹H NMR (400 MHz, CDCl₃): δ 0.95 (3H, t, J=7.4 Hz), 1.45 (18H, s), 1.46 (9H, s), 1.75-1.87 (2H, m), 1.98-2.06 (2H, m), 2.11-2.18 (2H, m), 2.42 (3H, s), 2.51-2.59 (1H, m), 3.30-3.39 (2H, m), 4.54-4.61 (2H, m), 8.18 (1H, s).

MS m/z: 618 (M+1).

### (e) 1-[5-(2-carboxypentanoyl)-3-cyano-6-(methylthio)pyridin-2-yl]piperidine-4-carboxylic acid

TFA (4 mL, 51.9 mmol) was added to a solution of di-tert-butyl ({6-[4-(tert-butoxycarbonyl)piperidin-1-yl]-5-cyano-2-(methylthio)pyridin-3-yl}carbonyl)(propyl)malonate (360 mg, 0.58 mmol) in DCM (5 mL).The reaction mixture was stirred at r.t for 1.5 hours and evaporated to give 1-[5-(2-carboxypentanoyl)-3-cyano-6-(methylthio)pyridin-2-yl]piperidine-4-carboxylic acid as a solid. The crude was used in the next step without further purification. Yield assumed quantitative.

¹H NMR (400 MHz, CDCl₃): δ 0.94 (3H, t, J=7.3 Hz), 1.32-1.41 (2H, m), 1.83-1.93 (2H, m), 1.93-2.01 (2H, m), 2.09-2.17 (2H, m), 2.44 (3H, s), 2.73-2.82 (1H, m), 3.35-3.48 (2H, m), 4.19 (1H, t, J=7.1 Hz), 4.62-4.70 (2H, m), 8.20 (1H, s).

### (f) 1-[3-cyano-6-(methylthio)-5-pentanoylpyridin-2-yl]piperidine-4-carboxylic acid

A microwave vial was charged with 1-[5-(2-carboxypentanoyl)-3-cyano-6-(methylthio)pyridin-2-yl]piperidine-4-carboxylic acid (0.583 mmol) and CH₃CN (2.5 mL). The reaction mixture was heated to 120° C. for 10minutes in a single node microwave oven. LCMS showed complete conversion to the product. The reaction mixture was evaporated and co-evaporated from DCM to give crude 1-[3-cyano-6-(methylthio)-5-pentanoylpyridin-2-yl]piperidine-4-carboxylic acid as a solid. Yield assumed quantitative.

¹H NMR (400 MHz, DMSO-d₆): δ 0.87 (3H, t, J=7.4 Hz), 1.23-1.33 (2H, m), 1.45-1.54 (2H, m), 1.56-1.68 (2H, m), 1.90-1.99 (2H, m), 2.34 (3H, s), 2.57-2.66 (1H, m), 2.87 (2H, t, J=7.3 Hz), 3.27-3.35 (2H, m, consealed by DMSO signal at 3.31), 4.43-4.50 (2H, m), 8.54 (1H, s), 12.31 (1H, br s).

MS m/z: 362 (M+1), 360 (M−1).

### (g) N-(Benzylsulfonyl)-1-[3-cyano-6-(methylthio)-5-pentanoylpyridin-2-yl]piperidine-4-carboxamide

DIPEA (0.2 mL, 1.15 mmol) was added to a suspension of the crude 1-[3-cyano-6-(methylthio)-5-pentanoylpyridin-2-yl]piperidine-4-carboxylic acid (0.29 mmol), and TBTU (144 mg, 0.45 mmol) in dry DCM (4 mL) and and the reaction mixture was stirred at r.t for 2h before 1-phenylmethanesulfonamide (67 mg, 0.39 mmol) was added and the reaction mixture was stirred at r.t over night. NaHCO₃(aq) was added and the mixture was extracted with DCM (×3). The combined organics was run through a phase separator and evaporated. The crude product was purified by preparative HPLC (Kromasil C₈ 10μm, 50×300 mm, using an increasing gradient of MeCN with a second acidic eluent H₂O/MeCN/FA 95/5/0.2)) to give N-(Benzylsulfonyl)-1-[3-cyano-6-(methylthio)-5-pentanoylpyridin-2-yl]piperidine-4-carboxamide as a white solid. Yield: 114 mg (76% over 3 steps).

¹H NMR (400 MHz, DMSO-d₆): δ 0.88 (3H, t, J=7.4 Hz), 1.25-1.33 (2H, m), 1.47-1.54 (2H, m), 1.60-1.70 (2H, m), 1.80-1.86 (2H, m), 2.37 (3H, s), 2.56-2.63 (1H, m), 2.88 (2H, t, J=7.3 Hz), 3.15-3.23 (2H, m), 4.52-4.60 (2H, m), 4.67 (2H, s), 7.26-7.30 (2H, m), 7.36-7.40 (3H, m), 8.56 (1H, s), 11.59 (1H, br s).

MS m/z: 515 (M+1), 513 (M−1).

### Example 61

### 1-[3-cyano-6-(methylthio)-5-pentanoylpyridin-2-yl]-N-[(4-methoxybenzyl)sulfonyl]piperidine-4-carboxamide

Prepared according to the procedure described in Example 60(g) using 1-[3-cyano-6-(methylthio)-5-pentanoylpyridin-2-yl]piperidine-4-carboxylic acid (0.29 mmol) and 1-[4-(methoxy)phenyl]sulfonamide (83 mg, 0.41 mmol). Yield: 129 mg (81% over 3 steps).

¹H NMR (400 MHz, DMSO-d₆): 0.87 (3H, t, J=7.4 Hz), 1.23-1.34 (2H, m), 1.45-1.55 (2H, m), 1.58-1.71 (2H, m), 1.79-1.87 (2H, m), 2.36 (3H, s), 2.55-2.64 (1H, m), 2.87 (2H, t, J=7.3 Hz), 3.13-3.24 (2H, m), 3.74 (3H, s), 4.52-4.61 (2H, m), 4.59 (2H, s), 6.93 (2H, d part of an AB system, J_{AB}=8.6 Hz), 7.18 (2H, d part of an AB system, J_{AB}=8.6 Hz), 8.55 (1H, s), 11.53 (1H, br s).

MS m/z: 545 (M+1), 543 (M−1).

1. A compound of formula I or a pharmaceutically acceptable salt thereof: 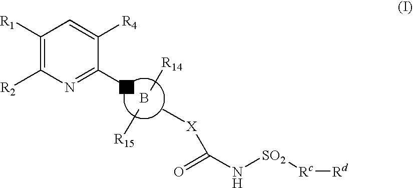
wherein:
R₁ represents R₆OC(O), R₇C(O), R₁₆SC(O), R₁₇S, R₁₈C(S) or a group gII 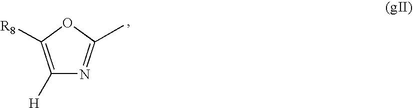
R₂ represents CN, halogen, (C₄-C₈)alkyl optionally interrupted by oxygen and/or optionally substituted by OH, aryl, cycloalkyl, or heterocyclyl; or R₂ represents (C₂-C₃)alkyl interrupted by oxygen; or R₂ represents (C₁-C₃)alkyl substituted by one or more of OH, aryl, aryl(C₁-C₃)alkyloxy, cycloalkyl and heterocyclyl, with the proviso that any such OH group must be at least 2 carbon atoms away from any oxygen; or R₂ represents unsubstituted (C₁-C₁₂)alkoxy, (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, unsubstituted (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl;
R₄ represents H, CN, a halogen atom, (C₁-C₁₂)alkyl optionally interrupted by oxygen and/or optionally substituted by OH, COOH, (C₁-C₆)alkoxycarbonyl, or one or more halogen atoms; or R₄ represents hydroxy(C₁-C₁₂)alkyl, (C₁-C₁₂)alkoxy wherein the alkoxy group may optionally be substituted by one or more halogen atoms, OH and/or COOH and/or (C₁-C₆)alkoxycarbonyl; or R₄ represents aryl(C₁-C₆)alkyl, (C₁-C₁₂)alkylsulfinyl, (C₁-C₁₂)alkylsulfonyl, (C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₃-C₆)cycloalkyl(C₁-C₁₂)alkoxy, aryl(C₁-C₆)alkoxy or a group of formula NR^{a(4)}R^{b(4)} in which R^{a(4)} and R^{b(4)} independently represent H, (C₁-C₁₂)alkyl, or (C₁-C₁₂)alkylC(O) or R^{a(4)} and R^{b(4)} together with the nitrogen atom represent piperidine, pyrrolidine, azetidine or aziridine;
R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or R₆ represents (C₃-C₆)cycloalkyl, hydroxy(C₂-C₁₂)alkyl, aryl or heterocyclyl;
R₇ represents (C₁-C₁₂)alkyl optionally interrupted by oxygen, and/or optionally substituted by OH, aryl, cycloalkyl, heterocyclyl or one or more halogen atoms; or R₇ represents (C₃-C₆)cycloalkyl, hydroxy(C₁-C₁₂)alkyl, aryl or heterocyclyl;
R₈ represents H, (C₁-C₁₂)alkyl optionally interrupted by oxygen, and/or optionally substituted by aryl, cycloalkyl, heterocyclyl or one or more halogen atoms; or R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl;
R₁₄ represents H, OH with the proviso that the OH group must be at least 2 carbon atoms away from any heteroatom in the B ring/ring system, (C₁-C₈)alkyl optionally interrupted by oxygen and/or optionally substituted by one or more of OH, COOH and COOR^{e}; wherein R^{e} represents aryl, cycloalkyl, heterocyclyl or (C₁-C₈)alkyl optionally substituted by one or more of halogen atom(s), OH, aryl, cycloalkyl and heterocyclyl; or R₁₄ represents aryl, aryl(C₁-C₈)alkyl, aryl(C₁-C₃)alkoxy, heterocyclyl, a halogen atom, (C₃-C₆)cycloalkyl, (C₃-C₆)cycloalkyl(C₁-C₈)alkoxy, hydroxy(C₁-C₈)alkyl, (C₁-C₈)alkoxy, (C₃-C₆)cycloalkoxy, (C₁-C₈)alkylsulfinyl, (C₁-C₈)alkylsulfonyl, (C₁-C₈)alkylthio, (C₃-C₆)cycloalkylthio, or a group of formula NR^{a(14)}R^{b(14)} in which R^{a(14)} and R^{b(14)} independently represent H, (C₁-C₈)alkyl, (C₁-C₈)alkylC(O), (C₁-C₈)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen atom(s), OH, aryl, cycloalkyl and heterocyclyl; or R₁₅ represents aryl, aryl(C₁-C₈)alkyl, aryl(C₁-C₃)alkoxy, heterocyclyl, a halogen atom, (C₃-C₆)cycloalkyl, (C₃-C₆)cycloalkyl(C₁-C₈)alkoxy, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, or a group of formula NR^{a(15)}R^{b(15)} in which R^{a(15)} and R^{b(15)} independently represent H, (C₁-C₁₂)alkyl, (C₁-C₁₂)alkylC(O), (C₁-C₁₂)alkoxyC(O) or R^{a(15)} and R^{b(15)} together with the nitrogen atom represent piperidine, pyrrolidine, azetidine or aziridine;
R₁₆ represents (C₁-C₁₂)alkyl optionally interrupted by oxygen and/or optionally substituted by OH, aryl, cycloalkyl, heterocyclyl or one or more halogen atoms; or R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;
R₁₇ represents (C₁-C₁₂)alkyl optionally interrupted by oxygen and/or optionally substituted by OH, aryl, cycloalkyl, heterocyclyl or one or more halogen atoms; or R₁₇ represents (C₃-C₆)cycloalkyl, hydroxy(C₁-C₁₂)alkyl,(C₁-C₁₂)alkoxy, (C₃-C₆)cycloalkoxy, aryl or heterocyclyl;
R₁₈ represents (C₁-C₁₂)alkyl optionally interrupted by oxygen and/or optionally substituted by OH, aryl, cycloalkyl, heterocyclyl or one or more halogen atoms; or R₁₈ represents (C₃-C₆)cycloalkyl, hydroxy(C₁-C₁₂)alkyl,(C₁-C₁₂)alkoxy, (C₃-C₆)cycloalkoxy, aryl or heterocyclyl;
R^{c} is a direct bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; or R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above;
R₁₉ represents H or (C₁-C₄)alkyl;
R^{d} represents (C₁-C₁₂)alkyl, (C₃-C₈)cycloalkyl, aryl or heterocyclyl, and anyone of these groups optionally substituted with one or more halogen atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;
X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; or X may represent a group (—CH₂-)n wherein n=2-6, which optionally is unsaturated and/or substituted by one or more substituent chosen among halogen, hydroxyl or (C₁-C₆)alkyl; and
B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions; wherein substituents R₁₄ and R₁₅ are connected to the B ring/ring system in such a way that no quarternary ammonium compounds are formed (by these connections).

2. A compound according to claim 1 wherein:
R₂ represents CN, halogen, (C₄-C₆)alkyl optionally interrupted by oxygen and/or optionally substituted by OH, aryl, cycloalkyl, heterocyclyl; or R₂ represents (C₂-C₃)alkyl interrupted by oxygen; or R₂ represents (C₁-C₃)alkyl substituted by one or more of OH, aryl, aryl(C₁-C₃)alkyloxy, cycloalkyl and heterocyclyl, with the proviso that any such OH group must be at least 2 carbon atoms away from any oxygen; or R₂ represents unsubstituted (C₁-C₆)alkoxy, (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, unsubstituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, or (C₃-C₆)cycloalkyl(C₁-C₆)alkylsulfonyl;
R₄ represents H, CN, a halogen atom, (C₁-C₆)alkyl optionally interrupted by oxygen and/or optionally substituted by OH, COOH, (C₁-C₆)alkoxycarbonyl, or one or more halogen atoms; or R₄ represents hydroxy(C₁-C₆)alkyl, (C₁-C₆)alkoxy wherein the alkoxy group may optionally be substituted by one or more halogen atoms, OH and/or COOH and/or (C₁-C₆)alkoxycarbonyl; or R₄ represents aryl(C₁-C₆)alkyl, (C₁-C₆)alkylsulfinyl, (C₁-C₆)alkylsulfonyl, (C₁-C₆)alkylthio, (C₃-C₆)cycloalkyl(C₁-C₆)alkylsulfinyl, (C₃-C₆)cycloalkyl(C₁-C₆)alkylsulfonyl, (C₃-C₆)cycloalkyl(C₁-C₆)alkoxy, aryl(C₁-C₆)alkoxy or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or R₆ represents (C₃-C₆)cycloalkyl, hydroxy(C₂-C₆)alkyl, aryl or heterocyclyl;
R₇ represents (C₁-C₆)alkyl optionally interrupted by oxygen, and/or optionally substituted by OH, aryl, cycloalkyl, heterocyclyl or one or more halogen atoms; or R₇ represents (C₃-C₆)cycloalkyl, hydroxy(C₁-C₆)alkyl, aryl or heterocyclyl;
R₈ represents H, (C₁-C₆)alkyl optionally interrupted by oxygen, and/or optionally substituted by aryl, cycloalkyl, heterocyclyl or one or more halogen atoms; or R₈ represents (C₃-C₆)cycloalkyl, hydroxy(C₁-C₆)alkyl, (C₁-C₆)alkoxy, (C₃-C₆)cycloalkoxy, aryl, heterocyclyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atom(s), OH, aryl, cycloalkyl and heterocyclyl; or R₁₄ represents aryl, aryl(C₁-C₆)alkyl, aryl(C₁-C₃)alkoxy, heterocyclyl, a halogen atom, (C₃-C₆)cycloalkyl, (C₃-C₆)cycloalkyl(C₁-C₆)alkoxy, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atom(s), OH, aryl, cycloalkyl and heterocyclyl; or R₁₅ represents aryl, aryl(C₁-C₆)alkyl, aryl(C₁-C₃)alkoxy, heterocyclyl, a halogen atom, (C₃-C₆)cycloalkyl, (C₃-C₆)cycloalkyl(C₁-C₆)alkoxy, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;
R₁₆ represents (C₁-C₆)alkyl optionally interrupted by oxygen and/or optionally substituted by OH, aryl, cycloalkyl, heterocyclyl or one or more halogen atoms; or R₁₆ represents (C₃-C₆)cycloalkyl, hydroxy(C₂-C₆)alkyl, (C₁-C₆)alkoxy, (C₃-C₆)cycloalkoxy, aryl or heterocyclyl;
R₁₇ represents (C₁-C₆)alkyl optionally interrupted by oxygen and/or optionally substituted by OH, aryl, cycloalkyl, heterocyclyl or one or more halogen atoms; or R₁₇ represents (C₃-C₆)cycloalkyl, hydroxy(C₁-C₆)alkyl, (C₁-C₆)alkoxy, (C₃-C₆)cycloalkoxy, aryl or heterocyclyl;
R₁₈ represents (C₁-C₆)alkyl optionally interrupted by oxygen and/or optionally substituted by OH, aryl, cycloalkyl, heterocyclyl or one or more halogen atoms; or R₁₈ represents (C₃-C₆)cycloalkyl, hydroxy(C₁-C₆)alkyl, (C₁-C₆)alkoxy, (C₃-C₆)cycloalkoxy, aryl or heterocyclyl; and
R^{d} represents (C₁-C₆)alkyl, (C₃-C₆)cycloalkyl, aryl or heterocyclyl, and anyone of these groups optionally substituted with one or more halogen atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine.

3. A compound according to claim 2 wherein:
R₁ represents R₆OC(O), R₇C(O) or a group gII 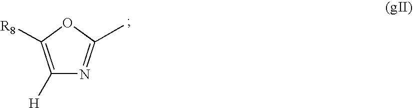
R₄ represents H, CN, a halogen atom, (C₁-C₆)alkyl optionally interrupted by oxygen and/or optionally substituted by OH, COOH, (C₁-C₆)alkoxycarbonyl, or one or more halogen atoms; or R₄ represents hydroxy(C₁-C₆)alkyl, (C₁-C₆)alkoxy wherein the alkoxy group may optionally be substituted by one or more halogen atoms, OH and/or COOH and/or (C₁-C₆)alkoxycarbonyl; or R₄ represents aryl(C₁-C₆)alkyl, (C₁-C₆)alkylthio, or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atom(s), OH, aryl, cycloalkyl and heterocyclyl; or R₁₄ represents aryl, heterocyclyl, a halogen atom, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine; and
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atom(s), OH, aryl, cycloalkyl and heterocyclyl; or R₁₄ represents aryl, heterocyclyl, a halogen atom, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine.

4. A compound according to claim 3 wherein:
R₂ represents CN, halogen, (C₄-C₆)alkyl optionally interrupted by oxygen and/or optionally substituted by OH, aryl, cycloalkyl, heterocyclyl; or R₂ represents (C₂-C₃)alkyl interrupted by oxygen; or R₂ represents (C₁-C₃)alkyl substituted by one or more of OH, aryl, aryl(C₁-C₃)alkyloxy, cycloalkyl and heterocyclyl, with the proviso that any such OH group must be at least 2 carbon atoms away from any oxygen; or R₂ represents unsubstituted (C₁-C₆)alkoxy, hydroxy(C₁-C₆)alkyl, (C₃-C₆)cycloalkoxy, unsubstituted (C₁-C₆)alkylthio, (C₃-C₆)cycloalkylthio, arylthio, aryl(C₁-C₆)alkylthio, heterocyclyl(C₁-C₆)alkylthio, or (C₃-C₆)cycloalkyl(C₁-C₆)alkylthio;
R₄ represents CN, a halogen atom; or R₄ represents hydroxy(C₁-C₆)alkyl, (C₁-C₆)alkoxy wherein the alkoxy group may optionally be substituted by one or more halogen atom(s), OH and/or COOH and/or (C₁-C₆)alkoxycarbonyl;
R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or R₆ represents (C₃-C₆)cycloalkyl or hydroxy(C₂-C₆)alkyl;
R₇ represents (C₁-C₆)alkyl optionally interrupted by oxygen, and/or optionally substituted by OH, aryl, cycloalkyl, heterocyclyl or one or more halogen atoms;
R₈ represents H, (C₁-C₆)alkyl optionally interrupted by oxygen, and/or optionally substituted by aryl, cycloalkyl, heterocyclyl or one or more halogen atoms;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atom(s), OH, aryl, cycloalkyl and heterocyclyl; or R₁₄ represents a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H;
R^{c} is a direct bond or represents an unsubstituted or monosubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl; or R^{c} represents imino (—NH—) or N-substituted imino (—NR₁₉—);
R₁₉ represents H or methyl;
R^{d} represents (C₁-C₆)alkyl, (C₃-C₆)cycloalkyl, aryl or heterocyclyl, and anyone of these groups optionally substituted with one or more halogen atoms and/or one or more of the following groups, CN, NO₂, (C₁-C₆)alkyl, (C₁-C₆)alkoxy, halogen substituted (C₁-C₆)alkyl; and X represents a single bond, imino (—NH—) or methylene (—CH₂—).

5. A compound according to claim 1 wherein:
R₁ is chosen from the group consisting of ethoxycarbonyl, ispropyloxycarbonyl, n-propylcarbonyl and n-butylcarbonyl;
R₂ is chosen from the group consisting of methoxy, ethoxy, methylthio, ethylthio, cyano, chloro, hydroxymethyl, ethoxymethyl, 2-methoxyethyl, (benzoyloxy)methyl, ((3,4-dimethoxybenzyl)oxy)methyl, 1H-1,2,4-triazol-1-yl-methyl, 1H-1,2,3-triazol-1-yl-methyl, and 1H-imidazol-1-yl-methyl;
R₃ is H;
R₄ is chosen from the group consisting of CN, chloro and fluoro;
R₆ is ethyl or isopropyl;
R₇ is n-propyl or n-butyl;
R₁₄ is H;
R₁₅ is H;
R^{c} is a single bond or methylene (—CH₂—);
R^{d} is chosen from the group consisting of phenyl, 2-fluorophenyl, 3-fluorophenyl, 4-fluorophenyl, 2-chlorophenyl, 4-chlorophenyl, 4-(trifluoromethyl)phenyl, 3,4-difluorophenyl, 2,4-difluorophenyl, 2,3-difluorophenyl, 2,4-dichlorophenyl, 2-chloro-4-fluorophenyl, 4-methoxy-phenyl and 4-chloro-2-fluorophenyl;
X is a single bond; and
B is 3 azetidin-1-ylene or 4-piperidin-1-ylene, and the substituents R₁₄ and R₁₅ are connected to the B ring/ring system, in such a way that no quarternary ammonium compounds are formed (by these connections).

6. A compound according to claim 1 which is of the formula (Ia):

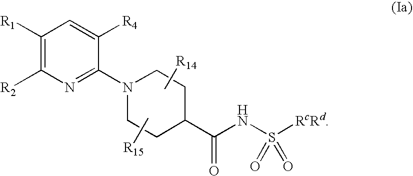

7. A compound according to claim 1 which is of the formula (Ib):

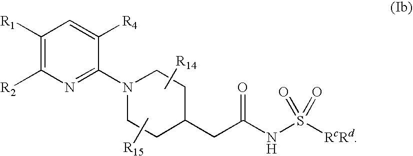

8. A compound according to claim 1 which is of the formula (Ic):

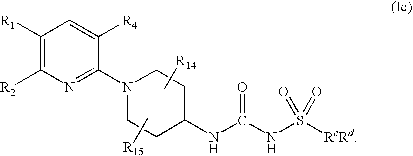

9. A compound according to claim 1 which is of the formula (Id):

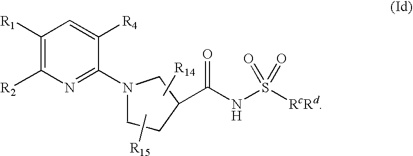

10. A compound according to claim 1 which is of the formula (Ie):

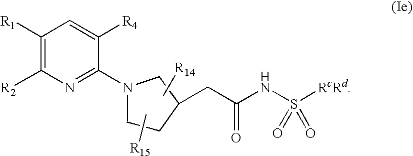

11. A compound according to claim 1 which is of the formula (If):

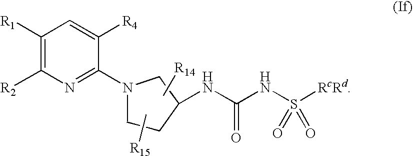

12. A compound according to claim 1 which is of the formula (Ig):

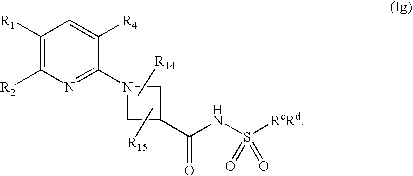

13. A compound according to claim 1 which is of the formula (Ih):

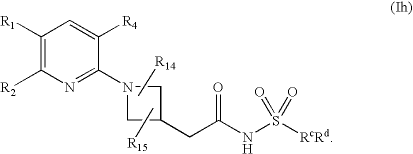

14. A compound according to claim 1 which is of the formula (Ii):

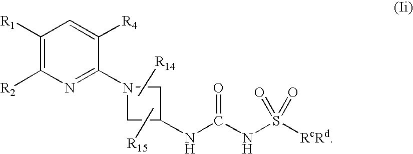

15. A compound according to claim 1 wherein R₁ represents R₆OC(O).

16. A compound according to claim 1 wherein R₁ represents R₇C(O) or a group gII

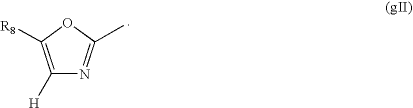

17. A compound according to claim 15 which is of the formula (Iaa):

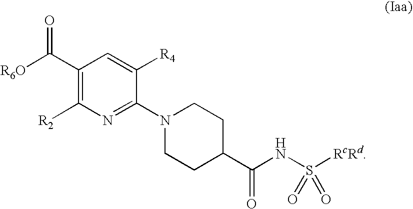

18. A compound according to claim 16 which is of the formula (Iab):

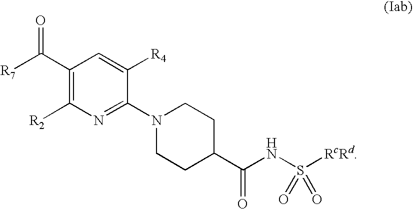

19. A compound according to claim 15 which is of the formula (Igg):

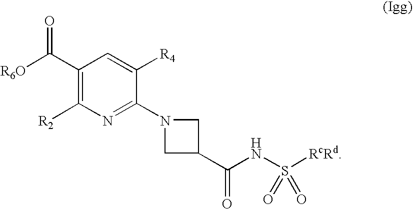

20. A compound selected from:
ethyl 6-{4-[(benzylsulfonyl)carbamoyl]piperidin-1-yl}-5-cyano-2-methoxynicotinate
ethyl 6-{3-[(benzylsulfonyl)carbamoyl]azetidin-1-yl}-5-cyano-2-methoxynicotinate
ethyl 6-{4-[(benzylsulfonyl)carbamoyl]piperidin-1-yl}-5-cyano-2-ethoxynicotinate
ethyl 6-{4-[(benzylsulfonyl)carbamoyl]piperidin-1-yl}-5-cyano-2-(ethylthio)nicotinate
ethyl 6-{4-[(benzylsulfonyl)carbamoyl]piperidin-1-yl}-2,5-dicyanonicotinate
ethyl 6-{4-[(benzylsulfonyl)carbamoyl]piperidin-1-yl}-5-cyano-2-(hydroxymethyl)nicotinate
ethyl 5-cyano-2-methoxy-6-{4-[(phenylsulfonyl)carbamoyl]piperidin-1-yl}nicotinate
ethyl 5-cyano-6-(4-{[(2-fluorobenzyl)sulfonyl]carbamoyl}piperidin-1-yl)-2-methoxynicotinate
ethyl 6-(4-{[(2-chlorobenzyl)sulfonyl]carbamoyl}piperidin-1-yl)-5-cyano-2-methoxynicotinate
ethyl 5-cyano-6-(4-{[(3-fluorobenzyl)sulfonyl]carbamoyl}piperidin-1-yl)-2-methoxynicotinate
ethyl 5-cyano-6-(4-{[(4-fluorobenzyl)sulfonyl]carbamoyl}piperidin-1-yl)-2-methoxynicotinate
ethyl 6-(4-{[(4-chlorobenzyl)sulfonyl]carbamoyl}piperidin-1-yl)-5-cyano-2-methoxynicotinate
ethyl 5-cyano-2-methoxy-6-[4-({[4-(trifluoromethyl)benzyl]sulfonyl}carbamoyl)piperidin-1-yl]nicotinate
ethyl 5-cyano-6-(4-{[(3,4-difluorobenzyl)sulfonyl]carbamoyl}piperidin-1-yl)-2-methoxynicotinate
ethyl 5-cyano-6-(4-{[(2,4-dichlorobenzyl)sulfonyl]carbamoyl}piperidin-1-yl)-2-methoxynicotinate
ethyl 5-cyano-6-(4-{[(2,4-difluorobenzyl)sulfonyl]carbamoyl}piperidin-1-yl)-2-methoxynicotinate
ethyl 6-(4-{[(2-chloro-4-fluorobenzyl)sulfonyl]carbamoyl}piperidin-1-yl)-5-cyano-2-methoxynicotinate
ethyl 6-(4-{[(4-chloro-2-fluorobenzyl)sulfonyl]carbamoyl}piperidin-1-yl)-5-cyano-2-methoxynicotinate
ethyl 5-cyano-6-(4-{[(2,3-difluorobenzyl)sulfonyl]carbamoyl}piperidin-1-yl)-2-methoxynicotinate
ethyl 5-cyano-2-methoxy-6-{3-[(phenylsulfonyl)carbamoyl]azetidin-1-yl}nicotinate
ethyl 5-cyano-6-(3-{[(2-fluorobenzyl)sulfonyl]carbamoyl}azetidin-1-yl)-2-methoxynicotinate
ethyl 6-(3-{[(2-chlorobenzyl)sulfonyl]carbamoyl}azetidin-1-yl)-5-cyano-2-methoxynicotinate
ethyl 5-cyano-6-(3-{[(3-fluorobenzyl)sulfonyl]carbamoyl}azetidin-1-yl)-2-methoxynicotinate
ethyl 5-cyano-6-(3-{[(4-fluorobenzyl)sulfonyl]carbamoyl}azetidin-1-yl)-2-methoxynicotinate
ethyl 6-(3-{[(4-chlorobenzyl)sulfonyl]carbamoyl}azetidin-1-yl)-5-cyano-2-methoxynicotinate
ethyl 5-cyano-2-methoxy-6-[3-({[4-(trifluoromethyl)benzyl]sulfonyl}carbamoyl)azetidin-1-yl]nicotinate
ethyl 5-cyano-6-(3-{[(3,4-difluorobenzyl)sulfonyl]carbamoyl}azetidin-1-yl)-2-methoxynicotinate
ethyl 5-cyano-6-(3-{[(2,4-dichlorobenzyl)sulfonyl]carbamoyl}azetidin-1-yl)-2-methoxynicotinate
ethyl 5-cyano-6-(3-{[(2,4-difluorobenzyl)sulfonyl]carbamoyl}azetidin-1-yl)-2-methoxynicotinate
ethyl 6-(3-{[(2-chloro-4-fluorobenzyl)sulfonyl]carbamoyl}azetidin-1-yl)-5-cyano-2-methoxynicotinate
ethyl 6-(3-{[(4-chloro-2-fluorobenzyl)sulfonyl]carbamoyl}azetidin-1-yl)-5-cyano-2-methoxynicotinate
ethyl 5-cyano-6-(3-{[(2,3-difluorobenzyl)sulfonyl]carbamoyl}azetidin-1-yl)-2-methoxynicotinate
ethyl 6-{3-[(benzylsulfonyl)carbamoyl]azetidin-1-yl}-5-cyano-2-(ethoxymethyl)nicotinate
ethyl 6-{4-[(benzylsulfonyl)carbamoyl]piperidin-1-yl}-5-cyano-2-(ethoxymethyl)nicotinate
ethyl 2-[(benzyloxy)methyl]-6-{3-[(benzylsulfonyl)carbamoyl]azetidin-1-yl}-5-cyanonicotinate
ethyl 2-[(benzyloxy)methyl]-6-{4-[(benzylsulfonyl)carbamoyl]piperidin-1-yl}-5-cyanonicotinate
ethyl 6-{3-[(benzylsulfonyl)carbamoyl]azetidin-1-yl}-5-cyano-2-(hydroxymethyl)nicotinate
ethyl 6-{3-[(benzylsulfonyl)carbamoyl]azetidin-1-yl}-5-cyano-2-ethoxynicotinate
ethyl 5-cyano-2-ethoxy-6-(3-{[(4-fluorobenzyl)sulfonyl]carbamoyl}azetidin-1-yl)nicotinate
ethyl 5-cyano-2-ethoxy-6-(3-{[(2-fluorobenzyl)sulfonyl]carbamoyl}azetidin-1-yl)nicotinate
ethyl 5-cyano-6-(3-{[(2,4-difluorobenzyl)sulfonyl]carbamoyl}azetidin-1-yl)-2-ethoxynicotinate
ethyl 6-{3-[(benzylsulfonyl)carbamoyl]azetidin-1-yl}-5-cyano-2-{[(3,4-dimethoxybenzyl)oxy]methyl}nicotinate
ethyl 5-chloro-6-(4-{[(4-chlorobenzyl)sulfonyl]carbamoyl}piperidin-1-yl)-2-(methylthio)nicotinate
ethyl 6-{4-[(benzylsulfonyl)carbamoyl]piperidin-1-yl}-5-fluoro-2-(methylthio)nicotinate
ethyl 6-{4-[(benzylsulfonyl)carbamoyl]piperidin-1-yl}-5-cyano-2-(2-methoxyethyl)nicotinate
ethyl 6-{4-[(benzylsulfonyl)carbamoyl]piperidin-1-yl}-2-chloro-5-fluoronicotinate
ethyl 6-{4-[(benzylsulfonyl)carbamoyl]piperidin-1-yl}-5-cyano-2-(1H-1,2,4-triazol-1-ylmethyl)nicotinate
ethyl 6-{4-[(benzylsulfonyl)carbamoyl]piperidin-1-yl}-5-cyano-2-(1H-1,2,3-triazol-1-ylmethyl)nicotinate
ethyl 6-{4-[(benzylsulfonyl)carbamoyl]piperidin-1-yl}-5-cyano-2-(1H-imidazol-1-ylmethyl)nicotinate
isopropyl 6-{4-[(benzylsulfonyl)carbamoyl]piperidin-1-yl}-2,5-dicyanonicotinate
1-(5-butyryl-3-cyano-6-methoxypyridin-2-yl)-N-[(4-fluorobenzyl)sulfonyl]piperidine-4-carboxamide
1-(5-butyryl-3-cyano-6-methoxypyridin-2-yl)-N-[(4-chlorobenzyl)sulfonyl]piperidine-4-carboxamide
N-(benzylsulfonyl)-1-(5-butyryl-3-cyano-6-methoxypyridin-2-yl)piperidine-4-carboxamide
ethyl 6-{4-[(benzylsulfonyl)carbamoyl]piperidin-1-yl}-5-chloro-2-(methylthio)nicotinate
isopropyl 6-(4-{[(4-chlorobenzyl)sulfonyl]carbamoyl}piperidin-1-yl)-5-cyano-2-methoxynicotinate
isopropyl 5-cyano-6-(4-{[(4-fluorobenzyl)sulfonyl]carbamoyl}piperidin-1-yl)-2-methoxynicotinate
ethyl 6-{3-[(benzylsulfonyl)carbamoyl]azetidin-1-yl}-5-cyano-2-(methylthio)nicotinate
ethyl 6-{4-[(benzylsulfonyl)carbamoyl]piperidin-1-yl}-5-cyano-2-(methylthio)nicotinate
ethyl 6-{4-[(benzylsulfonyl)carbamoyl]piperidin-1-yl}-2,5-dichloronicotinate
isopropyl 6-{4-[(benzylsulfonyl)carbamoyl]piperidin-1-yl}-5-cyano-2-methoxynicotinate
N-(benzylsulfonyl)-1-[3-cyano-6-(methylthio)-5-pentanoylpyridin-2-yl]piperidine-4-carboxamide
1-[3-cyano-6-(methylthio)-5-pentanoylpyridin-2-yl]-N-[(4-methoxybenzyl)sulfonyl]piperidine-4-carboxamide;
or a pharmaceutically acceptable salt thereof.

21. A pharmaceutical composition comprising a compound according to claim 1 in combination with a pharmaceutically acceptable adjuvant, diluent and/or carrier.

22-24. (canceled)

25. A method of treatment of a platelet aggregation disorder comprising administering to a patient suffering from such a disorder a therapeutically effective amount of a compound according to claim 1.
